SEQUENCE LISTING (94 sequences)

SEQ ID NO: 1 - DNA, 1086 bp, Arabidopsis thaliana
atggctttagaagaggaggaagagagtcaaaacgcaccgttttgtgttcttgatggtcttttctgtgaggaagagagtgagtttcacgaacaagtagatttgtgcgacgagagtgttgaaaagtttccttttttaaatctgggtttgtctgatcatgatatgttgtgggatgatgatgagttatcaactttgatttcgaaacaagaaccgtgtctttatgacgaaatcttagatgatgagtttctggttttgtgtcgtgaaaaggctcttgattggatttttaaagtgaaatctcattatgggtttaattcattgacggctcttttagctgttaattacttcgataggtttattacaagcaggaagtttcagacagataagccatggatgtctcagcttactgctttggcttgtctgtctttagctgctaaggttgaagagatccgtgttccttttctcttagattttcaagtggaagaagcaagatatgtctttgaagctaagactatacagagaatggagcttcttgttctgtctactcttgactggaggatgcatcctgtgactccaatctcgtttttcgatcacattattcgacgatacagctttaaatctcatcatcaattggagttcttgagtagatgtgaatctttattactctccattattcctgattcgagatttctgagttttagtccttctgtgttagccactgcaataatggtctctgttattagagatttgaagatgtgtgacgaagctgtataccaatctcagctcatgactctactcaaagttgattcggagaaggtaaataaatgctatgagttagtgttagaccacagtccaagcaagaaaaggatgatgaattggatgcaacaacccgctagtccgatcggtgtgtttgatgcgtcattcagttctgatagctcgaatgagtcgtgggttgtgtctgcttctgcttcagtgtcgtcttcaccatcttcagagcctttgctcaagaggagaagagtgcaagagcagcagatgaggctatcttcaataaaccgaatgtttttcgatgtgcttagtagtagtcctcgctaa

SEQ ID NO: 2 - protein, 361 aa, Arabidopsis thaliana
MALEEEEESQNAPFCVLDGLFCEEESEFHEQVDLCDESVEKFPFLNLGLSDHDMLWDDDELSTLISKQEPCLYDEILDDEFLVLCREKALDWIFKVKSHYGFNSLTALLAVNYFDRFITSRKFQTDKPWMSQLTALACLSLAAKVEEIRVPFLLDFQVEEARYVFEAKTIQRMELLVLSTLDWRMHPVTPISFFDHIIRRYSFKSHHQLEFLSRCESLLLSIIPDSRFLSFSPSVLATAIMVSVIRDLKMCDEAVYQSQLMTLLKVDSEKVNKCYELVLDHSPSKKRMMNWMQQPASPIGVFDASFSSDSSNESWVVSASASVSSSPSSEPLLKRRRVQEQQMRLSSINRMFFDVLSSSPR

SEQ ID NO: 3 - DNA, 654 bp, Oryza sativa
cttctacatcggcttaggtgtagcaacacgactttattattattattattattattattattattttacaaaaatataaaatagatcagtccctcaccacaagtagagcaagttggtgagttattgtaaagttctacaaagctaatttaaaagttattgcattaacttatttcatattacaaacaagagtgtcaatggaacaatgaaaaccatatgacatactataattttgtttttattattgaaattatataattcaaagagaataaatccacatagccgtaaagttctacatgtggtgcattaccaaaatatatatagcttacaaaacatgacaagcttagtttgaaaaattgcaatccttatcacattgacacataaagtgagtgatgagtcataatattattttctttgctacccatcatgtatatatgatagccacaaagttactttgatgatgatatcaaagaacatttttaggtgcacctaacagaatatccaaataatatgactcacttagatcataatagagcatcaagtaaaactaacactctaaagcaaccgatgggaaagcatctataaatagacaagcacaatgaaaatcctcatcatccttcaccacaattcaaatattatagttgaagcatagtagta

SEQ ID NO: 4 - DNA, 54 bp, Artificial sequence
  primer prm0360
ggggacaagtttgtacaaaaaagcaggcttcacaatggctttagaagaggagga

SEQ ID NO: 5 - DNA, 51 bp, Artificial sequence
  primer prm0361
ggggaccactttgtacaagaaagctgggtttagcgaggactactataagca

SEQ ID NO: 6 - DNA, 1140 bp, Antirrhinum majus
gagagaagagatggctcaaagctcaactctaggcctctctttcttcacacctttgtttgacttagaatgaaatgaatgcagcttcacacaactctcttctcctcagaagatgtatcaacaaaactctccatcactctgttttgatgctctgtactgtgaggaagaacaaaactgggacaatggtgaaatcatcaatgactgtttcattgaagaacaagaacccttttctgatttattgaaacatgatttgttatgtggtgtagatgatgatgatgatgataaagaagagcttagctctttattgtgtaaagagcaggaatatgaactgtacagagtccttgaggacaatccatctctagcaaaagctagagatgaggctgttgaatggatgtttaaggtcattgggtactattctttttctgctctcactgcggttcttgcagttaactatttggatagatttctatgcacatttcagtttcaacaagataagccatggatgtatcagttggctgctgtggcttgtctctctttggctgctaaagttgaagaaactcaagtccctcttctgttagaccttcaagttgaggaatctaagtatgtgtttgagtcaaaaaccattcaaagaatggagcttttggtgctttcaacacttaaatggaagatgaatccagtcaccccaatttcattccttgagtacattgctaggaggctagcattgaagagccatctttgtaaagagttcctcaacagatgtgaatgcctccttttgtcccttattaccgattgtagattcatgtgccatcttccatctgcattggccactgcaacgatgctgtatgttataagcagcttagagccctgcattggtgtggagtaccaagatcaactcatcaacattcttggaatcaacaaggacaaagtggaggaatgttgtaagttaatacaagaagtggccacaagtgttcattttcaatcaggcaacaaaagaaagtttggatctttgccttatagccccaaaggggtagtggacatctcattcagttgtgatgattcatggccgttggattcaactgcatcagtttcttcctcaccagagcatttgtccaagaaaatcaagacccaaaatccagaccatga

SEQ ID NO: 7 - protein, 343 aa, Antirrhinum majus
MYQQNSPSLCFDALYCEEEQNWDNGEIINDCFIEEQEPFSDLLKHDLLCGVDDDDDDKEELSSLLCKEQEYELYRVLEDNPSLAKARDEAVEWMFKVIGYYSFSALTAVLAVNYLDRFLCTFQFQQDKPWMYQLAAVACLSLAAKVEETQVPLLLDLQVEESKYVFESKTIQRMELLVLSTLKWKMNPVTPISFLEYIARRLALKSHLCKEFLNRCECLLLSLITDCRFMCHLPSALATATMLYVISSLEPCIGVEYQDQLINILGINKDKVEECCKLIQEVATSVHFQSGNKRKFGSLPYSPKGVVDISFSCDDSWPLDSTASVSSSPEHLSKKIKTQNPDH

SEQ ID NO: 8 - DNA, 1451 bp, Antirrhinum majus
taaaaaaaagcgtaaaattaaacagaaaaaaatatgatattaagcccaagattcaatcttttgagcttttcttaaagataatgagcagagccccctgtatcttcttctccgaagaagatgttgttttcacattctcaacaaacccatctccaaaatccaatctttgatgctcttctctgtaacgaggagcatttcgatgaagatttggatcttgggtccgggttaaaagacccgggttttataaatcagattcatcataatcaaaaaaaagaagaaccatttactacttttctgtttgagcacgaccttttgtgggaagatgacgagcttgttaatctcttgtctaaggagaaagaacaagaacaacaagcccatttggggtacgatgatgtaatggactctgatgggtttttgaaaagggtgagaaatgaagggattaagtggatgttgaaggtgattggacactatgggttcaatgcgatgactgctgttttagctgtgaattattatgatagatttattacaaacgttgggtttcaaaaggataagccttggatgagtcaattggctgctgttgcttgtctttctgtaaaagtggaggagactcaagtgcctctgttgctggattttcaagtagaggaatcaaagtatgtgtttgaggcaaagactatacagaggatggagcttttggtgctcactactttgaaatggaagatgaaccctgtgacgcctatctcgttctttgaccacattgtgaggaggtttgagttgatgaacaatgtgcaatgcgagtttatgaagaggtgtgagagtgtcattctctccatcatcaccgattatcgatttgtgcgctatcttccttctgttgttgctgctgcaaccatgatatatgtaatcaaagagctttatccttgtgatgcattggaataccagaatgagtttgtgactgtgctgagaactagcaaggaaaagactgatgattgccatatgctaatcactgaagtaatcaacaatcaaagctacatcctttgtcacaagcgcaagtacggttccataccaagcagtccaaatggtgtgatcgatgcctatttcagctctgatggctctaacgattcgtggtcagcagtgtcatccgtttcatcatcaccagagcccgtgtttaagagaatcagagccattgggggggctaatcctcctcattgaactcatttcttattttatctgatatttagcaacggtacttcataatcgctcttttgctatggttttttccgtcataagacgatgcagttatgttacatttctgttatatcttgctgtgatggatcagacatgttttaacagacaattgactcttatcacctctttgattgagggatggagagccaaagggattatgtgagtttttgttcctggagtaaaatcgatgaacttttagtattaatgaaaaa

SEQ ID NO: 9 - protein, 361 aa, Antirrhinum majus
MLFSHSQQTHLQNPIFDALLCNEEHFDEDLDLGSGLKDPGFINQIHHNQKKEEPFTTFLFEHDLLWEDDELVNLLSKEKEQEQQAHLGYDDVMDSDGFLKRVRNEGIKWMLKVIGHYGFNAMTAVLAVNYYDRFITNVGFQKDKPWMSQLAAVACLSVKVEETQVPLLLDFQVEESKYVFEAKTIQRMELLVLTTLKWKMNPVTPISFFDHIVRRFELMNNVQCEFMKRCESVILSIITDYRFVRYLPSVVAAATMIYVIKELYPCDALEYQNEFVTVLRTSKEKTDDCHMLITEVINNQSYILCHKRKYGSIPSSPNGVIDAYFSSDGSNDSWSAVSSVSSSPEPVFKRIRAIGGANPPH

SEQ ID NO: 10 - DNA, 1646 bp, Arabidopsis thaliana
atcactctccgaaacccacttccagctttttcctctctctttctctctctagtctctcttttgtagctctcccctgctaagctaaccactgcacgtttccatagagaggaaagatgagtctctctccgagagattttctctctatcatcttatcttcttccgtgtaatgctctgagccaaaacccaataactaaatcaacaacaatatagaagagaagagaaagatcttatctttcttctcattcttgagtttagtcccccacaatggcgattcggaaggaggaagaaagtagagaagaacagagcaattcgtttcttcttgatgctctctactgcgaagaagagaaatgggacgatgaaggagaagaagttgaagaaaactcttccttgtcttcttcttcttctccattcgttgttttgcaacaagatttgttctgggaagatgaagatctggttacactcttctccaaagaagaagaacaaggactcagctgtctcgatgatgtttatctttccacggatcgaaaagaagctgttggttggattctgagagtcaacgctcattatggcttctctactttagcagctgttttagccataacttatctcgataagttcatctgtagctacagcttacagagagacaaaccatggatgcttcagctcgtttctgtcgcgtgtctctcattagctgctaaagtcgaagaaacccaagtccctcttcttctagactttcaagtggaggagacaaagtatgtgtttgaagcaaaaaccatacagagaatggagctactgattctgtctactctcgagtggaagatgcatctcattactccaatttcgttcgtagaccacattatcaggagattgggacttaagaacaatgctcactgggatttcctcaacaaatgccaccgtctcctcctctctgtaatctccgattcaagatttgtcgggtacctcccatcagtagttgccgcagctaccatgatgcgaattatagagcaagttgatccctttgaccctctttcataccaaactaatctcctcggtgtccttaacttaaccaaggaaaaggtgaaaacttgctacgatctaatcctccaactaccagtggaccgcatcggtttacagatccaaatccaatcttccaagaaacgcaagagtcacgattcatcatcatcgttgaacagtccaagctgcgtgattgatgcaaaccctttcaatagcgacgaaagctcaaacgattcgtggtcagcgagttcgtgcaacccaccaacgtcgtcgtcgtccccgcagcaacaacctccattgaagaagatgagaggagctgaagagaatgagaagaagaagccgattttgcatctgccatgggcaatcgtagccactccataatcgaaagctcgatttcgtttatatgatatttactgtttttttaaactttgagaacaatctttgttgtattaagctttacccgtttgcatatacgaaatgtcgcgaatgcccttacgtgccatggcttgatagagttaatgggtaaagggtattcatgacatttgactgcatgggatgtgacgaaggagagaattagaaataataataataatattgcgtaaattttgaggcttgcccaatctttgggccgt

SEQ ID NO: 11 - protein, 376 aa, Arabidopsis thaliana
MAIRKEEESREEQSNSFLLDALYCEEEKWDDEGEEVEENSSLSSSSSPFVVLQQDLFWEDEDLVTLFSKEEEQGLSCLDDVYLSTDRKEAVGWILRVNAHYGFSTLAAVLAITYLDKFICSYSLQRDKPWMLQLVSVACLSLAAKVEETQVPLLLDFQVEETKYVFEAKTIQRMELLILSTLEWKMHLITPISFVDHIIRRLGLKNNAHWDFLNKCHRLLLSVISDSRFVGYLPSVVAAATMMRIIEQVDPFDPLSYQTNLLGVLNLTKEKVKTCYDLILQLPVDRIGLQIQIQSSKKRKSHDSSSSLNSPSCVIDANPFNSDESSNDSWSASSCNPPTSSSSPQQQPPLKKMRGAEENEKKKPILHLPWAIVATP

SEQ ID NO: 12 - DNA, 1415 bp, Arabidopsis thaliana
aaggcgtgagataataaaaccctttggctttctcatagagatttgtccggtctcttgttcctctttctcctttcttcactgtagaatccgtcgaccaaacaactagctccaatggcataatgagcattgtagtttgcaatttcttcttccgtgaagaagaagaagatggctttggagaaagaggaagaagcgtcacaaaacggtgcgttttgtgtccttgatgggctctattgcgaggaagaaaccgggtttgtggaggacgatcttgatgacgatggagatttagattttctcgagaaatctgatgagagtgttgtaaagtttcagtttttacctcttttggatatgttcttatgggatgacgatgagattctgagtttgatttcaaaggaaaacgaaacgaatccatgttttggggaacaaatcttagatggctttttggtttcttgtaggaaagaggctttagattgggttcttagggttaaatctcattatgggtttacttcattgacggctatacttgctgtgaactacttcgataggtttatgacaagtataaagcttcagactgataagccatggatgtctcagcttgttgctgtggcttctttgtctttagctgctaaagttgaagagattcaagttccattgctcttagacctccaagtggaagaagcaagatatctctttgaagctaagacgattcaaagaatggagcttttgattctttctactcttcaatggagaatgcaccctgtgactccaatctctttctttgatcacattatccggcgatttggctctaaatggcaccagcaattagacttctgtaggaagtgtgagcgtcttctgatctctgttattgctgatacgaggtttatgaggtacttcccttctgtcttagctactgcaataatgatccttgtcttcgaggaattgaagccatgtgatgaagttgaataccaatctcaaataacgactctactcaaagtcaatcaggagaaagtaaatgaatgctatgaactgttgttggagcacaatccaagcaagaagaggatgatgaatttggttgatcaggacagtccaagtggtgtattagactttgatgacagctcaaatagctcctggaatgtctccactactgcttcagtgtcctcatcatcttcgtctccagagcctctgctcaagagaagaagagttcaggagcagcaaatgagattgccctcaataaaccgtatgtttctcgatgtgcttagtagtcctcgctagtacctttctttgatcaaatgtgtcaaaacataaattcaatctctcttttgcttattattatcggccatcggctacaatttgaaggcagaacattttgtgataactctaagttaattctgcctcttaaatcataatattcattgatc

SEQ ID NO: 13 - protein, 367 aa, Arabidopsis thaliana
MALEKEEEASQNGAFCVLDGLYCEEETGFVEDDLDDDGDLDFLEKSDESVVKFQFLPLLDMFLWDDDEILSLISKENETNPCFGEQILDGFLVSCRKEALDWVLRVKSHYGFTSLTAILAVNYFDRFMTSIKLQTDKPWMSQLVAVASLSLAAKVEEIQVPLLLDLQVEEARYLFEAKTIQRMELLILSTLQWRMHPVTPISFFDHIIRRFGSKWHQQLDFCRKCERLLISVIADTRFMRYFPSVLATAIMILVFEELKPCDEVEYQSQITTLLKVNQEKVNECYELLLEHNPSKKRMMNLVDQDSPSGVLDFDDSSNSSWNVSTTASVSSSSSSPEPLLKRRRVQEQQMRLPSINRMFLDVLSSPR

SEQ ID NO: 14 - DNA, 1425 bp, Euphorbia esula
  misc_feature (1347)..(1347): n is a, c, g, or t
tttttttttcttctctgcctctctatccattccttctcttctctctgtctctgctatcaatagacccctagtgagagaaacagagataaagcaaattgtatggtgatatgatttgcaatgagattgagattgggattttattttctttgatggtataatgagcaaaggattgaatctcttcttctctgaagaagaacaaaaatggaagacagcactcagatttcattgatttttgatgggttgtactgcgaagaacaaggcattggtgaagattttgacgatgggaatgaagattatgtgaaaaaggagttatctttatcttctgttttgcttgagcaggacttgttttggaccgatgatgagttgttaaatctgatttcaaaagaaaaagagactcattttagttttggggatttttcctctcatgggtctttaatggtggctcgtaaagaggcaatagattggattttgagggtaaaagggttttatggattcaatgctttgagctgtgttcttgctgttaattattttgatagattcatttcgagtttagtttttacaagagataaaccatggatgggtcaacttgctgctgttgcttgtttatctttggctgctaaaatggaggagactcaagttcctcttcttctagatttacaagtggaagaatcaaagtatgtgtttgaggcaaagactataaagagaatggagcttcttgtgctctctactcttcaatggaggatgaatcctgtgaccccaatttgctactttgatcacattataaggaggctaggacttaaaaaccatctgcattgggaatttttgaggagatgtgagcttttacttctctctgtcatttctgattcaagattcatgagttatgcaccttctatattagcaacttcaattatgatccatgtgattaaggaggttgacccatttagtcaaatggaataccagaaccagcttttggatgtgatcaaaatcaacaaggaggaagtgaaccagtgttacaagctcatcttggagctatcgggtaagcaagatcaagggtacaaacgcaagtatccctcaagacccgggagcccaaatggtgtgattgatgcctattttagtggagatagctcgaatgattcgtggggagtttcttcctcaatctcatcatcaccatcgattcctcgatttaaaaggatcaaatcccaggatcaacagatgaggttgccttcaataaaccgtatgtttgtggatgtgcttagtagtcctcattgatcttttacttttgttatctattgcctacttcgaaacaatgccattataaatttctctattttgcatattatttgtatncccgtggttacttgggtacatttccggcctaatatttgaactcactcaagctagacaaattgaaaaaaaaaaaaaaaa

SEQ ID NO: 15 - protein, 355 aa, Euphorbia esula
MEDSTQISLIFDGLYCEEQGIGEDFDDGNEDYVKKELSLSSVLLEQDLFWTDDELLNLISKEKETHFSFGDFSSHGSLMVARKEAIDWILRVKGFYGFNALSCVLAVNYFDRFISSLVFTRDKPWMGQLAAVACLSLAAKMEETQVPLLLDLQVEESKYVFEAKTIKRMELLVLSTLQWRMNPVTPICYFDHIIRRLGLKNHLHWEFLRRCELLLLSVISDSRFMSYAPSILATSIMIHVIKEVDPFSQMEYQNQLLDVIKINKEEVNQCYKLILELSGKQDQGYKRKYPSRPGSPNGVIDAYFSGDSSNDSWGVSSSISSSPSIPRFKRIKSQDQQMRLPSINRMFVDVLSSPH

SEQ ID NO: 16 - DNA, 1501 bp, Euphorbia esula
aaagaattcctacaacctatgccttcttccccactcagatacccaaataaaaacacatacccttagataactggtcactacccctcttcctgctctttttgtttctttgacaaagagagagaaatggctcaaactgagtctttaactctctctttaatgttcatcttctattcctttgccctataatgaaccccaatctcaattktatcttcttagaaagaagatggcaaatcattctccattatttctctatgatgctctttactgctcagaagaagataactgggaaggagaagttgttgatatttttcatgaacaagaagatcaaggagaaaacacctctgtctttccccaaaattcttccccagtagacttaaattgggaagaagatgagcttacgtctgtattttctaaacaagagcaaaaccaactatataaaaaactagaaatcaacccatgtctagctaaatctcgccgtgatgctgtggattggatgatgaaagtcaatgcccattactctttcactgctttgacttcagttttggccgttaattttcttgatagattcctttttagcttcgatcttcaaacggagaagccatggatgacccagctcacagctgtagcttgtctctccttagcagcaaaagtagaagagacacaagtcccacttttattggaccttcaggtggtggacagtaagtatgtgtttgaggctaaaactatacaaagaatggagcttttggtgctttctactcttcaatggagaatgaaccctgtaactccattatcatttattgattacatgacaagaaggcttggttttaaggattatctttgctgggaatttatccggagatgcgagcttattgttctctctataatctcagatatgagatttataccttatctccctagtgaaattgcttccgcaataatgctacatgtgattaatggtatagaacccagtcttggagatgaattcgaaacccagctattcgggattcttggaattgataaggagaaggtgaataattgcagagaaatgataatcgagttaggatcaagatattacggcaaccaatcaaacaaaagaaaatacgggtcggatccgggtagtccaaattgcgtaatggatgtctcatttagttcagataattcaaacgattcttgggcagtcggatctaaatcatcatcagtgtcttcctcacccgcggcgaagaaactcagggcagtttcagggatgaaccatgaaaatgcgataatactttcctaagcatgaacattttttaatccttaaaatctttttaatttatttcatttgaatccccttttgaggacttggttgtattaaactgttaataattgatgttgttagttaaattgccgggcatctctgcttctccaatctcaattaaaatcttaattagaattttggaagcagagatggttggcattttatccggaaattagtagtaaaagagaaattgcactaaaaaaa

SEQ ID NO: 17 - protein, 350 aa, Euphorbia esula
MANHSPLFLYDALYCSEEDNWEGEVVDIFHEQEDQGENTSVFPQNSSPVDLNWEEDELTSVFSKQEQNQLYKKLEINPCLAKSRRDAVDWMMKVNAHYSFTALTSVLAVNFLDRFLFSFDLQTEKPWMTQLTAVACLSLAAKVEETQVPLLLDLQVVDSKYVFEAKTIQRMELLVLSTLQWRMNPVTPLSFIDYMTRRLGFKDYLCWEFIRRCELIVLSIISDMRFIPYLPSEIASAIMLHVINGIEPSLGDEFETQLFGILGIDKEKVNNCREMIIELGSRYYGNQSNKRKYGSDPGSPNCVMDVSFSSDNSNDSWAVGSKSSSVSSSPAAKKLRAVSGMNHENAIILS

SEQ ID NO: 18 - DNA, 1193 bp, Helianthus annuus
aatacccaatggccattttatcacgatattcatcttcgaacacactcttctgcattgaagaacaagttcatgaagatgaagatgagttaacacatcaagattcctctgcaatccatccattagacctacaagatttgtgttgggaacatgaagaacttgtctctcttttcacaaaagaagaagaacaacaaaagcaaaccccttggccttcttcttgtactttgtcttttcgtaaagaggctgtggattggatccttaaagtcaaaggttgtcatggattcacacctctaacagccattttagccatcaattatcttgatcggtttctgtccagtctccattttcagaaagctaacacaccttggatgattcaccttgttgctgttacttgtctttctttggctgctaaaattcaagaaactcatgtgcctttgctcttagatcttcagctagaggagagtaagttcttgtttgaggccaagaacatacaaaagacggagcttttggtgatgtcaacactgaaatggaggatgaacctagtgacaccaatctcatttcttgatcacattgtaagaaggcttggattatcaaatcatcttcattgggatttcttcaagaaatgtgaagctatgattctttacctagtggctgattcaagatttgtgtgctataaaccatctgtgttggcaaccgctacaatgctttgtgttgtagaggaaatcgacccgaccaattccattggctacaaaagtcaacttctggatcttctcaaaaccactaaggaccacataaatgagtgttacaagcttgttatggatctatcctatgataatcacaacaaaggaaagcgtgatgaaaacgagagaacaatttatccggttagtccagctggttttattggttttatgtgccacgaaagttcaaatgattcatgatcctcgctcaagaattgatcaataattagggtttggctcacttgtaagctttaaaccactcgcaagcactcgttatccataacctatacaatcagcagaagttgcgctaataatagacccgtcggtccaccactagttatgttgtatggtatggtctttaatttctctgttgttttaggtcgtttttaatgtgagataagttaaactcggtgatgttatcatgtcttattcaagcaatgaatttatatattttacatc

SEQ ID NO: 19 - protein, 308 aa, Helianthus annuus
MAILSRYSSSNTLFCIEEQVHEDEDELTHQDSSAIHPLDLQDLCWEHEELVSLFTKEEEQQKQTPWPSSCTLSFRKEAVDWILKVKGCHGFTPLTAILAINYLDRFLSSLHFQKANTPWMIHLVAVTCLSLAAKIQETHVPLLLDLQLEESKFLFEAKNIQKTELLVMSTLKWRMNLVTPISFLDHIVRRLGLSNHLHWDFFKKCEAMILYLVADSRFVCYKPSVLATATMLCVVEEIDPTNSIGYKSQLLDLLKTTKDHINECYKLVMDLSYDNHNKGKRDENERTIYPVSPAGFIGFMCHESSNDS

SEQ ID NO: 20 - DNA, 1400 bp, Helianthus tuberosus
ttgaaccttcatttcttttcttttcttctttctaatcaccaaccccaatggccattttatcaccatattcatcttctttcttagacacactcttttgcaatgaacaacaagatcatgaatatcatgaatatgagtatgaagatgaatttacacaaaccaccctcacagattcatctgatctccatcttccccccctggaccaactagatttgtcatgggaacatgaagagcttgtgtccttgttcacaaaagaacaagagcagcaaaaacaaaccccttgtactctctcttttggcaaaactagtccctcagtttttgctgctcgtaaagaggctgtagattggatccttaaggtcaaaagttgttatggattcacacctcttacagccattttagccatcaattatcttgataggtttctttctagcctccattttcaagaagataaaccttggatgattcaacttgttgctgttagttgtctctctttagctgctaaagttgaagaaactcaagtgccactcttactagatcttcaagtagaggacactaagtacttgtttgaggctaaaaacatacaaaaaatggagcttttggtgatgtcaactttgaaatggaggatgaacccagtgacaccaatctcatttcttgatcacattgtaagaaggcttggattaactgatcatgttcattgggattttttcaagaaatgtgaagctatgatcctttgtttagtttcagattcaagattcgtgtgttataaaccatccgtgttggccacagctacaatgcttcacgttgtagatgaaattgatcctcccaattgtattgactacaaaagtcaacttctggatcttctcaaaaccactaaggacgacataaacgagtgttacgagctcattgtcgagctagcttacgatcatcacaacaaacgaaaacatgatgcaaacgagacaacaaccaatccggttagtccagctggcgtgatcgatttcacttgtgatgaaagttcaaatgagtcatgggaacttaatgctcatcatttccgcgagccttcattcaagaaaacaagaatggattcaacaattcgggttcgggtttggttcacttataagctttaatcgagggtagttgtaaacatgtaatccgcatgcacgctattaatcctacggtccactactacatataatcggcctataaaattataggttaagatgaccagtcgtaggcgtcgagatgtccttatggttggtcaatttctctatggttttaggtcgtttttaatgtgagataaattaaattcggtatgttaagtctttatcaagcaatggacgttatatttattgtttgatattgagaattaaattccatgggaaaaaaaaaaaaaaaaaaaaaaaaaa

SEQ ID NO: 21 - protein, 357 aa, Helianthus tuberosus
MAILSPYSSSFLDTLFCNEQQDHEYHEYEYEDEFTQTTLTDSSDLHLPPLDQLDLSWEHEELVSLFTKEQEQQKQTPCTLSFGKTSPSVFAARKEAVDWILKVKSCYGFTPLTAILAINYLDRFLSSLHFQEDKPWMIQLVAVSCLSLAAKVEETQVPLLLDLQVEDTKYLFEAKNIQKMELLVMSTLKWRMNPVTPISFLDHIVRRLGLTDHVHWDFFKKCEAMILCLVSDSRFVCYKPSVLATATMLHVVDEIDPPNCIDYKSQLLDLLKTTKDDINECYELIVELAYDHHNKRKHDANETTTNPVSPAGVIDFTCDESSNESWELNAHHFREPSFKKTRMDSTIRVRVWFTYKL

SEQ ID NO: 22 - DNA, 1630 bp, Lagenaria siceraria
aagagcacaagaaagctcatccttaactgctttcttcacagggctggttgcttctgtcgacggggattcgggtgtgagctttttatttttctccgtgtctcgtaccccctcggagggttagtcctatgagctcccggtgtcctcgctgctcaattcagctttgaggtcctctccttcttttccactctgaataagatagaggaaaagagagaaaccccagcttacagccatgggtgaggattggtcttcttcgccattttcctctgtttttctccatttccttctctcaagctctacaatgaaccaaaatcacagcctctttcttctcagaagaagatggtaccaccgtatgcgcttgattctctttattgttcagaagaccactgggagaacgacgacgaagaagaagaaaggggttttcatgagcaaccttattctaatttaacaaccgaatcaagttctccgattctggcagtggcagagcaggatctgttttgggaaaacgatgaattaatttctctgttctcaagagagaagcctaatgaactgtttaaaaccattcagattgacccttctcttgctgccgcccgacgaagcgccgtcgggtggatgctgaaggttaatgcccattattctttctctgctctcactgcggttctcgccgtcgattatttggatcggtttctgtcctgttttcattttcaaagagacaagccatggatgtctcagcttgctgctgttgcttgtatctctcttgctgccaaagtagaggagacccaagtccctcttttattggacctacaagtggaagacagtagatatctatttgaagccaagacaattaagaaaatggagcttcttgtgctctctacgcttcaatggcggatgaatcctgttaccccattttcttttgtggattatatctcaaggaggcttggattcaaggaacatatctgctgggaaattctttggcagtgtgagcgaactattctctctgttattttagagtcagattttatgtcctttcttccttctgtaatggccaccgctacaatgctgcacgttttcaaagctatggaagaacccaccctcagcgttgaatacgattcccagcttcttaacatcctcggaatcgacaaggggaatgtggaagaatgctgtaagctgatctcaaatgcatcaagaagaaacggcaaccaattcaagaaacgtaaaattgggtcgattccgggtagcccgaacggcgtgatggacgtgtcattcagctccgatagctcgaacgactcgtggtcagtggcctcgtcagtttcatcttcgccagagccattaacgaagaagaacagagccaatggatcaatgtctggagattgcgaaacattcagaaccctctcttaattaacttccctttcttctttttcctcgtaatccttgtatgttgaataagaattagaatcaatctttttatttatcgaattctgcgagttaaattgccttaccatctctgcagttaagagccaatggatgggcaatcggaagtttaaaacgcagagatggctggcattttatccggatgcaaaggacattgaacgtataacaatgaagagtagtttaagttgtttaaaaagaaaaa

SEQ ID NO: 23 - protein, 352 aa, Lagenaria siceraria
MVPPYALDSLYCSEDHWENDDEEEERGFHEQPYSNLTTESSSPILAVAEQDLFWENDELISLFSREKPNELFKTIQIDPSLAAARRSAVGWMLKVNAHYSFSALTAVLAVDYLDRFLSCFHFQRDKPWMSQLAAVACISLAAKVEETQVPLLLDLQVEDSRYLFEAKTIKKMELLVLSTLQWRMNPVTPFSFVDYISRRLGFKEHICWEILWQCERTILSVILESDFMSFLPSVMATATMLHVFKAMEEPTLSVEYDSQLLNILGIDKGNVEECCKLISNASRRNGNQFKKRKIGSIPGSPNGVMDVSFSSDSSNDSWSVASSVSSSPEPLTKKNRANGSMSGDCETFRTLS

SEQ ID NO: 24 - DNA, 1565 bp, Lagenaria siceraria
tggacacgaatttcttcgactagtaagcaccaaaagctccataaaagagaaaaaaaaaaggggggaaatggggtttctcttcctctccctataatgactccgcaatactcaatcctcttcttccaagaagaagaagatgaagaagatggctttgcactcaaataaacacagaacccaacgcctccataactctctcttcttcttcgacttcctccactgcactgaacaacaacaccttcaaacagagcatcccattttccttaacaatgggggcaccaacgacttccctcttttccaacaaacaaccacccatttccttgtttacgaagacgaggagctcaatcatttgttgtccaaagaaaaggaccaaaatctccaaaccggtgctgttttgaaaaccttggttcaaacagataatgctctgtctctcgctagaacagaggccatcgactggttgcttaaagttaatgccttttatggtttctcctctctcacagctctcttagccattaattacctcgatagaatcctctctgggccctattttcaaagagataagccatggatgcttcagcttgctgctgtaacttgcatctctttagctgctaaagtcgaagaaattcgtgtccctcttcttctagacctccaggtggaagattcaaagtacatttttgaagcgaaaacgatacagaggatggagcttttagtgcttactgctctgcaatggaagatgcacccagtggcccctgtttcgtttcttggcattatcacaaaaggacttggaatgaagaatcagtacattcaaagagagtttcttagacgctgtgagcgtattcttctctctctcgtctctgattcgagatcggtggggattcttccttctataatggcggtatcagcaatggtgagcgttgttgaagagatgggaaactgtaacccattggaggagtttcaggatcagcttcttaatgccctcaaaataaataaggggagagtgaaggagtgttgtaaagtgataatggaggcaaaaataaaaggatcagggaagaggaagcatgtggaggaggaagcagaagcagaagcagaatcagaatcatcagaagcagaaacagagggagaagcagaagcagaagcagggagcccaaatggagtaatggaggctaatttcagctgtgaaagctccaacgattcgtgggaaatggggacgattgtgtcagaatacacacatttttcttcttcttcttcttcttcttccaaaagaatcagacccactcgatgaattatttgaataatgaattgaaccaaatttgcagagattcaatatccattacattagccttcccctgttctgaaatggcacattggcaaacacacaaaaaccagagacaaaaaagtggggaaaaacagagaagtaatgatgacgatgatgatgagagaggttgttcttctctttctttatagctcattaattatgttgtatatgatgaaattgatgagtatttgcttctattaatattgctctcttcttcatttcaaaaaaaaaaaaaagttgaccacgcgtggcc

SEQ ID NO: 25 - protein, 380 aa, Lagenaria siceraria
MKKMALHSNKHRTQRLHNSLFFFDFLHCTEQQHLQTEHPIFLNNGGTNDFPLFQQTTTHFLVYEDEELNHLLSKEKDQNLQTGAVLKTLVQTDNALSLARTEAIDWLLKVNAFYGFSSLTALLAINYLDRILSGPYFQRDKPWMLQLAAVTCISLAAKVEEIRVPLLLDLQVEDSKYIFEAKTIQRMELLVLTALQWKMHPVAPVSFLGIITKGLGMKNQYIQREFLRRCERILLSLVSDSRSVGILPSIMAVSAMVSVVEEMGNCNPLEEFQDQLLNALKINKGRVKECCKVIMEAKIKGSGKRKHVEEEAEAEAESESSEAETEGEAEAEAGSPNGVMEANFSCESSNDSWEMGTIVSEYTHFSSSSSSSSKRIRPTR

SEQ ID NO: 26 - DNA, 1518 bp, Lycopersicon esculentum
ctccttttggctcttcctattccctctctctcttccccttttttctgtcctttagagagagaaaaaaaatccataagccaagcagcagatatgttactgggtccaagattgagttttggcttaccttaaagataatgagtagagccttcattgtcttcttccctctagaagaaggagaagatggttttccctttagattcccagctccaaaatcctatttctgctcttcttgatggcctttactgtgaggaagatcgattcttggatgatgatttaggggaatggtctagtttagatgtcggaaatgaaaatgttaaaaagactctgcctttattagaatgtgacatgttttgggaacatgatgagcttgccacacttttatctaaggaaaatgagtttcatttgggttttcaatctttaatctcagatgggtctttaatgggggctagaaaagaggctttggattggatgttgagggtcattgcttactatggttttactgctaccactgctgttttagctgtgaactattttgataggtttgtgtctggatggtgctttcagaaagataagccttggatgagtcagcttgctgctgttgcctgtctttccattgctgctaaagtggaggagacccaagttccccttttgttagacctacaagttgctgattctagatttgtgtttgaggcaaagactatacagagaatggaactcttggtgctttctactcttaagtggaaaatgaatctggtgacaccattatctttcattgatcatattatgaggagatttggattcatgagcaacctgcatatggattttcttaagaagtgtgaacgcctcattcttgatatcatcactgattctaggctcttgcattatcctccatctgttattgcaactgcatcgatgttttatgtgatcaatgacattgagcctagcaatgctatggaataccaaaatcagctcatgagtgttcttaaagtcagaaaggacatctttgaggaatgccatgatcttattcttgagctaatggacactgcctgttacaagctctgccaaagcctcaagcgcaaacatcattcagtacctggtagtccaagtggtgttattgatgcatattttagtagtgagagctcgaatgaatcatggtcagtagcatcttcgatttcatcctcacctgagcctcagtataagagaaacaaaactcaagatcagcgaatgacactagctccactgggtagtaatcttcactgatcgatatcttgttctctagattacctagtatttcggcaatggtttactctcttttttggtatgttctcttaaaaatgcaattgcacaatgctctgatgctccatttaagttttactggacttaatttgtccgatgatcgtctagactatgtgaacatcaactccacccccctccttcattggacatgggattggtggagttttcccttgagttgaatcaatgctgctgaattatgttgaaaaaaaaaaaaaaaaaaaa

SEQ ID NO: 27 - protein, 359 aa, Lycopersicon esculentum
MVFPLDSQLQNPISALLDGLYCEEDRFLDDDLGEWSSLDVGNENVKKTLPLLECDMFWEHDELATLLSKENEFHLGFQSLISDGSLMGARKEALDWMLRVIAYYGFTATTAVLAVNYFDRFVSGWCFQKDKPWMSQLAAVACLSIAAKVEETQVPLLLDLQVADSRFVFEAKTIQRMELLVLSTLKWKMNLVTPLSFIDHIMRRFGFMSNLHMDFLKKCERLILDIITDSRLLHYPPSVIATASMFYVINDIEPSNAMEYQNQLMSVLKVRKDIFEECHDLILELMDTACYKLCQSLKRKHHSVPGSPSGVIDAYFSSESSNESWSVASSISSSPEPQYKRNKTQDQRMTLAPLGSNLH

SEQ ID NO: 28 - DNA, 1459 bp, Lycopersicon esculentum
acagagatattagaaggggaaaaaaatggcaatagagaataatgatcaatctttttttttagatgtgctttactgtgaagaagaagaagaaaaatggggtgatttgttagaggatgaagaaggggttattattaacccattgttgttatcttccgaaggaacaacaaaaactaattctttattattattacctctgcttctgttggaacaagatttgttttgggaagatgaagagcttctttcacttttcgttaaagaaaaagaaactcgttgttgttttgaaagttttgggagtgacccttttctctgttcagctcgtgttgatgttgttgaatggattcttaaagtgaatgctcattatgatttctcagcattgactgccattttagccattaattatcttgacaggtttctttctagccttcaatttcagaaagataagccatggatgactcaacttgctgctgtcacttgtctttctttagcggctaaagttgaagaaactcaagttccccttcttcttgacttccaagtggaggatgcaaaatatgtgtttgaggcaaagactatacaaagaatggagcttctggtactgtcatcactgaaatggaggatgaatccagtgaccccactttcatttcttgatcatattataagaaggcttgggctaaagaacaatgttcattgggaatttctcagaagatgtgaaagtcttcttctatctgtcatgattgattgtagatttgtacgttatatgccttctgtattggctactgcaattatgcttcatgttattcatcaaattgagccttgtaatgctattgactatcaaaatcaacttcttggggttctcaaaattagcaaggagaatgtgaataattgctatgaactcatatccgaagtgtcatcaaagcctattacatcacacaaacgcaaatatgatgaaaatcccagtagtccaagtggtgtaatagatccaatttacacttcagaaagttcaaatgattcatgggatttagatttgccttcgttcaagaaaagcaaagttcaagaacagcaaatgaaaatgtcatcatcattgagcagagtttttgtggaagctgttggtagtcctcattaaaatgtctcttttaaatattttaattacatattcaagaagtattttgctgttatgtgttagctgtggttgttggcagagaagagaagatgagtggctttattttttgcaggagtgtagtctactactactactgtgaagccagagagagaaagagaaaagacagaatatgtgcaatctttgtttttctctctatttatttcaattttctctctcaagtcactttcatgcatgcatacttttgatggactactctatttatattgcctttacttattagtacttaatatatatatatatatatatatatatatataaatcatataaaaaaaaaaaaaaaaa

SEQ ID NO: 29 - protein, 364 aa, Lycopersicon esculentum
MAIENNDQSFFLDVLYCEEEEEKWGDLLEDEEGVIINPLLLSSEGTTKTNSLLLLPLLLLEQDLFWEDEELLSLFVKEKETRCCFESFGSDPFLCSARVDVVEWILKVNAHYDFSALTAILAINYLDRFLSSLQFQKDKPWMTQLAAVTCLSLAAKVEETQVPLLLDFQVEDAKYVFEAKTIQRMELLVLSSLKWRMNPVTPLSFLDHIIRRLGLKNNVHWEFLRRCESLLLSVMIDCRFVRYMPSVLATAIMLHVIHQIEPCNAIDYQNQLLGVLKISKENVNNCYELISEVSSKPITSHKRKYDENPSSPSGVIDPIYTSESSNDSWDLDLPSFKKSKVQEQQMKMSSSLSRVFVEAVGSPH

SEQ ID NO: 30 - DNA, 1449 bp, Lycopersicon esculentum
acaaaccttatttttctttttctttttttaaattcagtttcttgaattagtatgaagtaaatgaaaacacaactaaacttcttcttctcagaagaagataatgtctcaccattatcaagaacaagaacaactagaagcacaaaagattccatttttattagattcactttactgtgaagaaaataatatactcactgaagtatcaatagagacagagagtttttcttcacatgatttgttatgggaagaagaagaacttacctctctgttttctaaagaaacagagtatgaaataagctacaatgtgttagaaaaaaaccagtcttttatttcatcaagaagagaatcagttgaatggatactcaaaacaactgcttattactctttttctgctcaaactggatttcttgcagttaattactttgatagatttctgttatttagttttaatcagtctctgaatcataagccatggatgaatcaacttgttgctgttacttgtctttcattagctgctaaagttgaagaaactgatgttcctctgcttcttgaccttcaagttgaggaatcaggatttttgtttgaatctaaaacaatacagagaatggagatgttgattctgtctacacttaaatggaagatgaatccagtaaccccattttcatttcttgattttataactagaagacttggattgaagcactgtctatctttggaatttctgaggagatgtgagaaagtgcttctttacacaattactgatgatagattcattggttaccttccttctgcaatggcatctgccacaatgttgcatgttcttgataggcttaagccttgcattggagaaaagtaccaagatcaacttttgggcattcttggaattgtcaaggagaaggtggaaggatgttacaggctaatacaagaagtggcttgcaacattgactttggttcaaataagagaaagtttgggacattaccagggagtccaacaggggttatggatatgtcatttagctcagattactccaatgactcatggtcagtggctacatcagttacttcatcacctgagccattgtccaagaagattagggagtcaaatgaatgactaattaaatgtcttttaaatatttccttattagtagactagttattattgttattattagatatgtataatgtccaataacagtgatccacttgtctcttagtttaagtattagtaattaattaagttcttgtttattcacatgttaattttgcttacatgtaaaaattcaaacccccttttgacaattatcacagctagctagctaatggaccatttgtttaatgctgtactgtctatagtggggctatgagattttgtactagttaaaagatggtttggcactttattccaaggaaataaaaacttgcatgaaaaacaaaaaaaaaaaaaaaaaa

SEQ ID NO: 31 - protein, 336 aa, Lycopersicon esculentum
MSHHYQEQEQLEAQKIPFLLDSLYCEENNILTEVSIETESFSSHDLLWEEEELTSLFSKETEYEISYNVLEKNQSFISSRRESVEWILKTTAYYSFSAQTGFLAVNYFDRFLLFSFNQSLNHKPWMNQLVAVTCLSLAAKVEETDVPLLLDLQVEESGFLFESKTIQRMEMLILSTLKWKMNPVTPFSFLDFITRRLGLKHCLSLEFLRRCEKVLLYTITDDRFIGYLPSAMASATMLHVLDRLKPCIGEKYQDQLLGILGIVKEKVEGCYRLIQEVACNIDFGSNKRKFGTLPGSPTGVMDMSFSSDYSNDSWSVATSVTSSPEPLSKKIRESNE

SEQ ID NO: 32 - DNA, 1861 bp, Medicago sativa
gaattcggcacgagctcttctgctacgactacctctccctatactctctactctttctagttctactacttttctttctttctctgttctctctctcttcttcatttcttcacattttcacacacacagagaagacagaacaaagaggaaaagagagagcgatggatgtgagactcttcagtactgtttccttctttttataatgaacaaaggaccacacaccctcttcttcactgaagaagatggctatccatcatcatcatcacaatcaccaacaacttcaacaacacacttcttctctttttgatgcactttactgtgatgaagaagaaaaatgggaagatgatgatgaaggagaagttgtagatgaaggagcacaaagtgatgtcacaacaacaaactatgatatattggactctacttcccttttacctctgcttttgttagaacagaacttgttcaatgaagatgaagaactcaacactcttttctccaaagagataactcaacaagaaacatattacgaggatctgaaaaatgtgatcaactttgactcactctctcaaccacgtcgtgaagctgttgaatggatgcttaaagtcaatgctcattatggtttctctgctctcactgcaacacttgctgttaactatcttgataggtttcttttaagcttccatttccaaaaagagaaaccatggatgattcagcttgttgctgttacttgcatctctttagctgctaaagttgaagaaactcaagttcctcttctcttagaccttcaagtgcaagatactaaatatgtgtttgaggcaaagactattcagagaatggagctattgattctgtcaacactgaaatggaagatgcatccagtgacaacacactcttttctagatcacattataagaaggcttggattgaaaactaatcttcattgggagtttcttaggcgctgtgagaatcttcttctatctgtacttttagattcaagatttgttggttgtgttccttctgtgttggccactgctacaatgttgcatgttatagaccagattgaacagagtgatgataatggtgtggattacaaaaatcagcttcttaatgttctcaaaatcagcaaggagaaagttgatgaatgttataatgcgattcttcatcttacaaatgcaaataattatggtcataaacgaaaatatgaagaaatccctggtagtccaagtggcgtaattgatgctgtttttagttctgatggttctaacgattcgtggacagtgggagcatcatcatattcaacctcagagcctgtgtttaagaagaccaagaatcaaggacaaaatatgaatttgtcaccgattaacagggtcattgtcggaattcttgccactgcaacctctccttaaaaccctctatccgttttctgtcctttttatttaaaaaaaaataaccatataaaaaattacccccaaaaaaaagatctatatttatttactatggttatgttcatgttgctactaaactctagttagtttagtagtctttctttctatctcttcatttccaacaatgtcccaaattcatttacatgaatctcttgaagaggcagtggcaagatgatgatagaggattaaaggaatggttaatttctgatgagttaaaaaggaaaggacaaagttggcaatgaagatttttattactatgagcagaaaagaaccctatgatatctgtttcatttcaaggcactgtttttttattttattcaatggttctcttctagaccatacccaatttggacatatttatatcatatttctataataaattgggaataatttttggtccaaaaaaaaaaaaaaaaaaa

SEQ ID NO: 33 - protein, 386 aa, Medicago sativa
MAIHHHHHNHQQLQQHTSSLFDALYCDEEEKWEDDDEGEVVDEGAQSDVTTTNYDILDSTSLLPLLLLEQNLFNEDEELNTLFSKEITQQETYYEDLKNVINFDSLSQPRREAVEWMLKVNAHYGFSALTATLAVNYLDRFLLSFHFQKEKPWMIQLVAVTCISLAAKVEETQVPLLLDLQVQDTKYVFEAKTIQRMELLILSTLKWKMHPVTTHSFLDHIIRRLGLKTNLHWEFLRRCENLLLSVLLDSRFVGCVPSVLATATMLHVIDQIEQSDDNGVDYKNQLLNVLKISKEKVDECYNAILHLTNANNYGHKRKYEEIPGSPSGVIDAVFSSDGSNDSWTVGASSYSTSEPVFKKTKNQGQNMNLSPINRVIVGILATATSP

SEQ ID NO: 34 - DNA, 1679 bp, Nicotiana tabacum
aaacgagtctctgtgtactcctcctcctatagcttttctctcttcttctcttcacacctcccacaacacacaatcagacaaaatagagaggaaaatgagtatggtgaaaaagctttgttttgtataatgagaaaaagagatttatatacatctcttcttctacttccttcttactagaagatggcaatagaacacaatgagcaacaagaactatctcaatcttttcttttagatgctctttactgtgaagaagaagaagaaaaatggggagatttagtagatgatgagactattattacaccactctcttcagaagtaacaacaacaacaacaacaacaacaaagcctaattctttattacctttgcttttgttggaacaagatttattttgggaagatgaagagcttctttcacttttctctaaagaaaaagaaacccattgttggtttaacagttttcaagatgactctttactctgttctgcccgtgttgattctgtggaatggattttaaaagtgaatggttattatggtttctctgctttgactgccgttttagccataaattactttgacaggtttctgactagtcttcattatcagaaagataaaccttggatgattcaacttgctgctgttacttgtctttctttagctgctaaagttgaagaaactcaagttcctcttcttttagattttcaagtggaggatgctaaatatgtgtttgaggcaaaaactattcaaagaatggagcttttagtgttgtcttcactaaaatggaggatgaatccagtgaccccactttcatttcttgatcatattataaggaggcttgggctaagaaataatattcactgggaatttcttagaagatgtgaaaatctcctcctctctattatggctgattgtagattcgtacgttatatgccgtctgtattggccactgcaattatgcttcacgttattcatcaagttgagccttgtaattctgttgactaccaaaatcaacttcttggggttctcaaaattaacaaggagaaagtgaataattgctttgaactcatatcagaagtgtgttctaagcccatttcacacaaacgcaaatatgagaatcctagtcatagcccaagtggtgtaattgatccaatttacagttcagaaagttcaaatgattcatgggatttggagtcaacatcttcatattttcctgttttcaagaaaagcagagtacaagaacagcaaatgaaattggcatcttcaattagcagagtttttgtggaagctgttggtagtcctcattaaaatcaatcacctgatttatctcttttctttcttattaccaactatggtggtaataatatttattgatattcagaagtatttacctttaatgtcattttcaaaaattacatgaaaatggaaaaaaagaaaagaagagcttagctggtggttgcagttggcagagaagaggactggcttttttttgcaggagtgtagtctactactactggaaagcagagatagagagaggagaaaagacagaaaatctgcactatttgttttttctctattcatatcaattctctcttaggtccttttcatgcatgcatacttttgatggacatattttatatatttactataatcataaattcttgaataaaaaaaaaaaaaaaaaa

SEQ ID NO: 35 - protein, 373 aa, Nicotiana tabacum
MAIEHNEQQELSQSFLLDALYCEEEEEKWGDLVDDETIITPLSSEVTTTTTTTTKPNSLLPLLLLEQDLFWEDEELLSLFSKEKETHCWFNSFQDDSLLCSARVDSVEWILKVNGYYGFSALTAVLAINYFDRFLTSLHYQKDKPWMIQLAAVTCLSLAAKVEETQVPLLLDFQVEDAKYVFEAKTIQRMELLVLSSLKWRMNPVTPLSFLDHIIRRLGLRNNIHWEFLRRCENLLLSIMADCRFVRYMPSVLATAIMLHVIHQVEPCNSVDYQNQLLGVLKINKEKVNNCFELISEVCSKPISHKRKYENPSHSPSGVIDPIYSSESSNDSWDLESTSSYFPVFKKSRVQEQQMKLASSISRVFVEAVGSPH

SEQ ID NO: 36 - DNA, 1430 bp, Nicotiana tabacum
cacctttactctcttctcctttttggctcttcccattctctccttctctttctttattttctgtcctgtagagagagagagaaagtataagcaaagcagcagatatgttactgggtccaagattgagttttggcttaccttgaagataatgagtagagcctccattgtcttcttccgtcaagaagaagaagaagaagatggttttccctttagatactcagctcctaaatccaatctttgatgtcctttactgtgaggaagatcgattcttggacgatgatgatttaggagaatggtctagtactttagaacaagtaggaaataatgtgaaaaagactctacctttattagaatgtgacatgttttgggaagatgaccagcttgtcactcttttaactaaggaaaaagagtctcatttgggttttgattgtttaatctcagatggagatgggtttttagtggaggttagaaaagaggcattggattggatgttgagagtcattgctcactatggtttcactgctatgactgctgttttagctgtgaattattttgataggtttgtatctggactctgctttcagaaagataagccttggatgagtcaacttgctgctgtggcttgtctttctattgctgctaaagtggaagagacccaagtcccccttctcttagacctccaagtggctgattcaagatttgtgtttgaggcaaagactattcagagaatggaactcttggtgctctccactcttaagtggaaaatgaatccagtgacaccactatctttcattgatcatatcatgaggagatttggattcatgaccaatctacatttggattttcttaggagatgtgaacgcctcattcttggtattatcactgattctaggctcttgcattatcctccatctgttattgcaactgcagtagtgtatttcgtgatcaatgagattgagccttgcaatgcaatggaataccagaatcagctcatgactgttcttaaagtcaaacaggatagttttgaagaatgccatgatcttattctagagctaatgggcacttctggctacaatatctgccaaagcctcaagcgcaaacatcaatctgtacctggcagtccaagtggagttatcgatgcatattttagttgcgacagctctaatgattcgtggtcggtagcatcttcaatttcatcgtcaccagaacctcagtataagaggatcaaaactcaggatcagacaatgacactggctccactgagttctgtttctgtcgttgtgggcagtagtcctcgttgatcagtatctcattctctagattatctagtattacggctatggttactatatgatctctcttttttggtatgttctcttaaactgcagttgcacaatgctctgatgttccattaaaaaaaaaaaaaaaaa

SEQ ID NO: 37 - protein, 367 aa, Nicotiana tabacum
MVFPLDTQLLNPIFDVLYCEEDRFLDDDDLGEWSSTLEQVGNNVKKTLPLLECDMFWEDDQLVTLLTKEKESHLGFDCLISDGDGFLVEVRKEALDWMLRVIAHYGFTAMTAVLAVNYFDRFVSGLCFQKDKPWMSQLAAVACLSIAAKVEETQVPLLLDLQVADSRFVFEAKTIQRMELLVLSTLKWKMNPVTPLSFIDHIMRRFGFMTNLHLDFLRRCERLILGIITDSRLLHYPPSVIATAVVYFVINEIEPCNAMEYQNQLMTVLKVKQDSFEECHDLILELMGTSGYNICQSLKRKHQSVPGSPSGVIDAYFSCDSSNDSWSVASSISSSPEPQYKRIKTQDQTMTLAPLSSVSVVVGSSPR

SEQ ID NO: 38 - DNA, 1487 bp, Nicotiana tabacum
gcacgagcttccttcacacaaccagatagagacaagaacagagagattcatggaaatgcccaagaagaatcactcttttggtttcttgttttatataatgagaaagcaacacattttcttcttttctcaaagaagatgggaatacaacacaatgagcataatcaagaccaaacccaatctttccttttagatgctctttactgtgaagaagaaagatgggaagaaacaattgaagatgagattttagaaaaagaagcaacactaccacttcctctgcctttactagaacaagacttgttttgggaagatgaagagctactctctcttttcacaaaagaaaaagaaacaatttccaactttgaaactattaaaacagaccctttactttgtttatctcgtaaagaagctgtgaaatggattcttaaagtaaatgctcattatggattctcaacattcactgctattcttgctattaattactttgataggtttctttcaagtcttcattttcagaaagataagccttggatgattcaacttgtagctgttacttgtctttctttggctgctaaagttgaagaaactcaagttcctcttcttttggacttccaagtggaggatgcaaaatatgtgtttgaggccaaaactattcaaagaatggagcttttggtattgtcctctttaaagtggaggatgaatcctgtaaccccactttcatttgttgatcatataataagaagacttgggctaaagagccatatacactgggaatttctcaagcagtgtgagagaattcttcttttggtcatagctgattgtagattcttaagttatatgccttctgtattggctactgctactatgcttcacgttattcatcaagttgagccttgtaatgctgctgactaccaaaatcaacttcttgaggttctcaacattagcaaggagaaggtgaatgattgctatgaacttataacagaggtgtcttacaactctatttcacacaagcgcaagtatgagagtccaataaatagcccaagtgctgttattgatacattttacagctctgaaaactcaaatgaatcatgggatttgcaaacttcttcctctattccatccacttattcacctcgtgatcaatttttgcctttgtttaagaaaagcagagttcaagaacagcaaatgagattgacatctttaagcagagtttttgtggattatgctgttggcagccctcgctaatattattgacaatgacaatgtattactccttattatgtcctttttgcaaattctactttggaaagatggaaaatgaaaagaggggatagttggtggtagtggctgcagagggttggtgtttgcagcacaatgggggggttaagagaaagaaggataggtaaatattcaatgttctactgggagtaatcgttaattcaaggaacatttagttaattttggtttaagatctctttggttggtccccc

SEQ ID NO: 39 - protein, 368 aa, Nicotiana tabacum
MGIQHNEHNQDQTQSFLLDALYCEEERWEETIEDEILEKEATLPLPLPLLEQDLFWEDEELLSLFTKEKETISNFETIKTDPLLCLSRKEAVKWILKVNAHYGFSTFTAILAINYFDRFLSSLHFQKDKPWMIQLVAVTCLSLAAKVEETQVPLLLDFQVEDAKYVFEAKTIQRMELLVLSSLKWRMNPVTPLSFVDHIIRRLGLKSHIHWEFLKQCERILLLVIADCRFLSYMPSVLATATMLHVIHQVEPCNAADYQNQLLEVLNISKEKVNDCYELITEVSYNSISHKRKYESPINSPSAVIDTFYSSENSNESWDLQTSSSIPSTYSPRDQFLPLFKKSRVQEQQMRLTSLSRVFVDYAVGSPR

SEQ ID NO: 40 - DNA, 1714 bp, Oryza sativa
gtctctctccctccacctccgctcctactctgctgctccaccacgaccaaaagccatgcctatgctgctgctgccgctctcctcctcttccgccattgccgccacctgagctgagctcttgtactcgctacgctactcaagatggctttcgccacgctctttgactccctctactgccccgaggagcacctcgacctcttccatgacaccgccgccgacgacgacctccacctcgaccttcacctgcaccaacccccaccgccgccgccgctcctcgacgacgacctgcctgcgctgttccacgcgctcagggggaaggaggacccgctgcgccccgccgccgacgacgacggctacggcggggtgtctgcccgggaggcggcggtcgggtgggcgctgcgcgccgtcgcgaggctcggcttctccgcgctcacggccgcgctcgccgtcgcctacctcgaccgctgcttcctcggcggcgcgctccgcctcggcgaccgcccctggatggcgcgcctcgccgccgtcgcctgcgtcgcgctcgccgccaaggtggaggagacgcgcgtgcccgtgctcctcgacctccagctctgcgccgccgaacgcgccgaccccaacgaggcctacgtgttcgaggacaagacggtgcgccgcatggagctgctggtgctctcggcgctcggatggcggatgcaccccgtgacgcccctctcctacctccaacccctcctcggcaccgcccacgccgcgcgccttcaccactgcgacaccgcattgctcgcgctgatgcccgattggaggtggcctcgccaccgcccttcggcgtgggccgccgcggcgttgctcgcgacggccggatggtgcggcggcggcggcggcgacgacgccgagctcctagccctcatcgatgcccccaaggatgagatggcagagtgcgccaagatcatctccgaggaggcggcggcggcggcggcggggggcattgtgatcggcggcgaaaataagcgcaagggcgcggcggggctgtactcggcgccggcgagcccgagcggcgtgatcggcgcgtcggcctgcttcagctgcgacagctcgtccagcagcgtcgactcgctgttcgccgccttggagccacccggccggccgatcaagcgaggtgccgccgccgccaccaccgcggatccgcttcccgccgacgaggagagccgcgacgcctggccgccgtacgccgcatgaggctgctctggtcggtatcgggaaggggagaagctaaagcaagccactaattaagcttaggaggaggagggttcagtccaagaaaaagatgttccattaacgcaatgcaagaatgcaggtgcgaaggggtaggttcattcatgtagccattgatggtctcttggagctcagctcagcctcaacctcaacccaccatagccatggatggaggtgaggaagaagctgttttgtcctgtctgctcactctgctggtaccaccactgttagaaccatgcttcacactctctctcttgtttctctctctagagagaagagagagagtataaaggagtaggagtatgaggagtggcaacaatggcaatggcatcaaatgctgcaattgcccacttcccatgagcagctagagctacaacagcagacctgaggctattgtgctcacctcttcttcttccaccattaatgaaaatgcattactacc

SEQ ID NO: 41 - protein, 364 aa, Oryza sativa
MAFATLFDSLYCPEEHLDLFHDTAADDDLHLDLHLHQPPPPPPLLDDDLPALFHALRGKEDPLRPAADDDGYGGVSAREAAVGWALRAVARLGFSALTAALAVAYLDRCFLGGALRLGDRPWMARLAAVACVALAAKVEETRVPVLLDLQLCAAERADPNEAYVFEDKTVRRMELLVLSALGWRMHPVTPLSYLQPLLGTAHAARLHHCDTALLALMPDWRWPRHRPSAWAAAALLATAGWCGGGGGDDAELLALIDAPKDEMAECAKIISEEAAAAAAGGIVIGGENKRKGAAGLYSAPASPSGVIGASACFSCDSSSSSVDSLFAALEPPGRPIKRGAAAATTADPLPADEESRDAWPPYAA

SEQ ID NO: 42 - DNA, 1628 bp, Pisum sativum
gtggcggccgctctagaactagtggatcagaacacaaaaacaaagagaaaaaaaaagatgaatatgaaactcttgattactctccccttctttctataatgaacaaaggaccacacaacctcttcttcattgaagaagatggcaatccatcatcaccaccaccatcatcaacaactacaccacaactctcttcttgatgctctttactgcgatgaagaaaaactcgaagaagaacaagaagacgtttcatctcaacaaagtgatgtcacaacaaacaatgacaacaacatcctagactccacttccctgttccctcttcttctcctggaacaaaacctcttctctcaagatgaagaactcaccacacttttctccaaagaaaaaacccaacaagaaacgtactacgaggatctgaaaaatgtcgtggattttgtttctctctctcaacctcgtcgtgaagctgttcaatggatgcttaaagtcaatgctcattacgccttttcacctctcactgcaacactcgctgttacttactttgataggttccttctaaccttccatttccaaaaagataagccatggatgattcagcttgttgctgttacttgcatctctttagctgctaaagttgaagaaactcaagttcctctcctcttagacctacaagtgcaagatactaaatatgtgtttgaagcaaaaactattcagagaatggagcttttgattctgtcaacactgaaatggaagatgcatcctgtgacaccacactcttttctagatcatataataacaaggcttggtttgaaaactaatcttcattgggagtttttaagacgctgtgagaatcttcttctatctgtacttttagattcaagatttgttggttgtgttccctctgtgttggctactgctacaatgctgcatgtgatagaccagattgaagagagtgatgataatggtgtggactacaaaaatcagcttcttagtattctcaaaatcaacaaggagaaagtggatgaatgttataatgctattgttgaggttactaatgaaaataattatggtcataaacgaaaatatgaacaaatccctggaagtccaagtggcgtaattgatgctgtttttagttctgatggttccaatgattcatggaaagtgggttcatcctcgtattcaacctcagagcctgtttttaagaaaacaaaaactcaagggcaaaataggaatttgtcacctcttaatagggtcattgttggaattcttgccactgctagtgctactacctctccttaatatcctctctctgtcctttataaaaaaaacaaataataaccatatgcaaaaaatctatatttatttagtatatatggttatgttgatgtttctaagatctcttcatttccaacaatgtccctaatgaatctcttgaagaggcagagacagagaaagaggcaagatgaaagaggagtgaaagaggagaggacaaagttggcaatgaagattatgtttttttttttaatttgaaggcactttttatttactattgaatggttgttctcttctagaccataccaaatttgaacatatttatatgatatttctataataaattgggataatttttgttc

SEQ ID NO: 43 - protein, 384 aa, Pisum sativum
MAIHHHHHHHQQLHHNSLLDALYCDEEKLEEEQEDVSSQQSDVTTNNDNNILDSTSLFPLLLLEQNLFSQDEELTTLFSKEKTQQETYYEDLKNVVDFVSLSQPRREAVQWMLKVNAHYAFSPLTATLAVTYFDRFLLTFHFQKDKPWMIQLVAVTCISLAAKVEETQVPLLLDLQVQDTKYVFEAKTIQRMELLILSTLKWKMHPVTPHSFLDHIITRLGLKTNLHWEFLRRCENLLLSVLLDSRFVGCVPSVLATATMLHVIDQIEESDDNGVDYKNQLLSILKINKEKVDECYNAIVEVTNENNYGHKRKYEQIPGSPSGVIDAVFSSDGSNDSWKVGSSSYSTSEPVFKKTKTQGQNRNLSPLNRVIVGILATASATTSP

SEQ ID NO: 44 - DNA, 1491 bp, Populus alba
gagaaagatatgcaatatatgcaaagagaagcctgagagatttttatctccctttttcatttttgatggtgttctataatgagaagaggattaaatctcttcttctctgaagaagatgtctttcttacaacaacaagagactcataatcaaagcccagcattggctcttgacgggctttactgtgaagaggatggatttggagaggattattcttgtggtttggatgatgaaactagccaggtttatgatcaaaatgtgaaaaaggagcaaaatttatcttctgttttgcttgagcaagacttgttttgggaagatagcgagttactgtctttaatctccaaagagaaagagacccatgttgtttttgatagtgtaggatctagagatggatctttaatggtggttcgtagagaggcagttgagtggtttttgagggtaaaggcacattatgggttcagtgctttgactggtgttcttgctgtgaactactttgataggttcatttcaagttcaaggtttcgaagagataagccatggatgggtcaacttgctgctgtggcttgtttgtctctggctgctaaagtggaggaaacccaagtgcctcttcttttagacttgcaagtggaggatgcaaagtacgtttttgaagccaagaccataaagagaatggagctgtgggtgctgtcaactcttcattggaggatgaatcctgtaacctcaatttctttctttgatcacattataaggagacttggattaaagacccacatgcattgggagtttttatggaggtgtgagcgattgcttctttctgtcatttctgattcaaggttcatgagttatcttccttctatattagcaactgcgacaatgttgcatgttatcaaggaggttgagccacgtaatcaactgcaataccaaactcagctcatggctgtgctaaaaaccaatgaggatgaagtgaatgagtgttacaggctcattttagagcaaccaggcagccaaaaccaacgccacaagcgcaagtacctgtccacacccagcagcccaaatggtgtcatcgatgcatctttcagctctgagaactcaaatgattcgtgggctgtggcatcatcaatctcatcatcatcatcagtgcctcaattcaaaagaagcagggcccaggttcagcagatgcgattgccttcactaaatcgtatgtgcgtggatgtgcttagcagtcctcattagtctttccttgtcttaatgcctcgagctatcataatcttcctgttttgcgtattaattgttgtatgtgtaagaggtacagacgttttatgaaactgacccattcttggatgagaaatgaggtatctgctctgctctctctttgttcgagagaggaatgaagagattgtggtgtttttccaataggtatgaacatgatgtctgtatttcaaccattgctctattaaatgttacatgattcactaggaaaaaaaaaaaaaaaa

SEQ ID NO: 45 - protein, 371 aa, Populus alba
MSFLQQQETHNQSPALALDGLYCEEDGFGEDYSCGLDDETSQVYDQNVKKEQNLSSVLLEQDLFWEDSELLSLISKEKETHVVFDSVGSRDGSLMVVRREAVEWFLRVKAHYGFSALTGVLAVNYFDRFISSSRFRRDKPWMGQLAAVACLSLAAKVEETQVPLLLDLQVEDAKYVFEAKTIKRMELWVLSTLHWRMNPVTSISFFDHIIRRLGLKTHMHWEFLWRCERLLLSVISDSRFMSYLPSILATATMLHVIKEVEPRNQLQYQTQLMAVLKTNEDEVNECYRLILEQPGSQNQRHKRKYLSTPSSPNGVIDASFSSENSNDSWAVASSISSSSSVPQFKRSRAQVQQMRLPSLNRMCVDVLSSPH

SEQ ID NO: 46 - DNA, 1405 bp, Populus tremula x Populus tremuloides
ctggaagaagatggcatcaatgtataacccagaaacgagtgcggtacaagaccaacaacaaaaccctacattactttatgatgctctctattgttctgaagagaattgggtggaagaagttagagaggactggtttcaagatgaactagaaggagagagctattgtagcaacaatagcaataaactaaacacttttccaatattgctagaacaggacttaagctgggaagacgaggagctttcctctttgtttgccaaggaggagcaaaatcagctgtgcaaagacttagaaaccaacccgtctttggctagggctcgctgtgaggctgtagagtggattctaaaggtcaatgaacactactctttcaccgctctaactgcagttttggcagtgaactatcttgataggtttttattcagtgtccaccttcagaaagagaagccatggatggcccaacttgcagctgtgtcttgcctctcacttgctgccaaagtggaggagacgcaagtgccccttctattggattttcaggtggaggacagtaaatacgtgttcgaggccaaaactatccagagaatggagatcctggtgctttctactcttaaatggaagatgaatccagtaaccccaatatcgtttcttgattacatcactagaaggcttggcctagaacactatctttgtttggaatttctcaagaggtgtgagcgcatggtcctctctatcttggcagattctaggtctatgccttatgttccttctgtaatggccgctgccacgatgctctatgttattgataacatagaacccagtcttgcagcagaataccaaagccagctgttgagcattcttggaatcgataaagacaaggtagaggattgcagcaagttcttaatggaatttgctctaagagaccattttaagcttctctcaaacaaacgcaagttttgttcacttccaggcagtcctagcggtgtggttgatgtgtcttttagctcagacagctcaaatgattcatggtctgtggcatcatccgtgtcttcatcaccaaagcctctgtccaagaagagtagggcactgcagagtctaaacaacgcaacaacttcagatttttctcagcattcctcgccagtgccttaaaattactgtttttccctaatggaccacctcttgtacggttaataagtcttgtgctttttcaatgttcaagttaaattgcctgccatctctgcttttccagctcagaccaatgcttagaacgctgaaatattgattgggtactgggaattggaagcagagatggttggcattttaccggaaatgaagaacagaacagaacaaaaaaaaagaaaaaaaaagagccgatgaggaagagttctggtagtaaaaaaaaaaaaaaaaaaa

SEQ ID NO: 47 - protein, 376 aa, Populus tremula x Populus tremuloides
MASMYNPETSAVQDQQQNPTLLYDALYCSEENWVEEVREDWFQDELEGESYCSNNSNKLNTFPILLEQDLSWEDEELSSLFAKEEQNQLCKDLETNPSLARARCEAVEWILKVNEHYSFTALTAVLAVNYLDRFLFSVHLQKEKPWMAQLAAVSCLSLAAKVEETQVPLLLDFQVEDSKYVFEAKTIQRMEILVLSTLKWKMNPVTPISFLDYITRRLGLEHYLCLEFLKRCERMVLSILADSRSMPYVPSVMAAATMLYVIDNIEPSLAAEYQSQLLSILGIDKDKVEDCSKFLMEFALRDHFKLLSNKRKFCSLPGSPSGVVDVSFSSDSSNDSWSVASSVSSSPKPLSKKSRALQSLNNATTSDFSQHSSPVP

SEQ ID NO: 48 - DNA, 1152 bp, Arabidopsis thaliana
atggctttagaagaggaggaagagagtcaaaacgcaccgttttgtgttcttgatggtcttttctgtgaggaagagagtgagtttcacgaacaagtagatttgtgcgacgagagtgttgaaaagtttccttttttaaatctgggtttgtctgatcatgatatgttgtgggatgatgatgagttatcaactttgatttcgaaacaagaaccgtgtctttatgacgaaatcttagatgatgagtttctggttttgtgtcgtgaaaaggctcttgattggatttttaaagtgaaatctcattatgggtttaattcattgacggctcttttagctgttaattacttcgataggtttattacaagcaggaagtttcagacagataagccatggatgtctcagcttactgctttggcttgtctgtctttagctgctaaggttgaagagatccgtgttccttttctcttagattttcaagtggaagaagcaagatatgtctttgaagctaagactatacagagaatggagcttcttgttctgtctactcttgactggaggatgcatcctgtgactccaatctcgtttttcgatcacattattcgacgatacagctttaaatctcatcatcaattggagttcttgagtagatgtgaatctttattactctccattattcctgattcgagatttctgagttttagtccttctgtgttagccactgcaataatggtctctgttattagagatttgaagatgtgtgacgaagctgtataccaatctcagctcatgactctactcaaagttgattcggagaaggtaaataaatgctatgagttagtgttagaccacagtccaagcaagaaaaggatgatgaattggatgcaacaacccgctagtccgatcggtgtgtttgatgcgtcattcagttctgatagctcgaatgagtcgtgggttgtgtctgcttctgcttcagtgtcgtcttcaccatcttcagagcctttgctcaagaggagaagagtgcaagagcagcagatgaggctatcttcaataaaccgaatgtttttcgatgtgcttatagtagtcctcgctaaacccagctttcttgtacaaagtggtgatatcacaagcccgggcggtcttctagggataacagggtaa

SEQ ID NO: 49 - protein, 383 aa, Arabidopsis thaliana
MALEEEEESQNAPFCVLDGLFCEEESEFHEQVDLCDESVEKFPFLNLGLSDHDMLWDDDELSTLISKQEPCLYDEILDDEFLVLCREKALDWIFKVKSHYGFNSLTALLAVNYFDRFITSRKFQTDKPWMSQLTALACLSLAAKVEEIRVPFLLDFQVEEARYVFEAKTIQRMELLVLSTLDWRMHPVTPISFFDHIIRRYSFKSHHQLEFLSRCESLLLSIIPDSRFLSFSPSVLATAIMVSVIRDLKMCDEAVYQSQLMTLLKVDSEKVNKCYELVLDHSPSKKRMMNWMQQPASPIGVFDASFSSDSSNESWVVSASASVSSSPSSEPLLKRRRVQEQQMRLSSINRMFFDVLIVVLAKPSFLVQSGDITSPGGLLGITG

SEQ ID NO: 50 - DNA, 1161 bp, Aquilegia formosa x Aquilegia pubescens
atggctcttcaccaccaatatcaacaagaacaacaacaagaatcaacagatccccatttccttgtagactcacttttctgcgaagaagagaaatgggtagaagaagaagaggacttaaatgagagtagtataagtatcatcaataataatggaacaacaacaacaacaacaacaacaacatcatcagtagttgaacttgttccacttttgttgttagaacaagacttgttttgggaagatgaagagcttatctctttgtttagaaaagaacaagatacccatcttgttatatcttctcaacttgattctgatccatctattgctattgctcgtcgtggggttattgattggatgttaagggtcaatgctcattatgctttctctgctctcactgcagttctatctgttaattatcttgatagattcctttcaagttttaagtttcagaaagataaaccatggatgattcaacttgctgctgttgcttgtttatctatagctgctaaagtggaagaaacccaagttcctcttctattagactttcaagttgaagagactatgtatgtgtttgaagcaaaaactattcagagaatggagcttttggtgctttctactcttcattggaagatgaatccagtaacccctctttcttttcttgatcacattataagaaggcttggattgaaaaaccatctacattgggaattttttagaaggtgtgaaggtcttcttttgtctataattgcagattcaaggtttgcttgttttcttccatctgtgttggctacttcaacaatgctgcatgttatagaccaagttgagccttgtaatgcaattgaatatcaaaaccagctaatgggcattcttaaaatcagcaaggataaagtggatgaatgttataaactcatactggaatcaacattgagcttcaataggcatggttacggcaacaaacgcaagtttcaatctatcccaagtagcccaaatggtgtaattgatgcatcattcagttgcgagaattcaaatgattcatgggcattggcttcatctgttacatcatccccagaaccatttttcaagaagagcagagctcaagaccaacagatgagattaccatccttcagtagagtgtttgtggatgttctaagcagtcctccttaa

SEQ ID NO: 51 - protein, 386 aa, Aquilegia formosa x Aquilegia pubescens
MALHHQYQQEQQQESTDPHFLVDSLFCEEEKWVEEEEDLNESSISIINNNGTTTTTTTTTSSVVELVPLLLLEQDLFWEDEELISLFRKEQDTHLVISSQLDSDPSIAIARRGVIDWMLRVNAHYAFSALTAVLSVNYLDRFLSSFKFQKDKPWMIQLAAVACLSIAAKVEETQVPLLLDFQVEETMYVFEAKTIQRMELLVLSTLHWKMNPVTPLSFLDHIIRRLGLKNHLHWEFFRRCEGLLLSIIADSRFACFLPSVLATSTMLHVIDQVEPCNAIEYQNQLMGILKISKDKVDECYKLILESTLSFNRHGYGNKRKFQSIPSSPNGVIDASFSCENSNDSWALASSVTSSPEPFFKKSRAQDQQMRLPSFSRVFVDVLSSPP

SEQ ID NO: 52 - DNA, 1116 bp, Camellia sinensis
atggctcaaccccaaccccaaccccaaccccaaccccaacaacagcaacaaaatcttccatttgttttagatgctctgtattgccaagaacaacatttggagggaggagagacagaggactacttcgattcagaagaggaagattgtcattacagtgataatattgttagccctaagcctccacagctactggaacaagacctgttctgggaagacgaagagttgacctctttgctgtcaaaagaacaagaaaaccccttattccatagtctcgaaacagacccatctttgggtggggctcggagagccgccgtggagtggttgctgaaggtcaacgcccactactcattctctgctctcacggcggtcctcgccgtgaactacctcgataggttcctcttcagcttccatttccagagagagaaaccatggatgacccaacttgctgctgtggcttgtctctctcttgctgccaaagttgaggagactgaagtcccactcctattagacctccaggtggaagatagtaggtatgtttttgaggctaaaacaattcagagaatggagatgctgatactatcaactcttcaatggaagatgaatcctgtgactccactctcatttcttgatcacattacaaggaggttaggtttgaagaacagactttgttgtgaatttctcaagagatgcgagtcaatcctcctctgtatcatttctgattctaggttcatgctttatcttccctccgtattatccactgccacaatgctgctcgtttttagtagtctagagccctgtctcgcagtagaataccaaaaccaactcttgggtattcttcaaatcgacaaggacaaagtggaggattgctataagttaatgctagaatcaacatcaggaattcaccaatccaacaaacgaaagttccgatcaatgccgggcagcccaaattgtgtcacagatgtttgtttcagctccgacagctcgaacgactcgtgggccgtgacatcctcggcatctgcttcggcgtcggtctgttcctcgccggagccattgtcaaagaagagcagagctcaggaccataatgcaactgcagatattctgagcttccattgctag

SEQ ID NO: 53 - protein, 371 aa, Camellia sinensis
MAQPQPQPQPQPQQQQQNLPFVLDALYCQEQHLEGGETEDYFDSEEEDCHYSDNIVSPKPPQLLEQDLFWEDEELTSLLSKEQENPLFHSLETDPSLGGARRAAVEWLLKVNAHYSFSALTAVLAVNYLDRFLFSFHFQREKPWMTQLAAVACLSLAAKVEETEVPLLLDLQVEDSRYVFEAKTIQRMEMLILSTLQWKMNPVTPLSFLDHITRRLGLKNRLCCEFLKRCESILLCIISDSRFMLYLPSVLSTATMLLVFSSLEPCLAVEYQNQLLGILQIDKDKVEDCYKLMLESTSGIHQSNKRKFRSMPGSPNCVTDVCFSSDSSNDSWAVTSSASASASVCSSPEPLSKKSRAQDHNATADILSFHC

SEQ ID NO: 54 - DNA, 1119 bp, Camellia sinensis
atgaagaggatgtctccttacccagagcaagactcacatctacagaacccaatgtttgtctttgacggtctctactgtgaagaagagcattttgaggatgatttgggagagtatggtttggaacaagggagtgacaactgcgatgagaatgtgaaaggacctttagttttcttggaacatgactgggattgggatgatgatgagcttgtttctttaatttccaaagagaaagagacccatttgggtctgagtgttttgaactcagacgagtccttaatggtggcaaggagagaatctgttgattggattttaagggtcattgctcactatggtttcactgttttgaccactgttttagcagttaactactttgatagattcatttcaagcctttcatttcagagagagaagccatggatgagtcaacttgttgctgttgcttgtctctctttagctgccaaagttgaggagacccaagtgccccttctcttagacttccaagtggaggaatcaaagtttgtgtttgaagccaagacaatccagagaatggagcttctggtgctatctactcttcaatggaagatgaatcctgtgaccccactttcatttgttgatcacattgtgaggaggtttggatttaagacaaatttgcatttggagtttctgtggaggtgtgagcgccttcttctctctgccatcactgattcaaggtttgggtgttatcttccttctgtattggctgctgcaacaatgttacatgttatcaaagaggttgagccttctaatgtattggactgtcaaaatgagcttatggatgttctcaaaatgagcaaggacaaagtagatgattgctacaaactcatcctcgaactgcctggcaacaacagtcaaatgcaatgccaaacccacaagcgcaagtatcagtccatacccaacagcccaaatggtgtcatcgatgtgaatttcagctgcgatagctcgaacgattcttgggcagtgacatcctcagtttcatcatcaccagaacccctgttcaagaagagcagagttcatggtcagcagatgagattggctccattaaggcatatgtctgtgggtgtagttggcagccctcgttaa

SEQ ID NO: 55 - protein, 372 aa, Camellia sinensis
MKRMSPYPEQDSHLQNPMFVFDGLYCEEEHFEDDLGEYGLEQGSDNCDENVKGPLVFLEHDWDWDDDELVSLISKEKETHLGLSVLNSDESLMVARRESVDWILRVIAHYGFTVLTTVLAVNYFDRFISSLSFQREKPWMSQLVAVACLSLAAKVEETQVPLLLDFQVEESKFVFEAKTIQRMELLVLSTLQWKMNPVTPLSFVDHIVRRFGFKTNLHLEFLWRCERLLLSAITDSRFGCYLPSVLAAATMLHVIKEVEPSNVLDCQNELMDVLKMSKDKVDDCYKLILELPGNNSQMQCQTHKRKYQSIPNSPNGVIDVNFSCDSSNDSWAVTSSVSSSPEPLFKKSRVHGQQMRLAPLRHMSVGVVGSPR

SEQ ID NO: 56 - DNA, 1128 bp, Citrus sinensis
atggcatttggagatgaacaatacccttcttcattcttgcttgatgcactctattgtgaagaagaagagttagaagatgaggttattgaccaagaagatgatgaatgtagccaaaacaaaaacccagcttgtttgttttcacttcttttgttagaacaagacttgttctgggaagatgaagagctcttgtctctcttctccaaagaagagcaacagcttttaaagcaagaaacacaaacccattataaagattccgatgttcttgttgttgctaggagtgaggctgttgagtgggtgctcaaagttaatgctcattatgggttctctactctcactgcaatactggctattaactatctggataggttcctccgtagcttccattttcaaatagataagccttggatgattcagcttttggctgtcacttgtctctccctggctgctaaagttgaagaaacccaagtgccccttctcttagaccttcaagttgagggggcaaaatatgtttttgagaccaaagccatacaaagaatggagcttttggtgctctcaacacttgaatggaagatgcatccagtgactccaatttcatttcttgaccacatcataagaaggcttggattgaagacatctcttcactgggagtttctcaagagatgtgagcgtctgcttctcactttggtctctgattcaagatctgtaagttaccttccttcagtgttggccactgccacaatgatgcacataatagaccaagttgagcctgtgaatcccgttgattatcaaaaccagcttctaggtgtgcttaaaataagcaaggaaaaagtaagtgactgttacaagttgattcttgagctggctaatgcaaaaaccaatgctaatagtaatcctcacaagcgcaagtttgaagcaatccctggaagccctggtggcgtgattgatgctactgtgtttagctgtgatgaaagctcaaacgattcatggtcagtggcatcatcatcagtcctatcatcaccatcgtcaccagagcctctcttcaaaaagagcagagtccaagacccacaaatgactttgccaatgccatctctcaatctcaatagggtcattgtgggcagtccaagttga

SEQ ID NO: 57 - protein, 375 aa, Citrus sinensis
MAFGDEQYPSSFLLDALYCEEEELEDEVIDQEDDECSQNKNPACLFSLLLLEQDLFWEDEELLSLFSKEEQQLLKQETQTHYKDSDVLVVARSEAVEWVLKVNAHYGFSTLTAILAINYLDRFLRSFHFQIDKPWMIQLLAVTCLSLAAKVEETQVPLLLDLQVEGAKYVFETKAIQRMELLVLSTLEWKMHPVTPISFLDHIIRRLGLKTSLHWEFLKRCERLLLTLVSDSRSVSYLPSVLATATMMHIIDQVEPVNPVDYQNQLLGVLKISKEKVSDCYKLILELANAKTNANSNPHKRKFEAIPGSPGGVIDATVFSCDESSNDSWSVASSSVLSSPSSPEPLFKKSRVQDPQMTLPMPSLNLNRVIVGSPS

SEQ ID NO: 58 - DNA, 1191 bp, Glycine max
  misc_feature (117)..(117): n is a, c, g, or t
atggcaattcagcaccacaatgaacaactagagcataatgaaaatgtctcatctgtccttgatgccctttactgcgatgaaggaaagtgggaggatgaagaggaggaggaagaagantatgaagaaagtgaagtaacaacaaacactggaacttctcttttccctctgctcatgttggagcaagacttgttctgggaagatgaggaactaaactctctcttttccaaagagaaggttcaacatgaagaagcctatgactataacaatctgaacagtgatgataatagcaatgatcacagtaataataacaataatgtgctgtcggactcttgtctctctcagcctcgtcgtgaggcagtggaatggatactgaaagtcaatgctcactatggattctctgctctcactgcaacactggccgttacttacctggataggttccttctaagcttccattttcaaagggagaagccatggatgatccagcttgtggctgtcacttgcatctctttggctgcaaaagttgaagaaactcaagtgcctcttctcttggaccttcaagtgcaagacacaaagtatgtgtttgaggcaaagactattcagagaatggagctcctggtgctgtccaccctcaaatggaagatgcaccccgtgacacccctctcctttctagatcacattataagaaggcttggattgaaaacacatcttcactgggagtttctcaggcgctgtgagcatcttcttttgtctgtgcttttagattcaagatttgttggttgtcttccttctgtgttggccactgcaacaatgctgcatgttatagaccagattaaacacaatggtgggatggaatacaaaactcagcttctgagtgttctcaaaattagcaaggagaaagtagatgagtgttataatgcaattctccaactctcaaaggccaataaatatggtcataacaacatcaacaacactagcaagcgcaagtatgagcaaatcccaagcagcccaagtggcgtaattgatgctgcattttgctctgatggttccaacgattcgtgggcagtggggtcatcattgttatattcaccaccagagcctctcttcaagaagagcagaacccaaggacaacaaatgaatttgtcaccacttaaacggttcattatcggaattgttggcacccctccttaa

SEQ ID NO: 59 - protein, 396 aa, Glycine max
  misc_feature (39)..(39): Xaa can be any naturally occurring amino acid
MAIQHHNEQLEHNENVSSVLDALYCDEGKWEDEEEEEEXYEESEVTTNTGTSLFPLLMLEQDLFWEDEELNSLFSKEKVQHEEAYDYNNLNSDDNSNDHSNNNNNVLSDSCLSQPRREAVEWILKVNAHYGFSALTATLAVTYLDRFLLSFHFQREKPWMIQLVAVTCISLAAKVEETQVPLLLDLQVQDTKYVFEAKTIQRMELLVLSTLKWKMHPVTPLSFLDHIIRRLGLKTHLHWEFLRRCEHLLLSVLLDSRFVGCLPSVLATATMLHVIDQIKHNGGMEYKTQLLSVLKISKEKVDECYNAILQLSKANKYGHNNINNTSKRKYEQIPSSPSGVIDAAFCSDGSNDSWAVGSSLLYSPPEPLFKKSRTQGQQMNLSPLKRFIIGIVGTPP

SEQ ID NO: 60 - DNA, 1125 bp, Gossypium hirsutum
atggcaatacagcaatatgaacagcagcaacaacaaccagagaatcacccttccttcttgctagatgctctctactgtgaggaagaagcggatgcaggggaagttttagaggaagagagttcttgtgtgggctgtaacaatggcggaaacccttcatttttcccactgttgttgttagagcaggatttgttttgggaagacggggagcttctttcactttttgctaaagaaacagagcagcagccgtcttgtttcaatgtgggaaccgatgagtccctagcaatggctcgccgagaggctgccgagtggatgcttaaagtcaatgctcgatttggattctccactctcacggctgtactttccattaactatttggacaggttcttaagtacctttcagtttcaaagagataatccttggatgatccaacttctgggtgtcacttgtctctctttggctgcaaaagttgaagagacacaagtgcctctgctcctagacctacaagtggaggagacaaagtatgttttcgaggccaaaactatccaaagaatggagcttttggtgctctccacactgaaatggaagatgcatccaattacacccctttcatttctagatcacatcataagaagactggggttgaaaacccacctccattgggagtttcttaagcgatgtgagcgtctcctcctctgtgtaatctctgatgcaagatccatccattatcttccctctgtattggctactgcaaccatgatgcacgtcatagaccaagttgagcttttcaatcccattgactaccaaaatcagctgctgagtgttcttaaaattagcaaggaaaaagtaaacgattgttacaagctcatccttgatgtatcaacaagaccccaggcccaaggcaatggtggtgcatgtaagaggaaggtggaggagagggttcctagcagccctagtggagtgattgatgctgcatttggcagtgatagctcgagcgattcttggggcacggtgtccttatcgcctgagcagcagccaccttttaagaagagcagagcccaagagcaagtaatgcgtttgccatcactcaaccgagtctttgtagacattgttggcagcccttcttaa

SEQ ID NO: 61 - protein, 374 aa, Gossypium hirsutum
MAIQQYEQQQQQPENHPSFLLDALYCEEEADAGEVLEEESSCVGCNNGGNPSFFPLLLLEQDLFWEDGELLSLFAKETEQQPSCFNVGTDESLAMARREAAEWMLKVNARFGFSTLTAVLSINYLDRFLSTFQFQRDNPWMIQLLGVTCLSLAAKVEETQVPLLLDLQVEETKYVFEAKTIQRMELLVLSTLKWKMHPITPLSFLDHIIRRLGLKTHLHWEFLKRCERLLLCVISDARSIHYLPSVLATATMMHVIDQVELFNPIDYQNQLLSVLKISKEKVNDCYKLILDVSTRPQAQGNGGACKRKVEERVPSSPSGVIDAAFGSDSSSDSWGTVSLSPEQQPPFKKSRAQEQVMRLPSLNRVFVDIVGSPS

SEQ ID NO: 62 - DNA, 1173 bp, Lotus corniculatus
atggcaatccatcaacatcatcacaacaatgtcattgaccagctagaacaaaatgaaaatgtttcttctgtcttggatgctctttactgtgatgaagaaaaatgggaggaagaggaagtagaacaagtggttggagagttatctgaagaagaaacaagtgatgtgacaacaaacaatgaccctaacaacacttgttctctgtttcccctgcttttgttggagcaagacttgttctgggaagatgaagaactcaactctctcttctccaaagagaagatccaacaccaaaactattataatgatgtgaactcggacccttttctctctcagcctcgtcatgaggcagtgaaatggatgcttaaagtcaatgctcattatggattctctgctctcactgcaacacttgctgttacctactttgataacttccttttgagcttccattttcaaagtgagaagccatggatgatccagcttgctgctgttacttgcatctctttggcagctaaagttgaagaaacccaagtgccacttctcttagaccttcaagtgcaagatgctaagtttgtgtttgaggcaaagaccattctgaaaatggagcttctggttctgtccacactcaaatggaagatgcatcctgtgactccactttcatttctggatcacattatcagaaggcttggattgaaaacacaccttcattgggagtttctcaggcgctgtgagcatcttcttttgtctgtgcttttagattcaagatttgttggtgttcttccttctgtgttggccactgcaacaatgctgcatgttatagaccagattgagaagagtgatggggtggaatacaaaaagcagcttctgggtgttctcaaaattaacaaggggaaagtagatgaatgctatgatgccatgcttgagcttacaaatgccaatgattatgatgataacaagaagcttaataagcgcaagtatgaggaaataatccctggtagcccaagtggcgtcattgatgccgcatttaactctgatggttccaacgattcgtggacagtggggtcatcattgttttcatcctcaggcccagagtctcctctgttcaagaaaagcagaacccaaatgaaattgtcaccacttaacagggtcattgttggaattgttagcacttcaccttga

SEQ ID NO: 63 - protein, 390 aa, Lotus corniculatus
MAIHQHHHNNVIDQLEQNENVSSVLDALYCDEEKWEEEEVEQVVGELSEEETSDVTTNNDPNNTCSLFPLLLLEQDLFWEDEELNSLFSKEKIQHQNYYNDVNSDPFLSQPRHEAVKWMLKVNAHYGFSALTATLAVTYFDNFLLSFHFQSEKPWMIQLAAVTCISLAAKVEETQVPLLLDLQVQDAKFVFEAKTILKMELLVLSTLKWKMHPVTPLSFLDHIIRRLGLKTHLHWEFLRRCEHLLLSVLLDSRFVGVLPSVLATATMLHVIDQIEKSDGVEYKKQLLGVLKINKGKVDECYDAMLELTNANDYDDNKKLNKRKYEEIIPGSPSGVIDAAFNSDGSNDSWTVGSSLFSSSGPESPLFKKSRTQMKLSPLNRVIVGIVSTSP

SEQ ID NO: 64 - DNA, 1161 bp, Medicago trunculata
atggctatccatcatcatcatcacaatcaccaacaacttcaacaacacacttcttctctttttgatgcactttactgtgatgaagaagaaaaatgggaagatgatgatgaaggagaagttgtagatgaaggagcacaaagtgatgtcacaacaacaaactatgatatattggactctacttcccttttacctctgcttttgttagaacagaacttgttcaatgaagatgaagaactcaacactcttttctccaaagagataactcaacaagaaacatattacgaggatctgaaaaatgtgatcaactttgactcactctctcaaccacgtcgtgaagctgttgaatggatgcttaaagtcaatgctcattatggtttctctgctctcactgcaacacttgctgttaactatcttgataggtttcttttaagcttccatttccaaaaagagaaaccatggatgattcagcttgttgctgttacttgcatctctttagctgctaaagttgaagaaactcaagttcctcttctcttagaccttcaagtgcaagatactaaatatgtgtttgaggcaaagactattcagagaatggagctattgattctgtcaacactgaaatggaagatgcatccagtgacaacacactcttttctagatcacattataagaaggcttggattgaaaactaatcttcattgggagtttcttaggcgctgtgagaatcttcttctatctgtacttttagattcaagatttgttggttgtgttccttctgtgttggccactgctacaatgttgcatgttatagaccagattgaacagagtgatgataatggtgtggattacaaaaatcagcttcttaatgttctcaaaatcagcaaggagaaagttgatgaatgttataatgcgattcttcatcttacaaatgcaaataattatggtcataaacgaaaatatgaagaaatccctggtagtccaagtggcgtaattgatgctgtttttagttctgatggttctaacgattcgtggacagtgggagcatcatcatattcaacctcagagcctgtgtttaagaagaccaagaatcaaggacaaaatatgaatttgtcaccgattaacagggtcattgtcggaattcttgccactgcaacctctccttaa

SEQ ID NO: 65 - protein, 386 aa, Medicago trunculata; identical to SEQ ID NO: 33

SEQ ID NO: 66 - DNA, 1119 bp, Scutellaria baicalensis
atggtttcggaatttcaggagcacgaatcccttctccaaaaccctatctttgatgccctttattgtgacgaggagcgttttgatgaatgtgtaggcggcgctggttcgggcttcaaagagcccgaaatcaacgattttaatgagattcacaataacccttttgcttttctgtttgagcacgaccttttctgggagagtgaggagcttgacgccctgttaacgaaggagaaaacgcagacccatttgacttttgatgaaataaactcagatgcgtctttgaaggcgatgagaaatgaggcgattaactggatgctgaaggtgattgcccactacggcttcaatgcgctgactgctgttttggctgtcaactattatgatagattcatcactagtgtttgttttcagaaggataagccatggatgagtcaattagctgctgtggcttgtctttctgtagctgccaaggtggaggaaactcaagtgcctcttttgttggatttacaagttgaagaatctaagtatttgtttgaagctaagaccatccaaagaatggagcttttggtgctttctaccctccaatggaggatgaatcctgtgacgccaatctcattctttgaccacattgcaaggagatttgagtttgtaaagaacctacattctgtatttttaaggaggtgtgagagtttaatcctctccattatcactgattgtagattggtaaagtattttccttcagttattgcttctgcagcaatgatatatgcgattagagagtttgagactcctgatgctctggaatatgaggatcaactcttgagtgtgctaagaactagcaaggacaaagttgatgattgccgcaaactcattgtggatgcaatgtatggtggtttcagccacaagccttgctataaacgcaaatatgagtcgatcccaagcagtccaagtggtgtcattgatgcgtatttgagctctgatagctctgttgattcgtgggctgttacattatcagtgtcatcgtcgccagagccttcgtttaagagaagcaaagctcaagatcagcacatgagattggctccactaagcagtgtatctcttggccttgctcatcgtattaattga

SEQ ID NO: 67 - protein, 372 aa, Scutellaria baicalensis
MVSEFQEHESLLQNPIFDALYCDEERFDECVGGAGSGFKEPEINDFNEIHNNPFAFLFEHDLFWESEELDALLTKEKTQTHLTFDEINSDASLKAMRNEAINWMLKVIAHYGFNALTAVLAVNYYDRFITSVCFQKDKPWMSQLAAVACLSVAAKVEETQVPLLLDLQVEESKYLFEAKTIQRMELLVLSTLQWRMNPVTPISFFDHIARRFEFVKNLHSVFLRRCESLILSIITDCRLVKYFPSVIASAAMIYAIREFETPDALEYEDQLLSVLRTSKDKVDDCRKLIVDAMYGGFSHKPCYKRKYESIPSSPSGVIDAYLSSDSSVDSWAVTLSVSSSPEPSFKRSKAQDQHMRLAPLSSVSLGLAHRIN

SEQ ID NO: 68 - DNA, 1164 bp, Zea mays
atggcagctttcgccgcgttgttcgaccccctctactgcccggaggagcacctcgatctgtaccacgaaggacccgtcgaggttgtggacgagcagtggcaggaccagcgcggacagcagcaaccggcggctcttgacgacgagctgccggcgctgttcgaggcgctccgggacaaggagggggtggtgctggcgggtgatggggaggaggatgggtacggcggctcggcaggccgggaggccgcagtcggctgggcgtcacgcgccgcggcacggctgggcttctctgcgctcacttccgcgctgtccgccgcctacctggaccgctgcttcctccccgggggcgcgctccgtctcggcgaccagccctggatgtcgcgcctcgccgccgtcgcctgtgtcgcgctcgccgccaaggtcgaggaaacgcgcgtgccgctgctcctcgacctccagctctgcgccgccgccagctccgacgctgacgcagcggacgcggacgtgttcgaggccaagacggtgcgccggatggagctgctcgttctctccgcgctagggtggcggatgcaccctgtcacgcccttctcctacctccagcctgtcctcgccgacgctgcgatgcgcctacgcaactgcgaggccgtcctgctcgcggtcatggccgattggaggtggcctcggcaccggccctcggcgtgggccgccgccgcattgctcaccacagccggcggcggcgacgacgactcggagctgctcgcgctcatcaatgcccccgaggacgagaccgcggagtgcgccaagatcatctccgaggtgacaggcatgagcttccttgtctgcgacgtcggcggcatgatcgccgggaataagcgtaagcacgcggcggcgcggatgtactcgccgccgctgagcccgagcggcgtgatcggcgcgctgtcctgcttcagctgcgagagctcgttgtccgccacagcggactcgcgcaccctcgctactacggctgcgggggtcggcccgtgggcaccgtcagcgcccgtgtccgtgtcgtcttcccctgagcccccaggtcgggcccccaagcgcgctgcggcggcgggggtcccgcatccgcttccccccgacgaggagagccgcgacgcctggccgtccacctgcgccgcgtga

SEQ ID NO: 69 - protein, 387 aa, Zea mays
MAAFAALFDPLYCPEEHLDLYHEGPVEVVDEQWQDQRGQQQPAALDDELPALFEALRDKEGVVLAGDGEEDGYGGSAGREAAVGWASRAAARLGFSALTSALSAAYLDRCFLPGGALRLGDQPWMSRLAAVACVALAAKVEETRVPLLLDLQLCAAASSDADAADADVFEAKTVRRMELLVLSALGWRMHPVTPFSYLQPVLADAAMRLRNCEAVLLAVMADWRWPRHRPSAWAAAALLTTAGGGDDDSELLALINAPEDETAECAKIISEVTGMSFLVCDVGGMIAGNKRKHAAARMYSPPLSPSGVIGALSCFSCESSLSATADSRTLATTAAGVGPWAPSAPVSVSSSPEPPGRAPKRAAAAGVPHPLPPDEESRDAWPSTCAA

SEQ ID NO: 70 - DNA, 1176 bp, Zea mays
atggcagctttcgccgcgctgttcgaccccctgtactgcccggaggagcacctcgatctgtaccgcgacgaacccggcgagggtgcggacgagcagtggccgggccagcacggacagcaggagccggctgtcctcgacgacgagctgccggcgctgttcgaggcacaccgggccaaggagggggtggtgctggcggaggatggcgggtacggcggcgcagctgggcgtgaggccgcggtcggctgggtttcacgcgccgcggcgcggctaggcttctccgcgctcaccgccgcgctcgccgccgcctacctcgaccgctgcttcctccccgggggcgcgctccggctcggcgaccagccctggatggcgcgcctagccgccgtcacctgcttcgcgctcgccgccaaggtcgaggagacgcgcgtgccgccgctcctcgacctccagctctacgccgccgctgacgccgcggatccgtacgtattcgaggccaagacggtgcgccggatggagctgctcgtgctctccgcgcttgggtggcggatgcaccctgtcacgcccttctcctacctccagcccgtcctcgccgacgctgcgacgcgcctgcgtagctgcgagggcgtcctgctcgcggtcatggccgactggaggtggcctcggcaccggccttcggcgtgggccgccgccgcgttgctgatcacagccgccgccggcgacggcggcgacggcgacggcgacacggagctcctggcgctcctcattgcccccgaggacaagaccgccgagtgtgccaagatcatctccgaggtgacgggcatgagcttcctcgcctgcgatgtcggcgtgagcgccggaaataagcgtaagcacgcggcggcgcagttgtactcgccgccgccgagcccgagcggcgtgatcggcgcgctgtcctgcttcagctgcgagagctcgacgtccgccaccgctatggctgcggcggtcggcccgtgggcgccgtcggcgtccgtgtccgtgtcgtcctctccagagccaccaggtcgggcccccaagcgcgcagcggcggcgtcggcgtcggcgtcggcgtcagccggggtcgcgccaccggtccaggtcccgcatcagctaccccccgacgaggagagccgcgacgcctggccgtccacctgcgccgcgtga

SEQ ID NO: 71 - protein, 391 aa, Zea mays
MAAFAALFDPLYCPEEHLDLYRDEPGEGADEQWPGQHGQQEPAVLDDELPALFEAHRAKEGVVLAEDGGYGGAAGREAAVGWVSRAAARLGFSALTAALAAAYLDRCFLPGGALRLGDQPWMARLAAVTCFALAAKVEETRVPPLLDLQLYAAADAADPYVFEAKTVRRMELLVLSALGWRMHPVTPFSYLQPVLADAATRLRSCEGVLLAVMADWRWPRHRPSAWAAAALLITAAAGDGGDGDGDTELLALLIAPEDKTAECAKIISEVTGMSFLACDVGVSAGNKRKHAAAQLYSPPPSPSGVIGALSCFSCESSTSATAMAAAVGPWAPSASVSVSSSPEPPGRAPKRAAAASASASASAGVAPPVQVPHQLPPDEESRDAWPSTCAA

SEQ ID NO: 72 - protein, 343 aa, Chenopodium rubrum
MTTLETEQEQQPFSQNSPLFLDCLYCEEKYWDYDYHDEDDFGSLNSSKLHDCSLICCEDDEEIQLNALVSKEEKINFDEGDLGGNQLVMETRREALEWMIRVNYHHNFSVITLVLGVNYFDRFMLSFGFQKEMPWMTHLAAVACLSLASKVEETHVPLLLDFQVEHEQIFEAKVVQRMELLVLQHSNGKMNAVTPLSYFGHLIRKLKLKPHFHCKILTRCENIIVSVILDPRFLCYVPSVLAAASMVQTLKEIGLWSILEHQNDIMNTLKLDKVKVEDCYNFIQEVSSNEKARKRKWYNNISSANRNPNNVLELVVSSESSNNDLPSETLPKKCRTMGPPCFG

SEQ ID NO: 73 - protein, 335 aa, Arabidopsis thaliana
MRSYRFSDYLHMSVSFSNDMDLFCGEDSGVFSGESTVDFSSSEVDSWPGDSIACFIEDERHFVPGHDYLSRFQTRSLDASAREDSVAWILKVQAYYNFQPLTAYLAVNYMDRFLYARRLPETSGWPMQLLAVACLSLAAKMEEILVPSLFDFQVAGVKYLFEAKTIKRMELLVLSVLDWRLRSVTPFDFISFFAYKIDPSGTFLGFFISHATEIILSNIKEASFLEYWPSSIAAAAILCVANELPSLSSVVNPHESPETWCDGLSKEKIVRCYRLMKAMAIENNRLNTPKVIAKLRVSVRASSTLTRPSDESSSPCKRRKLSGYSWVGDETSTSN

SEQ ID NO: 74 - protein, 361 aa, Arabidopsis thaliana
MAENLACGETSESWIIDNDDDDINYGGGFTNEIDYNHQLFAKDDNFGGNGSIPMMGSSSSSLSEDRIKEMLVREIEFCPGTDYVKRLLSGDLDLSVRNQALDWILKVCAHYHFGHLCICLSMNYLDRFLTSYELPKDKDWAAQLLAVSCLSLASKMEETDVPHIVDLQVEDPKFVFEAKTIKRMELLVVTTLNWRLQALTPFSFIDYFVDKISGHVSENLIYRSSRFILNTTKAIEFLDFRPSEIAAAAAVSVSISGETECIDEEKALSSLIYVKQERVKRCLNLMRSLTGEENVRGTSLSQEQARVAVRAVPASPVGVLEATCLSYRSEERTVESCTNSSQSSPDNNNNNNNSNKRRRKQ

SEQ ID NO: 75 - protein, 308 aa, Arabidopsis thaliana
MAEENLELSLLCTESNVDDEGMIVDETPIEISIPQMGFSQSESEEIIMEMVEKEKQHLPSDDYIKRLRSGDLDLNVGRRDALNWIWKACEVHQFGPLCFCLAMNYLDRFLSVHDLPSGKGWILQLLAVACLSLAAKIEETEVPMLIDLQVGDPQFVFEAKSVQRMELLVLNKLKWRLRAITPCSYIRYFLRKMSKCDQEPSNTLISRSLQVIASTTKGIDFLEFRPSEAAAAVALSVSGELQRVHFDNSSFSPLFSLLQKERVKKIGEMIESDGSDLCSQTPNGVLEVSACCFSFKTHDSSSSYTHLS

SEQ ID NO: 76 - protein, 260 aa, Arabidopsis thaliana
MAEFMEPNLVSNFDDEKSNSVDTRSIFQMGFPLESEEIVREMIEKERQHSPRDDYLKRLRNGDLDFNVRIQALGWIWKACEELQFGPLCICLAMNYLDRFLSVHDLPSGKAWTVQLLAVACLSLAAKIEETNVPELMQLQVGAPMFVFEAKSVQRMELLVLNVLRWRLRAVTPCSYVRYFLSKINGYDQEPHSRLVTRSLQVIASTTKGIDFLEFRASEIAAAVALSVSGEHFDKFSFSSSFSSLEKVREVTKSLLHLQK

SEQ ID NO: 77 - protein, 323 aa, Arabidopsis thaliana
MGEPKDSLALFLCHESESSLNEDDDETIERSDKQEPHFTTTIDDEDYVADLVLKENLRFETLPSKTTSSSDRLIAIDWILTTRTRFGFQHQTAYIAISYFDLFLHKRFIGLQKDETWAMRLLSVACLSLAAKMEERIVPGLSQYPQDHDFVFKPDVIRKTELLILSTLDWKMNLITPFHYFNYFLAKISQDNHSVSKDLVLLRSSDSLLALTKEISFTEYRQFVVAAVTTLLASSSTSSDIRLTREEIANKFGSISWWTSNENENVYLCYQRTLEIEERKHMTPPPEIAVSREPPASGSGAKRRLSFDDSDQSSPPAKRMRRL

SEQ ID NO: 78 - protein, 302 aa, Arabidopsis thaliana
MEFHLEHPLSHSSLHNNFNDDTDYETLPHSLFLVEFQHMPSSHYFHSLKSSAFLLSNRNQAISSITQYSRKFDDPSLTYLAVNYLDRFLSSEDMPQSKPWILKLISLSCVSLSAKMRKPDMSVSDLPVEGEFFDAQMIERMENVILGALKWRMRSVTPFSFLAFFISLFELKEEDPLLLKHSLKSQTSDLTFSLQHDISFLEFKPSVIAGAALLFASFELCPLQFPCFSNRINQCTYVNKDELMECYKAIQERDIIVGENEGSTETAVNVLDQQFSSCESDKSITITASSSPKRRKTSTRRY

SEQ ID NO: 79 - protein, 341 aa, Arabidopsis thaliana
MDNLLCEESWPASPLTPEPLPNFRHRSHDNDVVKMYPEIDAATMEEAIAMDLEKELCFNNHGDKFVEFFVSKKLTDYRFHAFQWLIQTRSRLNLSYETVFSAANCFDRFVYMTCCDEWTNWMVELVAVTSLSIASKFNEVTTPLLEELEMEGLTHMFHVNTVAQMELIILKALEWRVNAVTSYTFSQTLVSKIGMVGDHMIMNRITNHLLDVICDLKMLQYPPSVVATAAIWILMEDKVCRESIMNLFEQNHKEKIVKCVDGMKNRDIDHQSSRRRYSEGRSILSLLQRGDVMNMNGDYNVEDLSKIFQIFRYEKKKRDRGNHQDNIRPAKRMTIEMSNYI

SEQ ID NO: 80 - protein, 315 aa, Helianthus tuberosus
MSISCSDCFSDLLCCEDSGILSGDDRPECSYDFEYSGDFDDSIAEFIEQERKFVPGIDYVERFQSQVLDASAREESVAWILKVQRFYGFQPLTAYLSVNYLDRFIYCRGFPVANGWPLQLLSVACLSLAAKMEETLIPSILDLQVEGAKYIFEPKTIRRMEFLVLSVLDWRLRSVTPFSFIGFFSHKIDPSGMYTGFLISRATQIILSNIQEASLLEYWPSCIAAATILCAASDLSKFSLINADHAESWCDGLSKEKITKCYRLVQSPKILPVHVRVMTARVSTESGDSSSSSSSPSPYKKRKLNNYSWIEEDKR

SEQ ID NO: 81 - protein, 359 aa, Medicago sativa
MIEEEQLPAILMPSDCELLCGEDSSEVLTGDLPECSSDLDSSSSSQLPSSSLFAEEEEDSIAVFIEHEFKFVPGFDYVSRFQSRSLESSTREEAIAWILKVHEYYGFQPLTAYLSVNYMDRFLDSRPLPGIKWMATATFICCMFVFSSKDGGPLVPSLLDFQIEGAKYIFQPRTMLIMELLVLTILDWRLRSITHLVSSVSLRATRFTGTFNHFIISRATEIILSNIRDASFLTYRPSCIAAAAILSAANEIPNWSFVNPEHAESWCEGLSKEKIIGCYELIQEIVSSNNRRNAPKVLPQLRVTARTTRWSTVSSLSSSPSSSSSPSYSLSYKKRKLNSCFWVDVDKGNSEGREKKQTR

SEQ ID NO: 82 - protein, 354 aa, Nicotiana tabacum
MAADNIYDFVASNLLCTETKSLCFDDVDSLTISQQNIETKSKDLSFNNGIRSEPLIDLPSLSEECLSFMVQREMEFLPKDDYVERLRSGDLDLSVRKEALDWILKAHMHYGFGELSFCLSINYLDRFLSLYELPRSKTWTVQLLAVACLSLAAKMEEINVPLTVDLQVGDPKFVFEGKTIQRMELLVLSTLKWRMQAYTPYTFIDYFMRKMNGDQIPSRPLISGSMQLILSIIRSIDFLEFRSSEIAASVAMSVSGEIQAKDIDKAMPCFFIHLDKGRVQKCVELIQDLTTATITTAAAASLVPQSPIGVLEAAACLSYKSGDERTVGSCTTSSHTKRRKLDTSSLEHGTSEKL

SEQ ID NO: 83 - protein, 322 aa, Oryza sativa
MGAPATAASGGGDDDDRDVVFEYLLCTEEDAASAGSFFQQLQGPAPAVSSSPSTTTATAPAAAGSCDDGGEEEEEEVWTVDVVAELIGGEAERSHSPRADYPGRLRSGRPADLAARADSVAWILKVRELYGMLPVTAYLAVSYMDRFLSLHRLPMEDARYIFEHRTIFRMELLVLDALDWRLRSITPFTFMYLFADKVDPNGKHIRELIHQATQVTLATIHDTEFLDHCPSSIAAAAVLCASSEIMQLVSIDHGTLVSWRIIGLDEEAIIRCYRLMQQLISSNNVGRESTEITMATTTTTATTAVSSEEVVSSSPPSKRRKM

SEQ ID NO: 84 - protein, 383 aa, Oryza sativa
MAPSSSSCHDAAASMLLCAEDNSSILWLEDEEGEVGERRSGGCRSMVGDLAAGGGGGSGGGGVEEEEDMFPRQSEECVASLVEREQAHMPRADYGERLRGGGGDVDLRVRSEAIGWIWEVYTYYNFSSVTAYLAVNYLDRFLSQYELPEGRDWMTQLLSVACLSIAAKMEETVVPQCLDLQIGEPRFLFEVETIHRMELLVLTNLNWRMQAVTPFSYIDYFLRELNSGNAAPRSWLLRSSELILRIAAGTGFLEFRPSEIAAAVAATVAGEATGVVEEDIAEAFTHVDKGRVLQCQEAIQDHHYSMATINTVQPKPASTRRGSASASSSSVPESPVAVLDAGCLSYKSDDTDAATIASHGGGRRKSCFDSSPVTSKKRRKLSR

SEQ ID NO: 85 - protein, 356 aa, Oryza sativa
MGVLCFGASNILLCAEDSSSVLGLGGFGGGGGEVAAELGCGGGGGFDFFGFGGGAVFPIDSDEFVALLVEKEMDHQPQRGYLEKLELGGLECSWRKDAIDWICKVHSYYNFGPLSLYLAVNYLDRFLSSFNLPHDESWMQQLLSVSCLSLATKMEETVVPLPMDLQVFDAEYVFEARHIKRMELIVMKTLKWRLQAVTPFSFIGYFLDKFNEGKPPSYTLASWCSDLTVGTLKDSRFLSFRPSEIAAAVVLAVLAENQFLVFNSALGESEIPVNKEMVMRCYELMVEKALVKKIRNSNASSSVPHSPITVLDAACFSFRSDDTTLGSSQSNSNNKDYNSQDSAPASKRRRLNTTPI

SEQ ID NO: 86 - protein, 342 aa, Oryza sativa
MAPSFDFAASILLCAEDNTAILDLGEESEEISWVVGVDASLGDLSMDFPLQSDDCIEALLGREEQQHIPMEGYLQRLLLQPDGLDLVAVRSDAIDWIWKVHELYKFGPLTAVLSVNYLDRFLSVFDLPQEEACMTQLLAVASLSLAAKMEETVVPHPLDLQVCDAKYVFETRTIKRMELAVLNALKWRMQAVTACSFIDYYLHKFNDDDTPSTSALSRSVDLILSTCKVAEFLVFRPSEIAASVALVALEEHETSMFERVATCYKNLKKERVLRCYEMIQDKIIMRNIMRQSAGSVFSIPKSPIGVLDAAACISQQSEDTFVGSPATNYESSASSKRRRICR

SEQ ID NO: 87 - protein, 356 aa, Oryza sativa
MAALTSYEMAASILLCAEDSSSVLGFGGEEEEEEEDVVAGKRARCAGPPPPPCVDVAGVDFAVPSEECVARLVETEADHMPREDYAERLRAGGGDGDLDLRVRMDAIDWIWKVHSYYSFAPLTACLAVNYLDRFLSLYQLPDGKDWMTQLLAVACLSLAAKMEETDVPQSLDLQVGEERYVFEAKTIQRMELLVLSTLKWRMQAVTPFSYVDYFLRELNGGDPPSGRSALLSSELILCIARGTECLGFRPSEIAAAVAAAVVGEEHAAFSHVNKERMSHCQEVIQAMELIHPKPASPSRVFVSSSIPRSPTGVLDAAGCLSYRSDDSAVASHYAASSWGYEHDSSPVSSKRRKISR

SEQ ID NO: 88 - protein, 365 aa, Oryza sativa
MSMEEAEECSAACGFSLTCQEDGADLGDGVVDDDDDGDVFLFYNAVAAADDEEEEEEYVEQMVSKEASFCCSSSSSLFDAAAGDGYGDGDGDGDWFRQARLAAIKWILETRGYFGFGHRTAYLAIAYFDRFCLRRRVDREAMPWAARLLSIACVSVAAKMEEYQSPALSEFDAGGGRVFCSDSIRRMELLVLSTLGWRMGAVTPFDFLPCFSSRLHRHHHGGAGAAGHGAAAAARVALNAVGFTFATAEAGSVLDYRPSTVAAAAILAASYGAPLTKEALESKMSNLSPSCLIDKENVHACYSMMVGDMNNNRRSSKRPLQCSDSNEITTTSTYDSVLVDDVTDTAAFAATAMNKRLRPEPPRIR

SEQ ID NO: 89 - protein, 367 aa, Oryza sativa
MEAEDEYSAGCSFSLMCQEDSTDLDDDGGGGGCFAGDGRADLLLVYNAAAAADDEDEEEVEEYMDHLVSKESSFCSSSSSTSSSSCCFSDAGGESAAAAAPMDWFALARRATVKWILETRGCFGFCHRTAYLAIAYFDRFCLRRCIDRSVMPWAARLLAVACVSLAAKMEEYRAPALSEFRAGVGDDGYEFSCVCIRRMELLVLSTLDWRMAAVTPFDYLPCLSSRLRRHVGGGGGAGASAALIFSAAEAASVLDHRPSTVAAAAVLAATHGALTREALESKMSGLSPSFLLDKEDVFACYSAMLSQPTSPASKSTTTTTGKRSSSSSCSESTDAASSYDATAASFPAAASCGSKRMRLELPGGILR

SEQ ID NO: 90 - protein, 345 aa, Oryza sativa
MGDASASTSAPATPTSTLICREDGNDLFSADPADDDGGGGSGGDWELSIADDDHVLLMDRDDEYLALMLSKERCAGGGGGGERGDEEEEEMVEEWMKNARSWCVGWIVKTNAGFRFSLKTAYVAVSYLDRFLARRCVDRDKEWALQLLSVACLSLAAKVEERRPPRLPEFKLDMYDCASLMRMELLVLTTLKWQMITETPFSYLNCFTAKFRHDERKAIVLRAIECIFASIKVISSVGYQPSTIALAAILIARNKETAPNLDELKSVVGSLWQQLDTGHVYSCYNKMMIQEDRSMQSTTEVASSGVSVAHIGGSEDSAMGGANNATTLEATPDKKRKRLHSPQRQ

SEQ ID NO: 91 - protein, 320 aa, Oryza sativa
MDMATGAKEVVVVEAYEYEFDLENPFTSPADEPIASLLDAEGHHSPSVSAAASAARREAAGFISKVRYDGELDVHPRVAYLALNYVDRYLSKRQLACERNPWAPRLLAISCLTLAAKMQRAAAISAADIQRGEEFMFDEAKIQRMEQMVLNALEWRTRSVTPLAFLGFFLSACFPQPRHPALLDAIKARAVDLLLRVQPEVKMAEFSPSVAAAAALLAAAGEVAGAHLLGFEAGVAACPFVNSEKLRECGEVMAAACGVGPSWAAAATSAETPVTVLGHHRSASSESERTTTVGSAANSADAKRRCMGPPRQWGVGGPDE

SEQ ID NO: 92 - protein, 359 aa, Saccharum officinarum
  misc_feature (188)..(189): Xaa can be any naturally occurring amino acid
MTPRFDFAASVLLCSEDSTTIFDLEEEEREGILCVLRPSSRHASAPSGALSIDFPLQSDSCIEAFLGREEDHLPMEGYAERLLLQQPGGSDLVAIRNYAIDWIWKVHDYYKLGPLTVVLSVNYMDRFLSVYHNALEKDWMTQLLTVACLSLAVKMEETIVFNPLNLQVVDAEYVFEPSTIHRMEVLVXXKHRMEVLVLHTXSWRMQAVTPCSFIDYYLHQFSDGDVVSEIILSRTVELILXAHLKLLSFWFSKPSEIAASIALVALGKRDSSVLESVATCRKELRKERVLGCYEMVQDKIVTGDIVIKSDGSSVFPKQHSPTGVLAVVACESQQSEDTSAGATVCNESSSARKRRRICR

SEQ ID NO: 93 - protein, 353 aa, Triticum aestivum
MVPSGYDCAASVLLCAEDNAAILGLDDDEEDCSWAAAAATPPRIAADAAAAAEGFLVDHPVQSDECVAALVETEKEHMPADGYPQMLLRRPGALDLAAVRRDAIDWIWEVIEHFNFAPLTAVLSVNYLDRFLSVYPLPEGKAWVTQLLAVACLSLASKMEETYVPLPVDLQVVEANSAFEGRTIKRMELLVLSTLKWRMQAVTACSFIDYFLRKFNDHDAPSMLAFSRSTDLILSTAKGADFLVFRPSEIAASVALAAFGERNTSVVERATTTCKFINKERVLRCYELIQDKVAMGTIVLKSAGSSMFSVPQSPIGVSDAAACLSQQSDDTAVGSPATCYQASSASKRRRIGR

SEQ ID NO: 94 - protein, 358 aa, Zea mays
MVPGYDCAASVLLCAEDNAAILGLDDDGEESSWAAAATPPRDTVAAAAATGVAVDGILTEFPLLSDDCVATLVEKEVEHMPAEGYLQKLQRRHGDLDLAAVRKDAIDWIWKVIEHYNFAPLTAVLSVNYLDRFLSTYEFPEGRAWMTQLLAVACLSLASKIEETFVPLPLDLQVAEAKFVFEGRTIKRMELLVLSTLKWRMHAVTACSFVEYFLHKLSDHGAPSLLARSRSSDLVLSTAKGAEFVVFRPSEIAASVALAAIGECRSSVIERAASSCKYLDKERVLRCHEMIQEKITAGSIVLKSAGSSISSVPQSPIGVLDAAACLSQQSDDATVGSPAVCYHSSSTSKRRRITRRLL

(19) United States
(12) Patent Application Publication
(10) Pub. No.: US 2008/0163394 A1
(43) Pub. Date: Jul. 3, 2008

(54) Plants Having Increased Yield And Method For Making The Same

(75) Inventors: Valerie Frankard, Waterloo (BE); Vladimir Mironov, Gent (BE)

Correspondence Address: CONNOLLY BOVE LODGE & HUTZ, LLP, P O BOX 2207, WILMINGTON, DE, 19899, US

(73) Assignee: CropDesign N.V., Zwijnaarde (BE)

(21) Appl. No.: 11/887,056

(22) PCT Filed: Mar. 24, 2006
(86) PCT No.: PCT/EP2006/002895

§ 371 Date: Nov. 12, 2007

Related U.S. Application Data
(60) Provisional application No. 60/668,076, filed on Apr. 5, 2005.

(30) Foreign Application Priority Data
Mar. 25, 2005 (EP) 05102444.6

(51) Int. Cl. C12N 15/54; A01H 5/00

(52) U.S. Cl. 800/287; 800/298; 536/23.2

(57) ABSTRACT

The present invention concerns a method for increasing plant yield relative to corresponding wild type plants. More specifically, the present invention concerns a method for increasing plant yield comprising introducing into a plant a nucleic acid encoding a cyclin D3 (CYCD3) polypeptide under the control of a promoter capable of preferentially expressing the nucleic acid in the endosperm of seeds. The present invention also concerns plants comprising an isolated nucleic acid encoding a CYCD3 polypeptide under the control of a promoter capable of preferentially expressing the nucleic acid in the endosperm of seeds, which plants have increased yield relative to corresponding wild type plants. The invention also provides constructs useful in the methods of the invention.

The present invention relates generally to the field of molecular biology and concerns a method for increasing plant yield relative to corresponding wild type plants. More specifically, the present invention concerns a method for increasing plant yield comprising introducing into a plant a nucleic acid encoding a cyclin D3 (CYCD3) polypeptide under the control of a promoter capable of preferentially expressing the nucleic acid in the endosperm of seeds. The present invention also concerns plants comprising an isolated nucleic acid encoding a CYCD3 polypeptide under the control of a promoter capable of preferentially expressing the nucleic acid in the endosperm of seeds, which plants have increased yield relative to corresponding wild type plants. The invention also provides constructs useful in the methods of the invention.

The ever-increasing world population and the dwindling supply of arable land available for agriculture fuels research towards improving the efficiency of agriculture. Conventional means for crop and horticultural improvements utilise selective breeding techniques to identify plants having desirable characteristics. However, such selective breeding techniques have several drawbacks, namely that these techniques are typically labour intensive and result in plants that often contain heterogeneous genetic components that may not always result in the desirable trait being passed on from parent plants. Advances in molecular biology have allowed mankind to modify the germplasm of animals and plants. Genetic engineering of plants entails the isolation and manipulation of genetic material (typically in the form of DNA or RNA) and the subsequent introduction of that genetic material into a plant. Such technology has the capacity to deliver crops or plants having various improved economic, agronomic or horticultural traits.

A trait of particular economic interest is yield. Yield is normally defined as the measurable produce of economic value from a crop and may be defined in terms of quantity and/or quality. Yield is directly dependent on several factors, for example, the number and size of the organs, plant architecture (for example, the number of branches), seed production and more. Root development, nutrient uptake and stress tolerance may also be important factors in determining yield. Optimizing one of the abovementioned factors may therefore contribute to increasing crop yield.

A trait of particular economic interest is seed yield. Plant seeds are an important source of human and animal nutrition. Crops such as, corn, rice, wheat, canola and soybean account for over half of the total human caloric intake, whether through direct consumption of the seeds themselves or through consumption of meat products raised on processed seeds.

They are also a source of sugars, oils and many kinds of metabolites used in industrial processes. Seeds contain an embryo, the source of new shoots and roots after germination, and an endosperm, the source of nutrients for embryo growth, during germination and early growth of seedlings. The development of a seed involves many genes, and requires the transfer of metabolites from roots, leaves and stems into the growing seed. The endosperm, in particular, assimilates the metabolic precursors of carbohydrate polymers, oil and proteins and synthesizes them into storage macromolecules to fill out the grain.

The ability to increase plant yield, whether through altering seed-related traits, such as seed number, seed biomass, seed development, seed filling or any other seed-related trait, or whether by increasing the number and size of plant organs, or by influencing plant architecture (for example, the number of branches), root development, nutrient uptake or stress tolerance, would have many applications in agriculture, and even many non-agricultural uses, such as in the biotechnological production of substances such as pharmaceuticals, antibodies or vaccines.

One of the ways in which plant yield may be increased is by altering the inherent growth mechanisms of a plant. The inherent growth mechanisms of a plant reside in a highly ordered sequence of events collectively known as the ‘cell cycle’. Progression through the cell cycle is fundamental to the growth and development of all multicellular organisms and is crucial to cell proliferation. The major components of the cell cycle are highly conserved in yeast, mammals, and plants. The cell cycle is typically divided into the following sequential phases: G0-G1-S-G2-M. DNA replication or synthesis generally takes place during the S phase (“S” is for DNA synthesis) and mitotic segregation of the chromosomes occurs during the M phase (the “M” is for mitosis), with intervening gap phases, G1 (during which cells grow before DNA replication) and G2 (a period after DNA replication during which the cell prepares for division). Cell division is completed after cytokinesis, the last step of the M phase. Cells that have exited the cell cycle and that have become quiescent are said to be in the G0 phase. Cells in this phase can be stimulated to renter the cell cycle at the G1 phase. The “G” in G1, G2 and G0 stands for “gap”. Completion of the cell cycle process allows each daughter cell during cell division to receive a full copy of the parental genome.

Cell division is controlled by two principal cell cycle events, namely initiation of DNA synthesis and initiation of mitosis. Each transition to each of these key events is controlled by a checkpoint represented by specific protein complexes (involved in DNA replication and division). The expression of genes necessary for DNA synthesis at the G1/S boundary is regulated by the E2F family of transcription factors in mammals and plant cells (La Thangue, 1994; Muller et al., 2001; De Veylder et al. 2002). Entry into the cell cycle is regulated/triggered by an E2F/Rb complex that integrates signals and allows activation of transcription of cell cycle genes. The transition between the different phases of the cell cycle, and therefore progression through the cell cycle, is driven by the formation and activation of different heterodimeric serine/threonine protein kinases, generally referred to as cyclin-dependent kinases (CDKs). A prerequisite for activity of these kinases is the physical association with a specific cyclin, the timing of activation being largely dependent upon cyclin expression. Cyclin binding induces conformational changes in the N-terminal lobe of the associating CDK and contributes to the localisation and substrate specificity of the complex. Monomeric CDKs are activated when they are associated with cyclins and thus have a kinase activity. Cyclin protein levels fluctuate in the cell cycle and therefore represent a major factor in determining timing of CDK activation. The periodic activation of these complexes containing cyclins and CDK during cell cycle mediates the temporal regulation of cell-cycle transitions (checkpoints).

Cyclins can be grouped into mitotic cyclins (designated A- and B-type cyclins in higher eukaryotes and CLBs in budding yeast) and G1-specific cyclins (designated D-type cyclins in mammals and CLNs in budding yeast). H-type cyclins regulate the activity of the CAKs (CDK-activating kinases). All four types of cyclins known in plants were identified mostly by analogy to their human counterparts. In Arabidopsis, ten A-type, nine B-type, ten D-type and one H-type cyclin have been described (Vandepoele et al., 2002).

The ten D-type cyclins in Arabidopsis are subdivided into seven subclasses, D1 to D7, which reflect their lack of high sequence similarity to each other, which is in contrast to the A-type and B-type cyclins. Only the D3 and D4 subclasses have more than one member, respectively three and two. Redundancy of the D3-type cyclins has been proposed previously as an explanation for the failure to observe mutant phenotypes upon knocking out of a single D3-type cyclin (Swaminathan et al., 2000). The two D3-type cyclins are linked via a recent segmental duplication, which suggests that these are functionally redundant. A similar hypothesis could hold for D4-type cyclins, because two out of three are located in a duplicated block.

The much larger divergence seen for D-type cyclins compared with A- and B-type cyclins might reflect the presumed role of D-type cyclins in integrating developmental signals and environmental cues into the cell cycle. For example, D3-type cyclins have been shown to respond to plant hormones, such as cytokinins and brassinosteroids, whereas CYCD2 and CYCD4 are activated earlier in G1 and react to sugar availability (for review, see Stals and Inzé, 2001).

Overexpression of the CYCD2;1 gene in tobacco was reported to increase cell division and increase overall plant growth rate with no morphological alterations (Cockcroft et al., 2000).

Overexpression in Arabidopsis of the CYCD3;1 gene under the control of a CaMV 35S promoter was reported to give plants with enlarged cotyledons, a dramatically reduced final plant size and distorted development. At a cellular level, cells are pushed from G1, causing ectopic cell divisions in both meristematic regions and in regions in which cell division is normally absent or limited. This increase in cell numbers is coupled to a decrease in cell size (Dewitte et al., 2003).

It is an object of the present invention to overcome some of the problems associated with the prior art expression of CYCD3 in plants.

It has now been found that introducing into a plant a nucleic acid encoding a CYCD3 polypeptide under the control of a promoter capable of preferentially expressing the nucleic acid in the endosperm of seeds gives plants having increased yield relative to corresponding wild type plants in particular relative to transgenic plants under the control of promoters which are not capable of preferably driving expression in the endosperm. Therefore according to one embodiment of the present invention, there is provided a method for increasing plant yield, comprising introducing into a plant a nucleic acid encoding a CYCD3 polypeptide under the control of a promoter capable of preferentially expressing the nucleic acid in the endosperm of seeds.

Advantageously, performance of the methods according to the present invention results in plants having increased yield, particularly seed yield, relative to corresponding wild type plants.

The term “increased yield” as defined herein is taken to mean an increase in any one or more of the following, each relative to corresponding wild type plants: (i) increased biomass (weight) of one or more parts of a plant, which may include aboveground (harvestable) parts and/or (harvestable) parts below ground, especially increased root biomass; (ii) increased total seed yield, which includes an increase in seed biomass (seed weight) and which may be an increase in the seed weight per plant and/or on an individual seed basis and/or per hectare or acre; (iii) increased number of flowers (“florets”) per panicle (iv) increased number of (filled) seeds; (v) increased seed size, which may also influence the composition of seeds; (vi) increased seed volume, which may also influence the composition of seeds (including oil, protein and carbohydrate total content and composition); (vii) increased individual seed area and/or seed perimeter; (viii) increased individual seed length and/or width; (ix) increased harvest index, which is expressed as a ratio of the yield of harvestable parts, such as seeds, over the total biomass; and (x) increased thousand kernel weight (TCKW), which is extrapolated from the number of filled seeds counted and their total weight. An increased TKW may result from an increased seed size and/or seed weight. An increased TKW may result from an increase in embryo size (weight) and/or endosperm size (weight). An increase in seed size, seed volume, seed area and seed length may be due to an increase in specific parts of a seed, for example due to an increase in the size of the embryo and/or endosperm and/or aleurone and/or scutellum and/or cotyledons, or other parts of a seed.

Taking corn as an example, a yield increase may be manifested as one or more of the following: increase in the number of plants per hectare or acre, an increase in the number of ears per plant, an increase in the number of rows, number of kernels per row, kernel weight, thousand kernel weight, ear length/diameter, increase in the seed filling rate (which is the number of filled seeds divided by the total number of seeds and multiplied by 100), among others. Taking rice as an example, a yield increase may be manifested by an increase in one or more of the following: number of plants per hectare or acre, number of panicles per plant, number of spikelets per panicle, number of flowers per panicle, increase in the seed filling rate, increase in TKW, among others. An increase in yield may also result in modified architecture, or may occur as a result of modified architecture.

According to a preferred feature, performance of the methods of the invention result in plants having increased seed yield. In particular, such increased seed yield includes increased number of flowers per panicle, increased total seed yield, increased TKW and increased harvest index, each relative to corresponding wild type plants. Therefore, according to the present invention, there is provided a method for increasing seed yield in plants, which method comprises introducing into a plant a nucleic acid encoding a CYCD3 polypeptide under the control of a promoter capable of preferentially expressing the nucleic acid in the endosperm of seeds.

Since the transgenic plants according to the present invention have increased yield, it is likely that these plants exhibit an increased growth rate (during at least part of their life cycle), relative to the growth rate of corresponding wild type plants at a corresponding stage in their life cycle. The increased growth rate may be specific to one or more parts of a plant (including seeds), or may be throughout substantially the whole plant. A plant having an increased growth rate may even exhibit early flowering. The increase in growth rate may take place at one or more stages in the life cycle of a plant or during substantially the whole plant life cycle.

Increased growth rate during the early stages in the life cycle of a plant may reflect enhanced vigour. The increase in growth rate may alter the harvest cycle of a plant allowing plants to be sown later and/or harvested sooner than would otherwise be possible. If the growth rate is sufficiently increased, it may allow for the further sowing of seeds of the same plant species (for example sowing and harvesting of rice plants followed by sowing and harvesting of further rice plants all within one conventional growing period). Similarly, if the growth rate is sufficiently increased, it may allow for the further sowing of seeds of different plants species (for example the sowing and harvesting of rice plants followed by, for example, the sowing and optional harvesting of soybean, potato or any other suitable plant). Harvesting additional times from the same rootstock in the case of some crop plants may also be possible. Altering the harvest cycle of a plant may lead to an increase in annual biomass production per acre (due to an increase in the number of times (say in a year) that any particular plant may be grown and harvested). An increase in growth rate may also allow for the cultivation of transgenic plants in a wider geographical area than their wild-type counterparts, since the territorial limitations for growing a crop are often determined by adverse environmental conditions either at the time of planting (early season) or at the time of harvesting (late season). Such adverse conditions may be avoided if the harvest cycle is shortened. The growth rate may be determined by deriving various parameters from growth curves, such parameters may be: T-Mid (the time taken for plants to reach 50% of their maximal size) and T-90 (time taken for plants to reach 90% of their maximal size), amongst others.

Performance of the methods of the invention gives plants having an increased growth rate. Therefore, according to the present invention, there is provided a method for increasing plant growth rate relative to the growth rate of corresponding wild type plants, which method comprises introducing into a plant a nucleic acid encoding a CYCD3 polypeptide under the control of a promoter capable of preferentially expressing the nucleic acid in the endosperm of seeds.

An increase in yield and/or growth rate occurs whether the plant is under non-stress conditions or whether the plant is exposed to various stresses compared to control plants. Plants typically respond to exposure to stress by growing more slowly. In conditions of severe stress, the plant may even stop growing altogether. Mild stress on the other hand is defined herein as being any stress to which a plant is exposed which does not result in the plant ceasing to grow altogether without the capacity to resume growth. Due to advances in agricultural practices (irrigation, fertilization, pesticide treatments) severe stresses are not often encountered in cultivated crop plants. As a consequence, the compromised growth induced by mild stress is often an undesirable feature for agriculture. Mild stresses are the typical stresses to which a plant may be exposed. These stresses may be the everyday biotic and/or abiotic (environmental) stresses to which a plant is exposed. Typical abiotic or environmental stresses include temperature stresses caused by atypical hot or cold/freezing temperatures; salt stress; water stress (drought or excess water). Chemicals may also cause abiotic stresses. Biotic stresses are typically those stresses caused by pathogens, such as bacteria, viruses, fungi and insects.

Advantageously, performance of the methods of the invention allows yield to be increased in any plant.

The term ‘plant’ as used herein encompasses whole plants, ancestors and progeny of the plants and plant parts, including seeds, shoots, stems, leaves, roots (including tubers), flowers, and tissues and organs, wherein each of the aforementioned comprise the gene/nucleic acid of interest. The term “plant” also encompasses plant cells, suspension cultures, callus tissue, embryos, meristematic regions, gametophytes, sporophytes, pollen and microspores, again wherein each of the aforementioned comprise the gene/nucleic acid of interest.

Plants that are particularly useful in the methods of the invention include all plants which belong to the superfamily Viridiplantae, in particular monocotyledonous and dicotyledonous plants including fodder or forage legumes, ornamental plants, food crops, trees or shrubs selected from the list comprising Acacia spp., Acer spp., Actinidia spp., Aesculus spp., Agathis australis, Albizia amara, Alsophila tricolor, Andropogon spp., Arachis spp, Areca catechu, Astella fragrans, Astragalus cicer, Baikiaea plurijuga, Betula spp., Brassica spp., Bruguiera gymnorrhiza, Burkea africana, Butea frondosa, Cadaba farinosa, Calliandra spp, Camellia sinensis, Canna indica, Capsicum spp., Cassia spp., Centroema pubescens, Chaenomeles spp., Cinnamomum cassia, Coffea arabica, Colophospermum mopane, Coronillia varia, Cotoneaster serotina, Crataegus spp., Cucumis spp., Cupressus spp., Cyathea dealbata, Cydonia oblonga, Cryptomeria japonica, Cymbopogon spp., Cynthea dealbata, Cydonia oblonga, Dalbergia monetaria, Davallia divaricata, Desmodium spp., Dicksonia squarosa, Diheteropogon amplectens, Dioclea spp, Dolichos spp., Dorycnlum rectum, Echinochloa pyramidalis, Ehrartia spp., Eleusine coracana, Eragrestis spp., Erythrina spp., Eucalyptus spp., Euclea schimperi, Eulalla villosa, Fagopyrum spp., Feijoa sellowiana, Fragaria spp., Flemingia spp, Freycinetia banksi, Geranium thunbergii, Ginkgo biloba, Glycine javanica, Gliricidia spp, Gossypium hirsutum, Grevillea spp., Gulbourtia coleosperma, Hedysarum spp., Hemarthia altissima, Heteropogon contortus, Hordeum vulgare, Hyparrhenia rufa, Hypericum erectum, Hyperthelia dissoluta, Indigo incamata, Iris spp., Leptarrhena pyrollfolia, Lespediza spp., Lettuca spp., Leucaena leucocephala, Loudetia simplex, Lotonus bainesii, Lotus spp., Macrotyloma axillare, Malus spp., Manihot esculenta, Medicago sativa, Metasequoia glyptostroboides, Musa sapientum, Nicotianum spp., Onobrychis spp., Omithopus spp., Oryza spp., Peltophorum africanum, Pennisetum spp., Persea gratissima, Petunia spp., Phaseolus spp., Phoenix canariensis, Phormium cookianum, Photinia spp., Picea glauca, Pinus spp., Pisum sativum, Podocarpus totara, Pogonarthria fleckii, Pogonarthria squarrosa, Populus spp., Prosopis cineraria, Pseudotsuga menziesii, Pterolobium stellatum, Pyrus communis, Quercus spp., Rhaphiolepsis umbellata, Rhopalostylis sapida, Rhus natalensis, Ribes grossularia, Ribes spp., Robinia pseudoacacia, Rosa spp., Rubus spp., Salix spp., Schyzachyrium sanguineum, Sciadopitys verticillata, Sequoia sempervirens, Sequoiadendron giganteum, Sorghum bicolor, Spinacia spp., Sporobolus fimbriatus, Stiburus alopecuroides, Stylosanthos humilis, Tadehagi spp, Taxodium distichum, Themeda triandra, Trifolium spp., Triticum spp., Tsuga heterophylla, Vaccinium spp., Vicia spp., Vitis vinifera, Watsonia pyramidata, Zantedeschia aethiopica, Zea mays, amaranth, artichoke, asparagus, broccoli, Brussels sprouts, cabbage, canola, carrot, cauliflower, celery, collard greens, flax, kale, lentil, oilseed rape, okra, onion, potato, rice, soybean, strawberry, sugarbeet, sugarcane, sunflower, tomato, squash, tea and algae, amongst others.

According to a preferred embodiment of the present invention, the plant is a crop plant. Examples of such crop plants include soybean, sunflower, canola, alfalfa, rapeseed, cotton, tomato, potato and tobacco, amongst others. Further preferably, the plant is a monocotyledonous plant. One such example of a monocotyledonous plant is sugarcane. More preferably the plant is a cereal. Examples of such cereals include rice, maize, wheat, barley, millet, rye, sorghum and oats.

A CYCD3 polypeptide may be identified using different methods. For example, the query protein sequence may be BLASTed (for example, using BLAST default parameters for the gap opening penalty and the gap extension penalty) against a translated Arabidopsis nucleic acid sequence database. The first hit from the BLAST result will be an Arabidopsis CYCD3 polypeptide. Another method for identifying a CYCD3 polypeptide is by aligning the query sequence with known CYCD3 protein sequences, using for example the AlignX program from Vector NTI suite (InforMax, Bethesda, Md.). Multiple alignments may then be carried out with a gap opening penalty of 10 and a gap extension of 0.01. Minor manual editing of the alignment may also be necessary in order to better position some conserved regions. If the query sequence is a CYCD3 polypeptide, it will align with the known CYCD3 polypeptide sequences.

“CYCD3 polypeptide” as defined herein refers to any polypeptide sequence which, when used in the construction of a cyclin or cyclin D phylogenetic tree, such as the one depicted in FIG. 1, falls into the cyclin D3-type group which includes CYCD3 polypeptides (and not other D-type cyclins, such as cyclin D1, D2, D4, D5, D6 and D7). Performance of the methods of the invention requires the use of nucleic acids encoding CYCD3 polypeptides. Reference herein to a nucleic acid encoding a CYCD3 polypeptide is to a nucleic acid encoding a CYCD3 polypeptide as defined above.

A person skilled in the art could readily determine whether any polypeptide sequence in question falls within the aforementioned definition using known techniques and software for the making of such a phylogenetic tree, such as a GCG, EBI or CLUSTAL package, using default parameters. Upon construction of such a phylogenetic tree, sequences clustering in the D3-type cyclin group will be considered to fall within the definition of a “CYCD3 polypeptide”. Nucleic acids encoding such sequences will be useful in performing the methods of the invention.

D3-type cyclins typically have the ability to bind and activate plant CDKs and Rb. In addition to a cyclin box and an LxCxE motif within the first 40 or so amino acids (which is characteristic of most D-type cyclins), D3-type cyclins may comprise one or more and preferably all of the conserved regions identified by the boxes shown in FIGS. 2 and 6; As shown in FIGS. 2 and 6, one mismatch within the boxes is allowed.

Examples of nucleic acids encoding CYCD3 polypeptides falling under the aforementioned definition of a CYCD3 polypeptide are given in Table 1 below. The CYCD3-encoding nucleic acids shown in Table 1 may be useful in performing the methods of the invention, i.e. to obtain plants having improved yield relative to corresponding wild type plants by introducing and expressing any one of these nucleic acids under the control of a promoter capable of preferentially expressing the nucleic acids in the endosperm of seeds. Variants of the CYCD3-encoding nucleic acids of Table 1 are also advantageously useful in the methods of the invention. SEQ ID NO: 1, SEQ ID NO: 48 or variants of either are preferred for use in the methods of the present invention.

Variants of a nucleic acid encoding a CYCD3 polypeptide as defined herein typically encode a substantial portion of the complete protein which may comprise in addition to a cyclin box and an LxCxE motif within the first 40 or so amino acids (which is characteristic of most D-type cyclins), one or more and preferably all of the conserved regions identified by the boxes shown in FIGS. 2 and 6 (as shown in FIGS. 2 and 6, one mismatch within the boxes is allowed).

Examples of CYCD3 polypeptides as defined hereinabove are shown in Table 1 (encoded by polynucleotide sequences with NCBI accession number). Preferred CYCD3 polypeptide sequence for the performance of the invention is represented by SEQ ID NO: 2, SEQ ID NO: 49 or a substantial portion of either.

The CYCD3 polypeptides may be the complete protein encoded by the nucleic acids, or may be portions of the encoded protein. Preferably, the nucleic acids provided herein encode CYCD3 polypeptides constituting a substantial portion of the complete protein which comprises, in addition to a cyclin box and an LxCxE motif within the first 40 or so amino acids (which is characteristic of most D-type cyclins), one or more and preferably all of the conserved regions identified by the boxes shown in FIGS. 2 and 6 (as shown in FIGS. 2 and 6, one mismatch within the boxes is allowed). The portion may be used in isolated form or it may be fused to other coding (or non coding) sequences in order to, for example, produce a protein that combines several activities. When fused to other coding sequences, the resulting polypeptide produced upon translation may be bigger than that predicted for the CYCD3 fragment.

TABLE 1
Examples of nucleic acids encoding CYCD3 polypeptides

| | NCBI nucleic | | | |
|---|---|---|---|---|
| | acid accession | | SEQ ID NO of | SEQ ID NO of |
| Name | number | Source | nucleic acid | polypeptide |
| Antma_cycD3a | AJ250397 | Antirrhinum majus | SEQ ID NO: 6 | SEQ ID NO: 7 |
| Antma_cycD3b | AJ250398 | Antirrhinum majus | SEQ ID NO: 8 | SEQ ID NO: 9 |
| Arath_CYCD3; 1 | NM_119579.2 | Arabidopsis thaliana | SEQ ID NO: 10 | SEQ ID NO: 11 |
| Arath_CYCD3; 2 | NM_126126.2 | Arabidopsis thaliana | SEQ ID NO: 12 | SEQ ID NO: 13 |
| Arath_CYCD3; 3 | NM_114867.2 | Arabidopsis thaliana | SEQ ID NO: 1 | SEQ ID NO: 2 |
| Eupes_cycD3; 2 | AY340588 | Euphorbia esula | SEQ ID NO: 14 | SEQ ID NO: 15 |
| Eupes_cycD3; 1 | AY340589 | Euphorbia esula | SEQ ID NO: 16 | SEQ ID NO: 17 |
| Helan_cycD3 | AY033440 | Helianthus annuus | SEQ ID NO: 18 | SEQ ID NO: 19 |
| Heltu_cycD3; 1 | AY063461 | Helianthus tuberosus | SEQ ID NO: 20 | SEQ ID NO: 21 |
| Lagsi_cycD3; 1 | AF519810 | Lagenaria siceraria | SEQ ID NO: 22 | SEQ ID NO: 23 |
| Lagsi_cycD3; 2 | AF519811 | Lagenaria siceraria | SEQ ID NO: 24 | SEQ ID NO: 25 |
| Lyces_cycD3; 1 | AJ002588 | Lycopersicum | SEQ ID NO: 26 | SEQ ID NO: 27 |
| | | esculentum | | |
| Lyces_cycD3; 2 | AJ002589 | Lycopersicum | SEQ ID NO: 28 | SEQ ID NO: 29 |
| | | esculentum | | |
| Lyces_cycD3; 3 | AJ002590 | Lycopersicum | SEQ ID NO: 30 | SEQ ID NO: 31 |
| | | esculentum | | |
| Medsa_cycD3 | X88864 | Medicago sativa | SEQ ID NO: 32 | SEQ ID NO: 33 |
| Nicta_cycD3; 1 | AJ011893 | Nicotiana tabacum | SEQ ID NO: 34 | SEQ ID NO: 35 |
| Nicta_cycD3; 2 | AJ011894 | Nicotiana tabacum | SEQ ID NO: 36 | SEQ ID NO: 37 |
| Nicta_cycD3; 3 | AB015222 | Nicotiana tabacum | SEQ ID NO: 38 | SEQ ID NO: 39 |
| Orysa_cycD3-like | AK103499.1 | Oryza sativa | SEQ ID NO: 40 | SEQ ID NO: 41 |
| Pissa_cycD3 | AB008188 | Pisum sativum | SEQ ID NO: 42 | SEQ ID NO: 43 |
| Popal_cycD3 | AY230139 | Populus alba | SEQ ID NO: 44 | SEQ ID NO: 45 |
| Poptr_cycD3 | AF181993 | Populus tremula x | SEQ ID NO: 46 | SEQ ID NO: 47 |
| | | Populus tremuloides | | |
| *Arath_cycD3_modified | NA | Arabidopsis thaliana | SEQ ID NO: 48 | SEQ ID NO: 49 |
| Aqufo_CycD3 | DT755971.1 | Aquilegia formosa x | SEQ ID NO: 50 | SEQ ID NO: 51 |
| | DT749271 | Aquilegia pubescens | | |
| Camsi_CycD3 | AB247282 | Camellia sinensis | SEQ ID NO: 52 | SEQ ID NO: 53 |
| Camsi_CycD3; 2 | AB247283 | Camellia sinensis | SEQ ID NO: 54 | SEQ ID NO: 55 |
| Citsi_CycD3 | CX676162 | Citrus sinensis | SEQ ID NO: 56 | SEQ ID NO: 57 |
| | CX676163 | | | |
| Glyma_CycD3 | AY439098 | Glycine max | SEQ ID NO: 58 | SEQ ID NO: 59 |
| Goshi_CycD3 | DT571998 | Gossypium hirsutum | SEQ ID NO: 60 | SEQ ID NO: 61 |
| | DT543827.1 | | | |
| Lotco_CycD3 | AP008090 | Lotus corniculatus | SEQ ID NO: 62 | SEQ ID NO: 63 |
| Medtr_CycD3 | DY615448.1 | Medicago trunculata | SEQ ID NO: 64 | SEQ ID NO: 65 |
| Scuba_CycD3 | AB205135.1 | Scutellaria baicalensis | SEQ ID NO: 66 | SEQ ID NO: 67 |
| Zeama_CycD3 like 2 | DV509394.1 | Zea mays | SEQ ID NO: 68 | SEQ ID NO: 69 |
| | DV028752.1 | | | |
| Zeama_CycD3 like 3 | DT948601.1 | Zea mays | SEQ ID NO: 70 | SEQ ID NO: 71 |
| | DT642394.1 | | | |

*Contains no stop codon, which generates a longer transcript; the resultant extra portion is not believed to affect overall function compared to a corresponding non-modified sequence (SEQ ID NO: 2).

Also useful in the methods of in the present invention are variants of the CYCD3-encoding nucleic acids provided herein. Such variants may be derived from any natural or artificial source. The nucleic acid/gene or variant thereof may be isolated from a microbial source, such as yeast or fungi, or from a plant, algae or animal (including human) source. This nucleic acid may be modified from its native form in composition and/or genomic environment through deliberate human manipulation. The nucleic acid is preferably of plant origin, whether from the same plant species (for example to the one in which it is to be introduced) or whether from a different plant species. The nucleic acid may be isolated from a dicotyledonous species, preferably from the family Brassicaceae, further preferably from Arabidopsis thaliana. More preferably, the CYCD3-encoding nucleic acid isolated from Arabidopsis thaliana is represented by SEQ ID NO: 1 or SEQ ID NO: 48, and the CYCD3 polypeptide sequence is as represented by SEQ ID NO: 2 or SEQ ID NO: 49.

An example of a variant of a CYCD3-encoding nucleic acid/gene is a nucleic acid capable of hybridising under reduced stringency conditions, preferably under stringent conditions, with a CYCD3-encoding nucleic acid/gene encoding a polypeptide which, when used in the construction of a cyclin or cyclin D phylogenetic tree falls into a cyclin D3-type group which includes the CYCD3 as in SEQ ID NO: 2 or SEQ ID NO: 49. Preferably, a variant of a CYCD3-encoding nucleic acid/gene is a nucleic acid capable of hybridising to a nucleic acid encoding a CYCD3 polypeptide, which polypeptide comprises, in addition to a cyclin box and an LxCxE motif within the first 40 or so amino acids, one or more and preferably all of the conserved regions identified by the boxes shown in FIGS. 2 and 6 (as shown in FIGS. 2 and 6, one mismatch within the boxes is allowed). Preferred is a nucleic acid capable of hybridising to a nucleic acid represented by SEQ ID NO: 1 or SEQ ID NO: 48. Also useful in the methods of the invention is any nucleic acid capable of hybridising to any of the CYCD3-encoding nucleic acids shown in Table 1.

The term “hybridisation” as defined herein is a process wherein substantially homologous complementary nucleotide sequences anneal to each other. The hybridisation process can occur entirely in solution, i.e. both complementary nucleic acids are in solution. The hybridisation process can also occur with one of the complementary nucleic acids immobilised to a matrix such as magnetic beads, Sepharose beads or any other resin. The hybridisation process can furthermore occur with one of the complementary nucleic acids immobilised to a solid support such as a nitro-cellulose or nylon membrane or immobilised by e.g. photolithography to, for example, a siliceous glass support (the latter known as nucleic acid arrays or microarrays or as nucleic acid chips). In order to allow hybridisation to occur, the nucleic acid molecules are generally thermally or chemically denatured to melt a double strand into two single strands and/or to remove hairpins or other secondary structures from single stranded nucleic acids. The stringency of hybridisation is influenced by conditions such as temperature, salt concentration, ionic strength and hybridisation buffer composition.

“Stringent hybridisation conditions” and “stringent hybridisation wash conditions” In the context of nucleic acid hybridisation experiments such as Southern and Northern hybridisations are sequence dependent and are different under different environmental parameters. A person skilled in the art will be aware of various parameters which may be altered during hybridisation and washing and which will either maintain or change the stringency conditions.

The Tₘ is the temperature under defined ionic strength and pH, at which 50% of the target sequence hybridises to a perfectly matched probe. The Tₘ is dependent upon the solution conditions and the base composition and length of the probe. For example, longer sequences hybridise specifically at higher temperatures. The maximum rate of hybridisation is obtained from about 16° C. up to 32° C. below Tₘ. The presence of monovalent cations in the hybridisation solution reduce the electrostatic repulsion between the two nucleic acid strands thereby promoting hybrid formation; this effect is visible for sodium concentrations of up to 0.4M. Formamide reduces the melting temperature of DNA-DNA and DNA-RNA duplexes with 0.6 to 0.7° C. for each percent formamide, and addition of 50% formamide allows hybridisation to be performed at 30 to 45° C., though the rate of hybridisation will be lowered. Base pair mismatches reduce the hybridisation rate and the thermal stability of the duplexes. On average and for large probes, the Tₘ decreases about 1° C. per % base mismatch. The Tₘ may be calculated using the following equations, depending on the types of hybrids:

1. DNA-DNA hybrids (Meinkoth and Wahl, Anal. Biochem., 138: 267-284, 1984):

Tₘ=81.5° C.+16.6×log [Na⁺]^{a}+0.41×%[G/C^{b}]−500×[L^{c}]⁻¹−0.61×% formamide

2. DNA-RNA or RNA-RNA hybrids:

Tₘ=79.8+18.5(log₁₀ [Na⁺]^{a})+0.58(%G/C^{b})+11.8(%G/C^{b})²−820/L^{c}

3. oligo-DNA or oligo-RNA^{d} hybrids:

For <20 nucleotides: Tₘ=2(Iₙ)

For 20-35 nucleotides: Tₘ=22+1.46(Iₙ)

^{a}or for other monovalent cation, but only accurate in the 0.01-0.4 M range.^{b}only accurate for % GC in the 30% to 75% range.^{c}L=length of duplex in base pairs.^{d}Oligo, oligonucleotide; Iₙ, effective length of primer=2×(no. of G/C)+(no. of A/T).

Note: for each 1% formamide, the Tₘ is reduced by about 0.6 to 0.7° C., while the presence of 6 M urea reduces the Tₘ by about 30° C.

Specificity of hybridisation is typically the function of post-hybridisation washes. To remove background resulting from non-specific hybridisation, samples are washed with dilute salt solutions. Critical factors of such washes include the Ionic strength and temperature of the final wash solution: the lower the salt concentration and the higher the wash temperature, the higher the stringency of the wash. Wash conditions are typically performed at or below hybridisation stringency. Generally, suitable stringent conditions for nucleic acid hybridisation assays or gene amplification detection procedures are as set forth above. Conditions of greater or less stringency may also be selected. Generally, low stringency conditions are selected to be about 50° C. lower than the thermal melting point (Tₘ) for the specific sequence at a defined ionic strength and pH. Medium stringency conditions are when the temperature is 20° C. below Tₘ, and high stringency conditions are when the temperature is 10° C. below Tₘ. For example, stringent conditions are those that are at least as stringent as, for example, conditions A-L; and reduced stringency conditions are at least as stringent as, for example, conditions M-R. Non-specific binding may be controlled using any one of a number of known techniques such as, for example, blocking the membrane with protein containing solutions, additions of heterologous RNA, DNA, and SDS to the hybridisation buffer, and treatment with RNase. Examples of hybridisation and wash conditions are listed in Table 2 below.

TABLE 2
Examples of hybridisation and wash conditions

| | | | | Wash |
|---|---|---|---|---|
| Stringency | Polynucleotide | Hybrid | Hybridization Temperature | Temperature |
| Condition | Hybrid^{±} | Length (bp)^{‡} | and Buffer^{†} | and Buffer^{†} |
| A | DNA:DNA | >or | 65° C. 1xSSC; or 42° C., | 65° C.; |
| | | equal to 50 | 1xSSC and 50% formamide | 0.3xSSC |
| B | DNA:DNA | <50 | Tb*; 1xSSC | Tb*; 1xSSC |
| C | DNA:RNA | >or | 67° C. 1xSSC; or 45° C., | 67° C.; |
| | | equal to 50 | 1xSSC and 50% formamide | 0.3xSSC |
| D | DNA:RNA | <50 | Td*; 1xSSC | Td*; 1xSSC |
| E | RNA:RNA | >or | 70° C. 1xSSC; or 50° C., | 70° C.; |
| | | equal to 50 | 1xSSC and 50% formamide | 0.3xSSC |
| F | RNA:RNA | <50 | Tf*; 1xSSC | Tf*; 1xSSC |
| G | DNA:DNA | >or | 65° C. 4xSSC; or 45° C., | 65° C.; |
| | | equal to 50 | 4xSSC and 50% formamide | 1xSSC |
| H | DNA:DNA | <50 | Th*; 4xSSC | Th*; 4xSSC |
| I | DNA:RNA | >or | 67° C. 4xSSC; or 45° C., | 67° C.; |
| | | equal to 50 | 4xSSC and 50% formamide | 1xSSC |
| J | DNA:RNA | <50 | Tj*; 4xSSC | Tj*; 4xSSC |
| K | RNA:RNA | >or | 70° C. 4xSSC; or 40° C., | 67° C.; |
| | | equal to 50 | 6xSSC and 50% formamide | 1xSSC |
| L | RNA:RNA | <50 | Tl*; 2xSSC | Tl*; 2xSSC |
| M | DNA:DNA | >or | 50° C. 4xSSC; or 40° C., | 50° C.; |
| | | equal to 50 | 6xSSC and 50% formamide | 2xSSC |
| N | DNA:DNA | <50 | Tn*; 6xSSC | Tn*; 6xSSC |
| O | DNA:RNA | >or | 55° C. 4xSSC; or 42° C., | 55° C.; |
| | | equal to 50 | 6xSSC and 50% formamide | 2xSSC |
| P | DNA:RNA | <50 | Tp*; 6xSSC | Tp*; 6xSSC |
| Q | RNA:RNA | >or | 60° C. 4xSSC; or 45° C., | 60° C.; |
| | | equal to 50 | 6xSSC and 50% formamide | 2xSSC |
| R | RNA:RNA | <50 | Tr*; 4xSSC | Tr*; 4xSSC |

^{‡}The “hybrid length” is the anticipated length for the hybridising nucleic acid. When nucleic acids of known sequence are hybridised, the hybrid length may be determined by aligning the sequences and identifying the conserved regions described herein.
^{†}SSPE (1xSSPE is 0.15M NaCl, 10 mM NaH₂PO₄, and 1.25 mM EDTA, pH 7.4) may be substituted for SSC (1xSSC is 0.15M NaCl and 15 mM sodium citrate) in the hybridisation and wash buffers; washes are performed for 15 minutes after hybridisation is complete. The hybridisations and washes may additionally include 5 × Denhardt's reagent, 0.5-1.0% SDS, 100 μg/ml denatured, fragmented salmon sperm DNA, 0.5% sodium pyrophosphate, and up to 50% formamide.
*Tb-Tr: The hybridisation temperature for hybrids anticipated to be less than 50 base pairs in length should be 5-10° C. less than the melting temperature Tₘ of the hybrids; the Tₘ is determined according to the above-mentioned equations.
^{±}The present invention also encompasses the substitution of any one, or more DNA or RNA hybrid partners with either a PNA, or a modified nucleic acid.

For the purposes of defining the level of stringency, reference can be made to Sambrook et al. (2001) Molecular Cloning: a laboratory manual, 3^{rd} Edition Cold Spring Harbor Laboratory Press, CSH, New York or to Current Protocols in Molecular Biology, John Wiley & Sons, N.Y. (1989).

Nucleic acids encoding “homologues” of a CYCD3 polypeptide may also be useful in the present invention. Homologues encompass peptides, oligopeptides, polypeptides, proteins and enzymes having amino acid substitutions, deletions and/or insertions relative to the unmodified protein in question and having similar biological and functional activity as the unmodified protein from which they are derived. To produce such homologues, amino acids of the protein may be replaced by other amino acids having similar properties (such as similar hydrophobicity, hydrophilicity, antigenicity, propensity to form or break α-helical structures or β-sheet structures). Conservative substitution tables are well known in the art (see for example Creighton (1984) Proteins. W.H. Freeman and Company and Table 3 below).

Also encompassed by the term “homologues” are two special forms of homology, which include orthologous sequences and paralogous sequences, which encompass evolutionary concepts used to describe ancestral relationships of genes. The term “paralogous” relates to gene-duplications within the genome of a species leading to paralogous genes. The term “orthologous” relates to homologous genes in different organisms due to speciation. Examples of homologues of a CYCD3 polypeptide are given in Table 1 hereinabove.

Orthologues in, for example, monocot plant species may easily be found by performing a so-called reciprocal blast search. This may be done by a first blast involving blasting a query sequence (for example SEQ ID NO: 1, SEQ ID NO: 2, SEQ ID NO: 48 or SEQ ID NO: 49) against any sequence database, such as the publicly available NCBI database which may be found at http://www.ncbi.nlm.nih.gov. BLASTn or TBLASTX may be used when starting from nucleotide sequence, or BLASTP or TBLASTN when starting from the protein, with standard default values. The BLAST results may optionally be filtered. The full-length sequences of either the filtered results or the non-filtered results are then BLASTed back (second BLAST) against the sequences of the organism from which the query sequence is derived. The results of the first and second BLASTs are then compared. A paralogue is identified if a high-ranking hit from the second blast is from the same species as from which the query sequence is derived; an orthologue is identified if a high-ranking hit is not from the same species as from which the query sequence is derived. High-ranking hits are those having a low E-value. The lower the E-value, the more significant the score (or in other words the lower the chance that the hit was found by chance). Computation of the E-value is well known in the art. In the case of large families, ClustalW may be used, followed by a neighbour joining tree, to help visualize clustering of related genes and to identify orthologues and paralogues.

Orthologues and paralogues identified as described hereinabove are useful in performing the methods of the invention. According to the invention, there is provided a method for increasing plant yield, comprising introducing into a plant a nucleic acid encoding an orthologue or a paralogue of a CYCD3 polypeptide represented by SEQ ID NO: 2 or SEQ ID NO: 49, which nucleic acid is under the control of a promoter capable of preferentially expressing the nucleic acid in the endosperm of seeds.

A homologue may be in the form of a “substitutional variant” of a protein, i.e. where at least one residue in an amino acid sequence has been removed and a different residue inserted in its place. Amino acid substitutions are typically of single residues, but may be clustered depending upon functional constraints placed upon the polypeptide; insertions will usually be of the order of about 1 to 10 amino acid residues. Preferably, amino acid substitutions comprise conservative amino acid substitutions. Conservative substitution tables are readily available in the art. Table 3 below gives examples of conserved amino acid substitutions.

TABLE 3
Examples of conserved amino acid substitutions

| Residue | Conservative Substitutions |
|---|---|
| Ala | Ser |
| Arg | Lys |
| Asn | Gln; His |
| Asp | Glu |
| Gln | Asn |
| Cys | Ser |
| Glu | Asp |
| Gly | Pro |
| His | Asn; Gln |
| Ile | Leu, Val |
| Leu | Ile; Val |
| Lys | Arg; Gln |
| Met | Leu; Ile |
| Phe | Met; Leu; Tyr |
| Ser | Thr; Gly |
| Thr | Ser; Val |
| Trp | Tyr |
| Tyr | Trp; Phe |
| Val | Ile; Leu |

A homologue may also be in the form of an “insertional variant” of a protein, i.e. where one or more amino acid residues are introduced into a predetermined site in a protein. Insertions may comprise N-terminal and/or C-terminal fusions as well as Intra-sequence Insertions of single or multiple amino acids. Generally, Insertions within the polypeptide sequence will be smaller than N- or C-terminal fusions, of the order of about 1 to 10 residues. Examples of N- or C-terminal fusion proteins or peptides include the binding domain or activation domain of a transcriptional activator as used in the yeast two-hybrid system, phage coat proteins, (histidine)-6-tag, glutathione S-transferase-tag, protein A, maltose-binding protein, dihydrofolate reductase, Tag•100 epitope, c-myc epitope, FLAG®-epitope, lacZ, CMP (calmodulin-binding peptide), HA epitope, protein C epitope and VSV epitope.

Homologues in the form of “deletion variants” of a protein are characterised by the removal of one or more amino acids from a protein.

Polypeptide variants of a protein may readily be made using peptide synthetic techniques well known in the art, such as solid phase peptide synthesis and the like, or by recombinant DNA manipulations. Methods for the manipulation of DNA sequences to produce substitution, insertion or deletion variants of a protein are well known in the art. For example, techniques for making substitution mutations at predetermined sites in DNA are well known to those skilled in the art and include M13 mutagenesis, T7-Gen in vitro mutagenesis (USB, Cleveland, Ohio), QuickChange Site Directed mutagenesis (Stratagene, San Diego, Calif.), PCR-mediated site-directed mutagenesis or other site-directed mutagenesis protocols.

The CYCD3 polypeptide may be a derivative. “Derivatives” of a protein encompass peptides, oligopeptides, polypeptides comprising naturally occurring altered (glycosylated, acylated, ubiquinated, prenylated, phosphorylated, myristoylated, sulphated etc) or non-naturally altered amino acid residues compared to the amino acid sequence of a naturally-occurring form of the polypeptide. A derivative may also comprise one or more non-amino acid substituents or additions compared to the amino acid sequence from which it is derived, for example a reporter molecule or other ligand, covalently or non-covalently bound to the amino acid sequence, such as a reporter molecule which is bound to facilitate its detection, and non-naturally occurring amino acid residues relative to the amino acid sequence of a naturally-occurring protein.

The CYCD3 polypeptide may be encoded by an alternative splice variant of a CYCD3-encoding nucleic acid/gene. The term “alternative splice variant” as used herein encompasses variants of a nucleic acid sequence in which selected introns and/or exons have been excised, maintained, replaced or added, or in which introns have been shortened or lengthened. Such variants will be ones in which the biological activity of the protein is retained, which may be achieved by selectively retaining functional segments of the protein. Such splice variants may be found in nature or may be manmade. Methods for making such splice variants are well known in the art.

According to the invention, there is provided a method for increasing plant yield, comprising introducing into a plant a splice variant of a nucleic acid encoding a CYCD3 polypeptide under the control of a promoter capable of preferentially expressing the nucleic acid in the endosperm of seeds.

Preferred splice variants are splice variants of a nucleic acid encoding a polypeptide which, when used in the construction of a cyclin or cyclin D phylogenetic tree, falls into the D3-type group which includes the CYCD3 represented by SEQ ID NO: 2 or SEQ ID NO: 49. Such splice variants may be splice variants of any of the nucleic acids mentioned in Table 1 above. Splice variants of SEQ ID NO: 1 or SEQ ID NO: 48 are particularly preferred for use in the methods of the invention.

The CYCD3 polypeptide may also be encoded by an allelic variant of a CYCD3-encoding nucleic acid/gene. Allelic variants exist in nature, and encompassed within the methods of the present invention is the use of these natural alleles. Allelic variants encompass Single Nucleotide Polymorphisms (SNPs), as well as Small Insertion/Deletion Polymorphisms (INDELs). The size of INDELs is usually less than 100 bp. SNPs and INDELs form the largest set of sequence variants in naturally occurring polymorphic strains of most organisms.

According to the invention, there is provided a method for increasing plant yield, comprising introducing into a plant an allelic variant of a nucleic acid encoding a CYCD3 polypeptide under the control of a promoter capable of preferentially expressing the nucleic acid in the endosperm of seeds.

Preferred allelic variants are allelic variants of a nucleic acid encoding a polypeptide which, when used in the construction of a cyclin or cyclin D phylogenetic tree falls into the D3-type group which includes the CYCD3 as in SEQ ID NO: 2 or SEQ ID NO: 49. Such allelic variants may be allelic variants of any of the nucleic acids mentioned in Table 1 above. Allelic variants of SEQ ID NO: 1 or SEQ ID NO: 48 are particularly preferred for use in the methods of the invention.

Site-directed mutagenesis and directed evolution are examples of technologies that enable the generation of novel CYCD3 variants.

Several methods are available to achieve site-directed mutagenesis, the most common being PCR based methods (current protocols in molecular biology. Wiley Eds. http://www.4ulr.com/products/currentprotocols/index.html).

Directed evolution, also known as gene shuffling, may also be used to generate variants of CYCD3-encoding nucleic acids. This consists of iterations of DNA shuffling followed by appropriate screening and/or selection to generate variants of CYCD3-encoding nucleic acids or portions thereof encoding CYCD3 polypeptides or portions thereof having a modified biological activity (Castle et al., (2004) Science 304(5674): 1151-4; U.S. Pat. Nos. 5,811,238 and 6,395,547).

Therefore, the nucleic acid introduced into a plant may be one obtained through the techniques of site-directed mutagenesis or directed evolution or any other known method for the generation of such variant sequences.

The nucleic acid to be introduced into a plant may be a full-length nucleic acid or may be a variant sequence as hereinbefore defined. According to a preferred aspect of the present invention, increased expression of a CYCD3-encoding nucleic acid is envisaged. Methods for increasing expression of genes or gene products are well documented in the art and include, for example, overexpression driven by appropriate promoters, the use of transcription enhancers or translation enhancers. Isolated nucleic acids which serve as promoter or enhancer elements may be introduced in an appropriate position (typically upstream) of a non-heterologous form of a polynucleotide so as to upregulate expression of a CYCD3-encoding nucleic acid or variant thereof. For example, endogenous promoters may be altered in vivo by mutation, deletion, and/or substitution (see, Kmiec, U.S. Pat. No. 5,565,350; Zarling et al., PCT/US93/03868), or isolated promoters may be introduced into a plant cell in the proper orientation and distance from a gene of the present invention so as to control the expression of the gene. Methods for reducing the expression of genes or gene products are well documented in the art.

If polypeptide expression is desired, it is generally preferable to include a polyadenylation region at the 3′-end of a polynucleotide coding region. The polyadenylation region can be derived from the natural gene, from a variety of other plant genes, or from T-DNA. The 3′ end sequence to be added may be derived from, for example, the nopaline synthase or octopine synthase genes, or alternatively from another plant gene, or less preferably from any other eukaryotic gene.

An intron sequence may also be added to the 5′ untranslated region or the coding sequence of the partial coding sequence to increase the amount of the mature message that accumulates in the cytosol. Inclusion of a spliceable intron in the transcription unit in both plant and animal expression constructs has been shown to increase gene expression at both the mRNA and protein levels up to 1000-fold, Buchman and Berg, Mol. Cell biol. 8:4395-4405 (1988); Callis et al., Genes Dev. 1:1183-1200 (1987). Such intron enhancement of gene expression is typically greatest when placed near the 5′ end of the transcription unit. Use of the maize introns Adh1-S intron 1, 2, and 6, the Bronze-1 intron are known in the art. See generally, The Maize Handbook, Chapter 116, Freeling and Walbot, Eds., Springer, N.Y. (1994).

The invention also provides genetic constructs and vectors to facilitate introduction and/or expression of the nucleotide sequences useful in the methods according to the invention.

Therefore, there is provided a gene construct comprising:
(i) A nucleic acid encoding a CYCD3 polypeptide;
(ii) One or more control sequences capable of preferentially driving expression of the nucleic acid sequence of (I) in the endosperm of seeds; and optionally
(iii) A transcription termination sequence.

The nucleic acid encoding a CYCD3 polypeptide may be any nucleic acid encoding a CYCD3 polypeptide as defined hereinabove. Particularly preferred are the nucleic acids described in Table 1, particularly the nucleic acid represented by SEQ ID NO: 1 or SEQ ID NO: 48. Also preferred are nucleic acid variants of the nucleic acids described in Table 1, such variants being as defined above.

Constructs useful in the methods according to the present invention may be constructed using recombinant DNA technology well known to persons skilled in the art. The gene constructs may be inserted into vectors, which may be commercially available, suitable for transforming into plants and suitable for expression of the gene of interest in the transformed cells. The invention therefore provides use of a gene construct as defined hereinabove in the methods of the invention.

Plants are transformed with a vector comprising the sequence of interest (i.e., a nucleic acid encoding a CYCD3 polypeptide). The sequence of interest is operably linked to one or more control sequences (at least to a promoter capable of preferentially driving expression of the nucleic acid in the endosperm of seeds). The terms “regulatory element”, “control sequence” and “promoter” are all used interchangeably herein and are to be taken in a broad context to refer to regulatory nucleic acid sequences capable of effecting expression of the sequences to which they are ligated. Encompassed by the aforementioned terms are transcriptional regulatory sequences derived from a classical eukaryotic genomic gene (including the TATA box which is required for accurate transcription initiation, with or without a CCAAT box sequence) and additional regulatory elements (i.e. upstream activating sequences, enhancers and silencers) which alter gene expression in response to developmental and/or external stimuli, or in a tissue-specific manner. Also included within the term is a transcriptional regulatory sequence of a classical prokaryotic gene, in which case it may include a −35 box sequence and/or −10 box transcriptional regulatory sequences. The term “regulatory element” also encompasses a synthetic fusion molecule or derivative that confers, activates or enhances expression of a nucleic acid molecule in a cell, tissue or organ. The term “operably linked” as used herein refers to a functional linkage between the promoter sequence and the gene of interest, such that the promoter sequence is able to initiate transcription of the gene of interest.

The promoter capable of preferentially expressing the nucleic acid in the endosperm of seeds is an endosperm-specific promoter. An endosperm-specific promoter refers to any promoter able to preferentially drive expression of the gene of interest in the endosperm. Reference herein to preferentially increasing expression in the endosperm of seeds is taken to mean increasing expression in the endosperm substantially to the exclusion of expression elsewhere in the plant, apart from any residual expression due to leaky promoters. For example, the prolamin promoter shows strong expression in the endosperm, with leakiness in meristem, more specifically the shoot meristem and/or discrimination centre in the meristem.

Preferably, the endosperm-specific promoter is a seed storage protein promoter, more preferably a promoter isolated from a prolamin gene, such as a rice prolamin RP6 (Wen et al., (1993) Plant Physiol 101(3):1115-6) promoter as represented by SEQ ID NO: 3 or a promoter of similar strength and/or a promoter with a similar expression pattern as the rice prolamin promoter. Similar strength and/or similar expression pattern may be analysed, for example, by coupling the promoters to a reporter gene and checking the function of the reporter gene in tissues of the plant. One well-known reporter gene is beta-glucuronidase and the colorimetric GUS stain used to visualize beta-glucuronidase activity in plant tissue. It should be clear that the applicability of the present invention is not restricted to the nucleic acid represented by SEQ ID NO: 1 or SEQ ID NO: 48, nor is the applicability of the invention restricted to expression of a nucleic acid encoding a CYCD3 polypeptide when driven by a prolamin promoter. Examples of other endosperm-specific promoters that may also be used in performing the methods of the invention are shown in Table 4 below.

TABLE 4
Examples of endosperm-specific promoters for use in the present invention

| GENE SOURCE | REFERENCE |
|---|---|
| glutelin (rice) | Takaiwa et al. (1986) Mol Gen Genet 208: 15-22 |
| | Takaiwa et al. (1987) FEBS Letts. 221: 43-47 |
| zein | Matzke et al., (1990) Plant Mol Biol 14(3): 323-32 |
| wheat LMW and HMW glutenin-1 | Colot et al. (1989) Mol Gen Genet 216: 81-90 |
| | Anderson et al. (1989) NAR 17: 461-2 |
| wheat SPA | Albani et al. (1997) Plant Cell 9: 171-184 |
| wheat gliadins | Rafalski et al. (1984) EMBO 3: 1409-15 |
| barley Itr1 promoter | Diaz et al. (1995) Mol Gen Genet 248(5): 592-8 |
| barley B1, C, D, hordein | Cho et al. (1999) Theor Appl Genet 98: 1253-62 |
| | Muller et al. (1993) Plant J 4: 343-55 |
| | Sorenson et al. (1996) Mol Gen Genet 250: 750-60 |
| barley DOF | Mena et al, (1998) Plant J 116(1): 53-62 |
| blz2 | Onate et al. (1999) J Biol Chem 274(14): 9175-82 |
| synthetic promoter | Vicente-Carbajosa et al. (1998) Plant J 13: 629-640 |
| rice prolamin NRP33 | Wu et al, (1998) Plant Cell Physiol 39(8) 885-889 |
| rice globulin Glb-1 | Wu et al. (1998) Plant Cell Physiol 39(8) 885-889 |
| rice globulin REB/OHP-1 | Nakase et al. (1997) Plant Molec Biol 33: 513-522 |
| rice ADP-glucose PP | Russell et al. (1997) Trans Res 6: 157-68 |
| maize ESR gene family | Opsahl-Ferstad et al. (1997) Plant J 12: 235-46 |
| sorgum kafirin | DeRose et al. (1996) Plant Molec Biol 32: 1029-35 |

Optionally, one or more terminator sequences may also be used in the construct introduced into a plant. The term “terminator” encompasses a control sequence which is a DNA sequence at the end of a transcriptional unit which signals 3′ processing and polyadenylation of a primary transcript and termination of transcription. Additional regulatory elements may include transcriptional as well as translational enhancers. Those skilled in the art will be aware of terminator and enhancer sequences that may be suitable for use in performing the invention. Such sequences would be known or may readily be obtained by a person skilled in the art.

The genetic constructs of the invention may further include an origin of replication sequence that is required for maintenance and/or replication in a specific cell type. One example is when a genetic construct is required to be maintained in a bacterial cell as an episomal genetic element (e.g. plasmid or cosmid molecule). Preferred origins of replication include, but are not limited to, the f1-ori and colE1.

The genetic construct may optionally comprise a selectable marker gene. As used herein, the term “selectable marker gene” includes any gene that confers a phenotype on a cell in which it is expressed to facilitate the identification and/or selection of cells that are transfected or transformed with a nucleic acid construct of the invention. Suitable markers may be selected from markers that confer antibiotic or herbicide resistance, that introduce a new metabolic trait or that allow visual selection. Examples of selectable marker genes include genes conferring resistance to antibiotics (such as nptII that phosphorylates neomycin and kanamycin, or hpt, phosphorylating hygromycin), to herbicides (for example bar which provides resistance to Basta; aroA or gox providing resistance against glyphosate), or genes that provide a metabolic trait (such as manA that allows plants to use mannose as sole carbon source). Visual marker genes result in the formation of colour (for example β-glucuronidase, GUS), luminescence (such as luciferase) or fluorescence (Green Fluorescent Protein, GFP, and derivatives thereof).

The present invention also encompasses plants (and parts thereof) obtainable by the methods according to the present invention. The present invention therefore provides plants obtainable by the method according to the present invention, which plants have introduced and expressed therein a CYCD3-encoding nucleic acid under the control of a promoter capable of preferentially expressing the nucleic acid in the endosperm of seeds.

The invention also provides a method for the production of transgenic plants having increased yield, comprising introduction and expression in a plant of a CYCD3-encoding nucleic acid under the control of a promoter capable of preferentially expressing the nucleic acid in the endosperm of seeds.

More specifically, the present invention provides a method for the production of transgenic plants having increased yield, which method comprises:
(i) introducing and expressing in a plant or plant cell a nucleic acid encoding a CYCD3 polypeptide under the control of a promoter capable of preferentially expressing the nucleic acid in the endosperm of seeds; and
(ii) cultivating the plant cell under conditions promoting plant growth and development.

The increases in yield are as defined above.

The nucleic acid may be introduced directly into a plant cell or into the plant itself (including introduction into a tissue, organ or any other part of a plant). According to a preferred feature of the present invention, the nucleic acid is preferably introduced into a plant by transformation.

The term “transformation” as referred to herein encompasses the transfer of an exogenous polynucleotide into a host cell, irrespective of the method used for transfer. Plant tissue capable of subsequent clonal propagation, whether by organogenesis or embryogenesis, may be transformed with a genetic construct of the present invention and a whole plant regenerated from there. The particular tissue chosen will vary depending on the clonal propagation systems available for, and best suited to, the particular species being transformed. Exemplary tissue targets include leaf disks, pollen, embryos, cotyledons, hypocotyls, megagametophytes, callus tissue, existing meristematic tissue (e.g., apical meristem, axillary buds, and root meristems), and induced meristem tissue (e.g., cotyledon meristem and hypocotyl meristem). The polynucleotide may be transiently or stably introduced into a host cell and may be maintained non-integrated, for example, as a plasmid. Alternatively, it may be integrated into the host genome. The resulting transformed plant cell may then be used to regenerate a transformed plant in a manner known to persons skilled in the art.

Transformation of plant species is now a fairly routine technique. Advantageously, any of several transformation methods may be used to introduce the gene of interest into a suitable ancestor cell. Transformation methods include the use of liposomes, electroporation, chemicals that increase free DNA uptake, injection of the DNA directly into the plant, particle gun bombardment, transformation using viruses or pollen and microprojection. Methods may be selected from the calcium/polyethylene glycol method for protoplasts (Krens, F. A. et al., (1982) Nature 296, 72-74; Negrutiu I et al. (1987) Plant Mol Biol 8: 363-373); electroporation of protoplasts (Shillito R. D. et al. (1985) Bio/Technol 3, 1099-1102); microinjection into plant material (Crossway A et al., (1986) Mol. Gen Genet 202: 179-185); DNA or RNA-coated particle bombardment (Klein T M et al., (1987) Nature 327: 70) infection with (non-integrative) viruses and the like. Transgenic rice plants expressing a CYCD3-encoding nucleic acid/gene are preferably produced via Agrobacterium-mediated transformation using any of the well known methods for rice transformation, such as described in any of the following: published European patent application EP 1198985 A1, Aldemita and Hodges (Planta 199: 612-617, 1996); Chan et al. (Plant Mol Biol 22 (3): 491-506, 1993), Hiei et al. (Plant J 6 (2): 271-282, 1994), which disclosures are incorporated by reference herein as if fully set forth. In the case of corn transformation, the preferred method is as described in either Ishida et al. (Nat. Biotechnol 14(6): 745-50, 1996) or Frame et al. (Plant Physiol 129(1): 13-22, 2002), which disclosures are incorporated by reference herein as if fully set forth.

Generally after transformation, plant cells or cell groupings are selected for the presence of one or more markers which are encoded by plant-expressible genes co-transferred with the gene of interest, following which the transformed material is regenerated into a whole plant.

Following DNA transfer and regeneration, putatively transformed plants may be evaluated, for instance using Southern analysis, for the presence of the gene of interest, copy number and/or genomic organisation. Alternatively or additionally, expression levels of the newly introduced DNA may be monitored using Northern and/or Western analysis, both techniques being well known to persons having ordinary skill in the art.

The generated transformed plants may be propagated by a variety of means, such as by clonal propagation or classical breeding techniques. For example, a first generation (or T1) transformed plant may be selfed to give homozygous second-generation (or T2) transformants, and the T2 plants further propagated through classical breeding techniques.

The generated transformed organisms may take a variety of forms. For example, they may be chimeras of transformed cells and non-transformed cells; clonal transformants (e.g., all cells transformed to contain the expression cassette); grafts of transformed and untransformed tissues (e.g., in plants, a transformed rootstock grafted to an untransformed scion).

The present invention clearly extends to any plant cell or plant produced by any of the methods described herein, and to all plant parts and propagules thereof. The present invention extends further to encompass the progeny of a primary transformed or transfected cell, tissue, organ or whole plant that has been produced by any of the aforementioned methods, the only requirement being that progeny exhibit the same genotypic and/or phenotypic characteristic(s) as those produced by the parent in the methods according to the invention. The invention also includes host cells containing an isolated CYCD3-encoding nucleic acid. Preferred host cells according to the invention are plant cells. The invention also extends to harvestable parts of a plant such as, but not limited to seeds, leaves, fruits, flowers, stem cultures, rhizomes, tubers and bulbs. The invention furthermore relates to products directly derived from a harvestable part of such a plant, such as dry pellets or powders, oil, fat and fatty acids, starch or proteins.

The present invention also encompasses use of CYCD3-encoding nucleic acids and use of CYCD3 polypeptides.

One such use relates to increasing yield, especially seed yield. The seed yield is as defined hereinabove and preferably includes one or more of the following: increased number of flowers per panicle, increased total seed yield, increased TKW and increased harvest index, each relative to corresponding wild type plants.

CYCD3-encoding nucleic acids or variants thereof, or CYCD3 polypeptides may find use in breeding programmes in which a DNA marker is identified which may be genetically linked to a CYCD3-encoding gene or variant thereof. The CYCD3-encoding nucleic acids/genes or variants thereof, or CYCD3 polypeptides may be used to define a molecular marker. This DNA or protein marker may then be used in breeding programmes to select plants having increased yield. The CYCD3-encoding gene or variant thereof may, for example, be a nucleic acid as represented by SEQ ID NO: 1 or SEQ ID NO: 48.

Allelic variants of a CYCD3-encoding nucleic acid/gene may also find use in marker-assisted breeding programmes. Such breeding programmes sometimes require introduction of allelic variation by mutagenic treatment of the plants, using for example EMS mutagenesis; alternatively, the programme may start with a collection of allelic variants of so called “natural” origin caused unintentionally. Identification of allelic variants then takes place, for example, by PCR. This is followed by a step for selection of superior allelic variants of the sequence in question and which give increased yield. Selection is typically carried out by monitoring growth performance of plants containing different allelic variants of the sequence in question, for example, different allelic variants of SEQ ID NO: 1 or SEQ ID NO: 48. Growth performance may be monitored in a greenhouse or in the field. Further optional steps include crossing plants, in which the superior allelic variant was identified, with another plant. This could be used, for example, to make a combination of interesting phenotypic features.

A CYCD3-encoding nucleic acid or variant thereof may also be used as probes for genetically and physically mapping the genes that they are a part of, and as markers for traits linked to those genes. Such information may be useful in plant breeding in order to develop lines with desired phenotypes. Such use of CYCD3-encoding nucleic acids or variants thereof requires only a nucleic acid sequence of at least 15 nucleotides in length. The CYCD3-encoding nucleic acids or variants thereof may be used as restriction fragment length polymorphism (RFLP) markers. Southern blots (Sambrook J, Fritsch E F and Maniatis T (1989) Molecular Cloning, A Laboratory Manual) of restriction-digested plant genomic DNA may be probed with the CYCD3-encoding nucleic acids or variants thereof. The resulting banding patterns may then be subjected to genetic analyses using computer programs such as MapMaker (Lander et al. (1987) Genomics 1: 174-181) in order to construct a genetic map. In addition, the nucleic acids may be used to probe Southern blots containing restriction endonuclease-treated genomic DNAs of a set of individuals representing parent and progeny of a defined genetic cross. Segregation of the DNA polymorphisms is noted and used to calculate the position of the CYCD3-encoding nucleic acid or variant thereof in the genetic map previously obtained using this population (Botstein et al. (1980) Am. J. Hum. Genet. 32:314-331).

The production and use of plant gene-derived probes for use in genetic mapping is described in Bernatzky and Tanksley (1986) Plant Mol. Biol. Reporter 4: 37-41. Numerous publications describe genetic mapping of specific cDNA clones using the methodology outlined above or variations thereof. For example, F2 intercross populations, backcross populations, randomly mated populations, near isogenic lines (NIL), and other sets of individuals may be used for mapping. Such methodologies are well known to those skilled in the art.

The nucleic acid probes may also be used for physical mapping (i.e., placement of sequences on physical maps; see Hoheisel et al. In: Non-mammalian Genomic Analysis: A Practical Guide, Academic press 1996, pp. 319-346, and references cited therein).

In another embodiment, the nucleic acid probes may be used in direct fluorescence in situ hybridization (FISH) mapping (Trask (1991) Trends Genet. 7:149-154). Although current methods of FISH mapping favor use of large clones (several kb to several hundred kb; see Laan et al. (1995) Genome Res. 5:13-20), improvements in sensitivity may allow performance of FISH mapping using shorter probes.

A variety of nucleic acid amplification-based methods for genetic and physical mapping may be carried out using the nucleic acids. Examples include allele-specific amplification (Kazazian (1989) J. Lab. Clin. Med 11:95-96), polymorphism of PCR-amplified fragments (CAPS; Sheffield et al. (1993) Genomics 16:325-332), allele-specific ligation (Landegren et al. (1988) Science 241:1077-1080), nucleotide extension reactions (Sokolov (1990) Nucleic Acid Res. 18:3671), Radiation Hybrid Mapping (Walter et al. (1997) Nat. Genet. 7:22-28) and Happy Mapping (Dear and Cook (1989) Nucleic Acid Res. 17:6795-6807). For these methods, the sequence of a nucleic acid is used to design and produce primer pairs for use in the amplification reaction or in primer extension reactions. The design of such primers is well known to those skilled in the art. In methods employing PCR-based genetic mapping, it may be necessary to identify DNA sequence differences between the parents of the mapping cross in the region corresponding to the instant nucleic acid sequence. This, however, is generally not necessary for mapping methods.

Yield increases are obtained in the methods of the invention by introducing into a plant a nucleic acid encoding a CYCD3 polypeptide under the control of a promoter capable of preferentially expressing the nucleic acid in the endosperm of seeds. However, such yield increases may also be obtained by other well known techniques, such as T-DNA activation, TILLING and homologous recombination.

T-DNA activation tagging (Hayashi et al. Science (1992) 1350-1353) involves insertion of T-DNA, usually containing a promoter (may also be a translation enhancer or an intron), in the genomic region of the gene of interest or 10 kb up- or downstream of the coding region of a gene in a configuration such that the promoter directs expression of the targeted gene. Typically, regulation of expression of the targeted gene by its natural promoter is disrupted and the gene falls under the control of the newly introduced promoter. The promoter is typically embedded in a T-DNA. This T-DNA is randomly inserted into the plant genome, for example, through Agrobacterium infection and leads to overexpression of genes near the inserted T-DNA. The resulting transgenic plants show dominant phenotypes due to overexpression of genes close to the introduced promoter. The promoter to be introduced may be any promoter capable of preferentially driving expression in the endosperm of seeds.

The technique of TILLING (Targeted Induced Local Lesions In Genomes) may also be used to reproduce the effects of performing the methods of the invention. TILLING is a mutagenesis technology useful to generate and/or identify, and to eventually isolate a CYCD3-encoding nucleic acid with modified expression and/or activity. TILLING also allows selection of plants carrying such mutant variants. These mutant variants may exhibit modified expression, either in strength or in location or in timing (if the mutations affect the promoter, for example). TILLING combines high-density mutagenesis with high-throughput screening methods. The steps typically followed in TILLING are: (a) EMS mutagenesis (Redei G P and Koncz C (1992) In Methods in Arabidopsis Research, Koncz C, Chua N H, Schell J, eds. Singapore, World Scientific Publishing Co, pp. 16-82; Feldmann et al., (1994) In Meyerowitz E M, Somerville C R, eds, Arabidopsis. Cold Spring Harbor Laboratory Press, Cold Spring Harbor, N.Y., pp 137-172; Lightner J and Caspar T (1998) In J Martinez-Zapater, J Salinas, eds, Methods on Molecular Biology, Vol. 82. Humana Press, Totowa, N.J., pp 91-104); (b) DNA preparation and pooling of individuals; (c) PCR amplification of a region of interest; (d) denaturation and annealing to allow formation of heteroduplexes; (e) DHPLC, where the presence of a heteroduplex in a pool is detected as an extra peak in the chromatogram; (f) identification of the mutant individual; and (g) sequencing of the mutant PCR product. Methods for TILLING are well known in the art (McCallum et al., (2000) Nat Biotechnol 18: 455-457; reviewed by Stemple (2004) Nat Rev Genet 5(2): 145-50). Plants carrying such mutant variants have preferentially increased expression of a CYCD3-encoding gene in the endosperm.

T-DNA activation and TILLING are examples of technologies that enable the generation of genetic modifications (preferably in the locus of a gene encoding a CYCD3 polypeptide) that give preferentially increased expression of a nucleic acid encoding a CYCD3 polypeptide in the endosperm of plants. The locus of a gene is defined herein as a genomic region, which includes the gene of interest and 10 kb up- or downstream of the coding region.

Homologous recombination allows introduction in a genome of a selected nucleic acid at a defined selected position. Homologous recombination is a standard technology used routinely in biological sciences for lower organisms such as yeast or the moss Physcomitrella. Methods for performing homologous recombination in plants have been described not only for model plants (Offringa et al. (1990) EMBO J 9(10): 3077-84) but also for crop plants, for example rice (Terada et al. (2002) Nat Biotech 20(10): 1030-4; Iida and Terada (2004) Curr Opin Biotech 15(2): 132-8). The nucleic acid (which may be a CYCD3-encoding nucleic acid or variant thereof as hereinbefore defined) is targeted to the locus of a CYCD3 gene. The nucleic acid to be targeted may be an improved allele used to replace the endogenous gene or may be introduced in addition to the endogenous gene. The nucleic acid to be targeted is preferably the region controlling the natural expression of a nucleic acid encoding a CYCD3 polypeptide in a plant. An endosperm-specific promoter is introduced into this region, in addition to it, or replacing it partly or substantially all of it.

All the methods according to the present invention result in plants having increased yield, as described hereinbefore. These useful traits may also be combined with other economically advantageous traits, such as further yield-enhancing traits, tolerance to various stresses, traits modifying various architectural features and/or biochemical and/or physiological features.

### DESCRIPTION OF FIGURES

The present invention will now be described with reference to the following figures in which:

FIG. 1 is a multiple polypeptide alignment prepared using ClustalW and default values, followed by average distance tree computation. The CYCD3 polypeptide cluster is shown.

FIG. 2 is an alignment of known plant CYCD protein sequences. The sequences were aligned using AlignX program from Vector NTI suite (InforMax, Bethesda, Md.). Multiple alignment was done with a gap opening penalty of 10 and a gap extension of 0.01. Minor manual editing was also carried out where necessary to better position some conserved regions. The line shown indicates the separation of CYCD3 polypeptides from other D-type cyclins. A number of motifs specific to CYCD3 polypeptides are boxed.

FIG. 3 is a similarity/identity matrix prepared using MatGAT (Matrix Global Alignment Tool) which calculates the similarity and identity between every pair of polypeptide sequences in a given data set without requiring pre-alignment of the data. The program performs a series of pairwise alignments using the Myers and Miller global alignment algorithm (with a gap opening penalty of 12, and a gap extension penalty of 2). It then calculates similarity and identity using, for example, Blosum 60 as scoring matrix, and then places the results in a distance matrix. Sequence similarity is shown in the bottom half of the dividing line and sequence identity is shown in the top half of the dividing line. The sequence of SEQ ID NO: 2 is indicated as number 5 in the matrix. Polypeptide sequences having at least 30% sequence identity to the sequence of SEQ ID NO: 2 encompass CYCD3 polypeptides.

FIG. 4 is a binary vector for expression in Oryza sativa of the Arabidopsis thaliana CycD3;3 gene under the control of the prolamin promoter.

FIG. 5 details examples of sequences useful in performing the methods according to the present invention.

FIG. 6 is an alignment only of plant CYCD3 protein sequences. The sequences were aligned using AlignX program from Vector NTI suite (InforMax, Bethesda, Md.). Multiple alignment was done with a gap opening penalty of 10 and a gap extension of 0.01. Minor manual editing was also carried out where necessary to better position some conserved regions. In addition to the cyclin box (marked as ‘X’ below the consensus sequence (Interpro ref: IPR006670)) and the LxCxE motif within the first 40 or so amino acids, a number of motifs specific to CYCD3 polypeptides are identified.

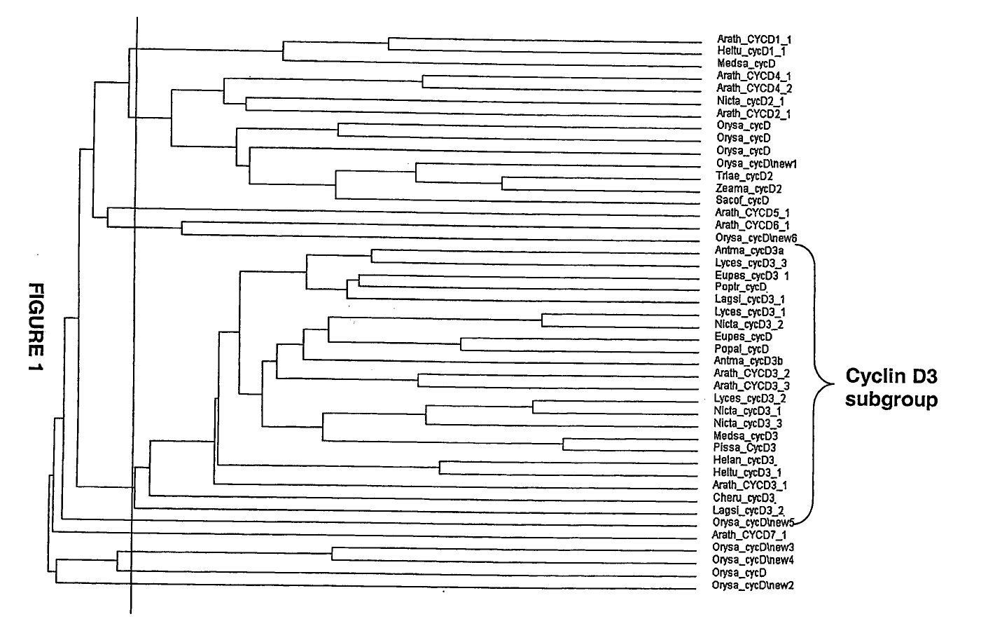

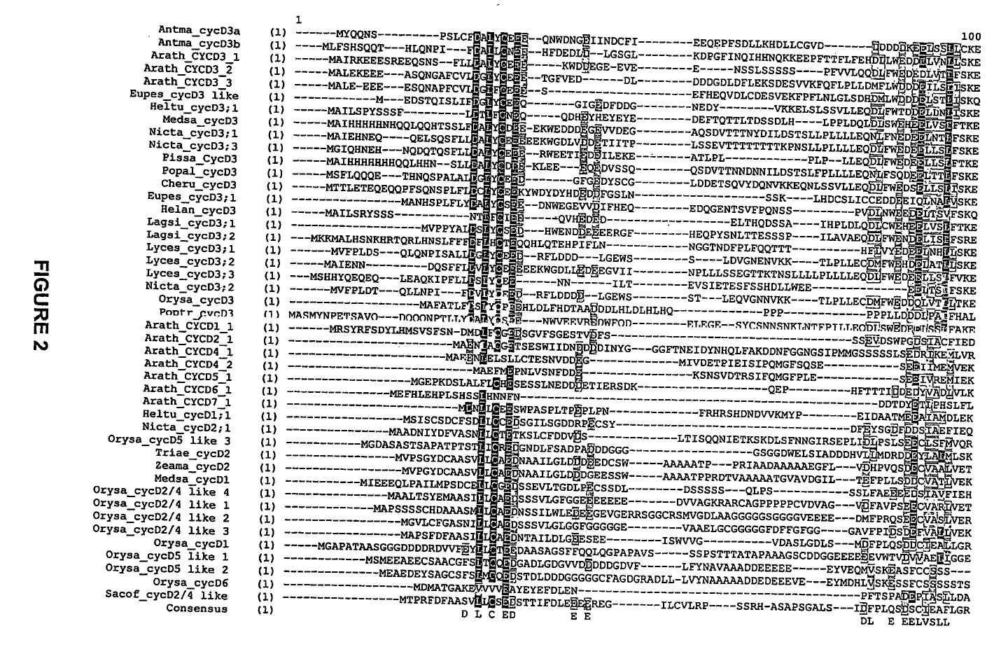

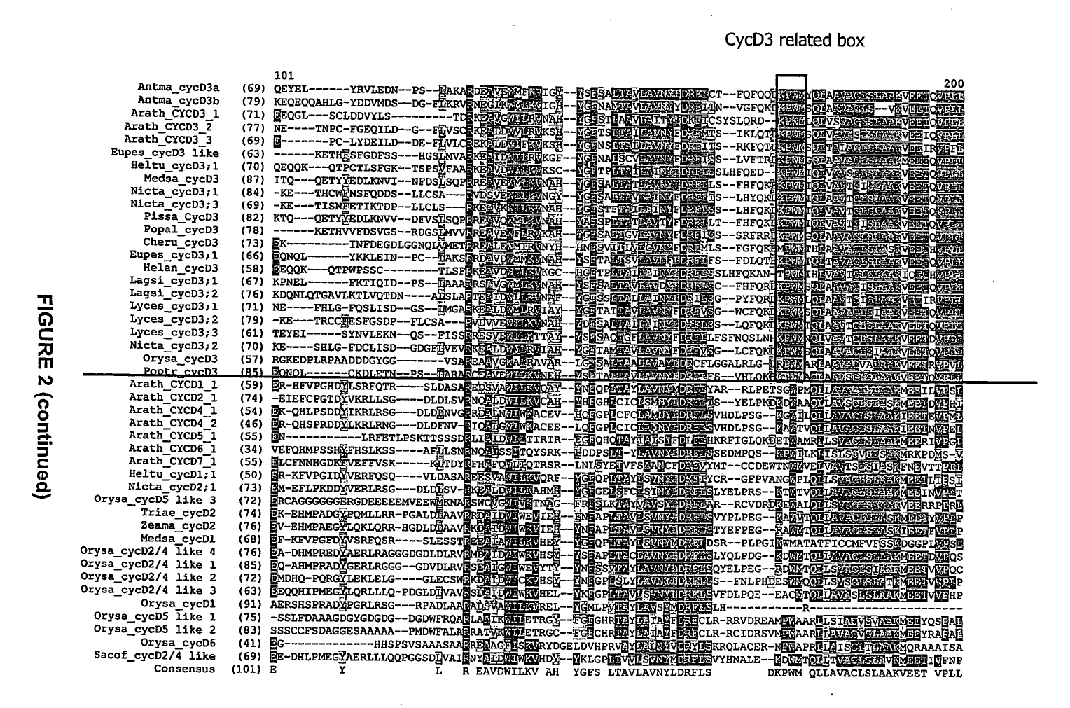

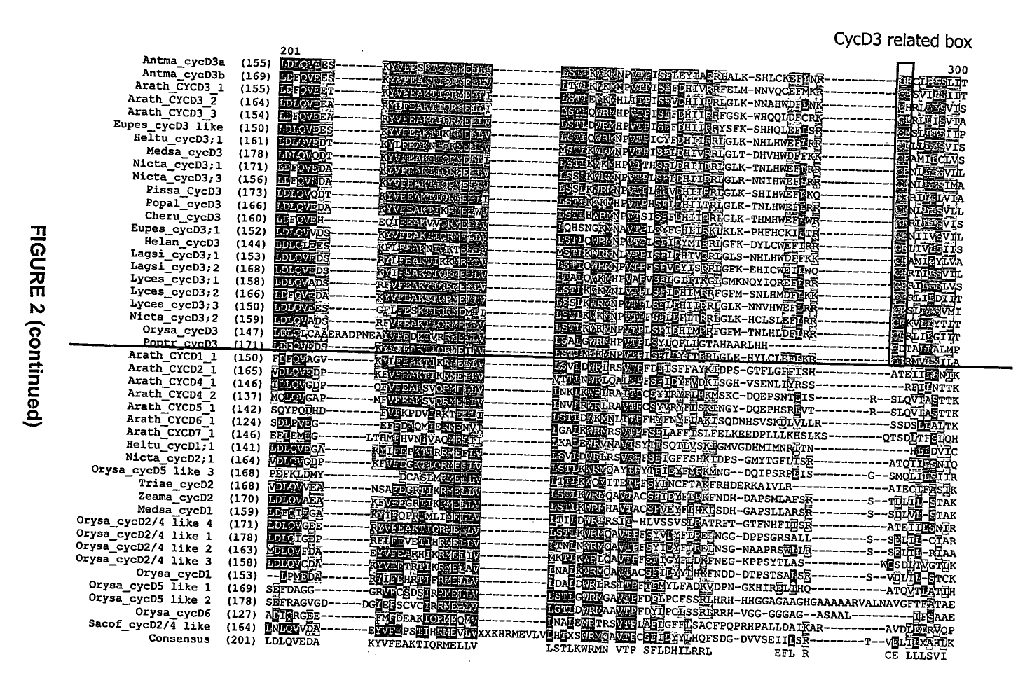

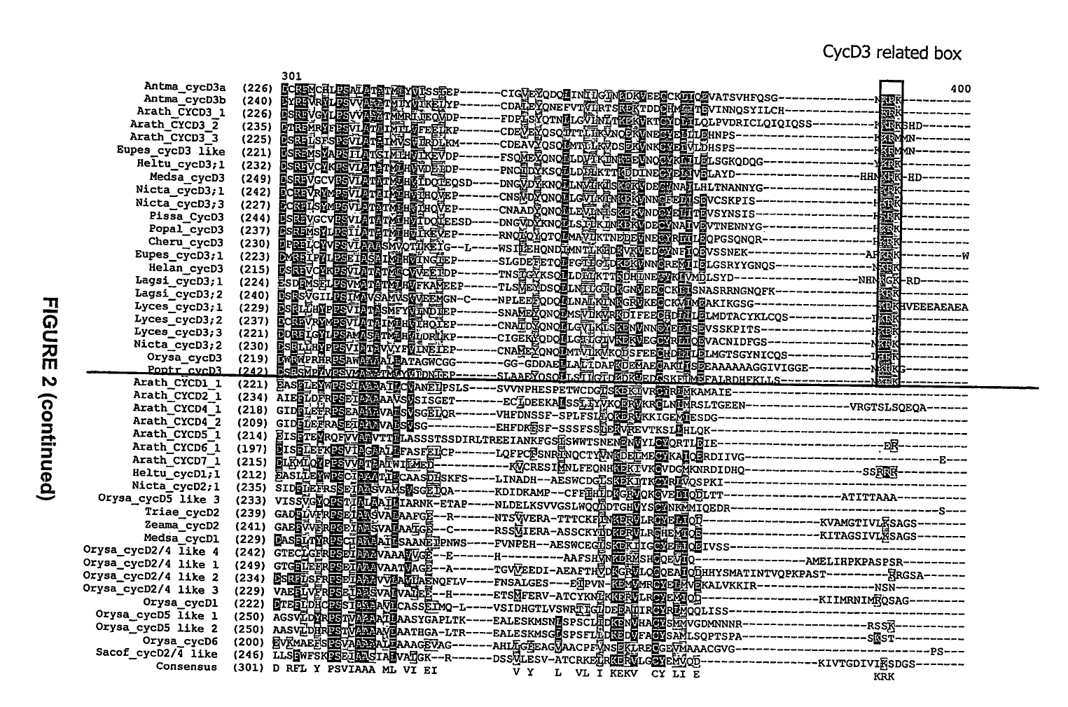

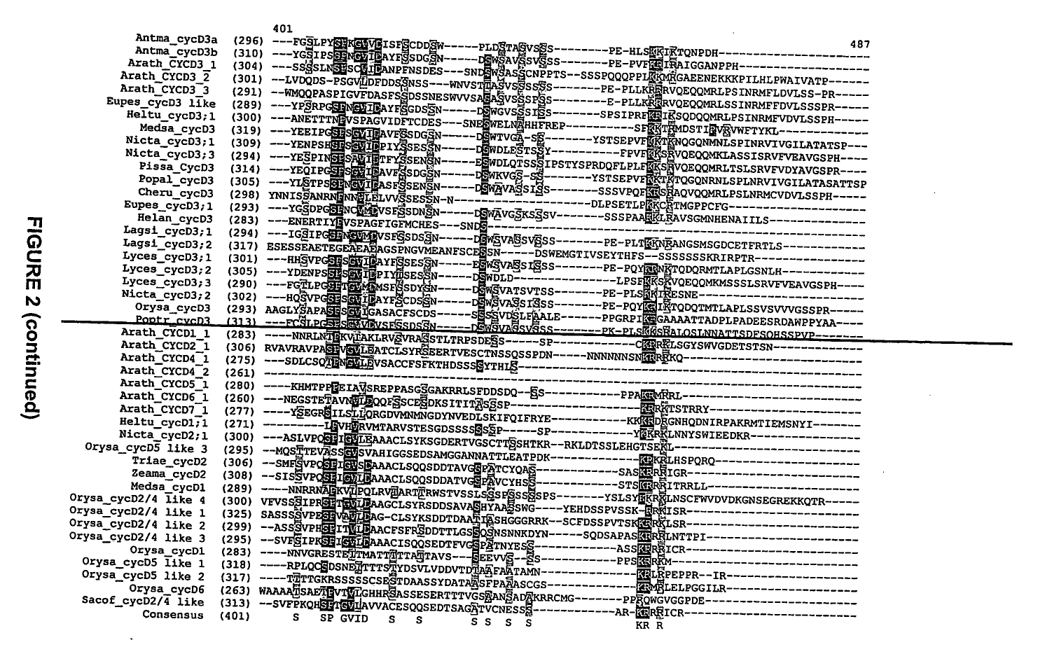

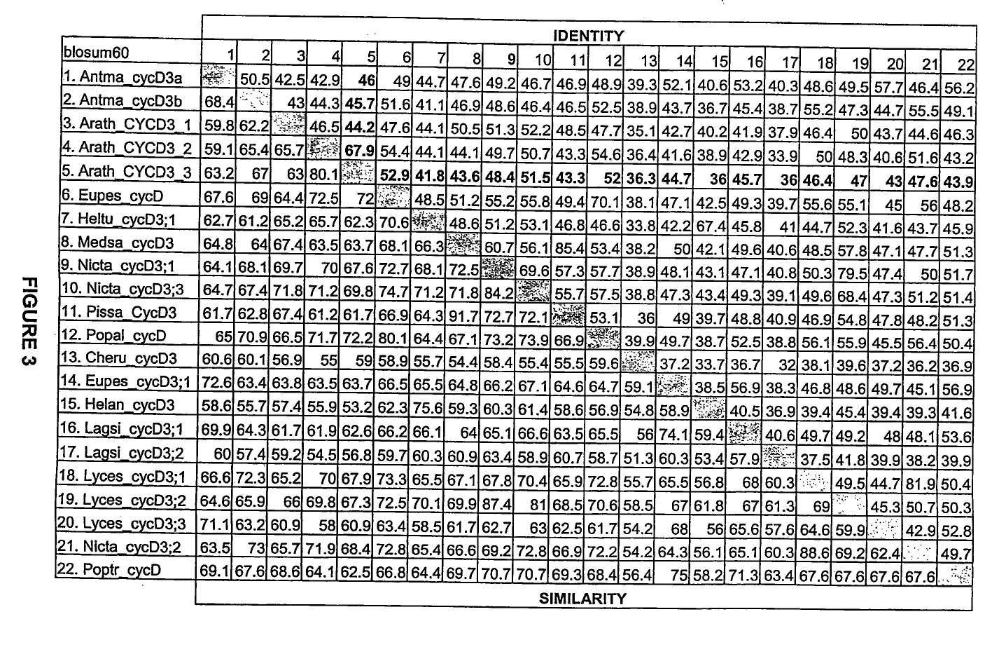

### EXAMPLES

The present invention will now be described with reference to the following examples, which are by way of Illustration alone.

DNA manipulation: unless otherwise stated, recombinant DNA techniques are performed according to standard protocols described in (Sambrook (2001) Molecular Cloning: a laboratory manual, 3rd Edition Cold Spring Harbor Laboratory Press, CSH, New York) or in Volumes 1 and 2 of Ausubel et al. (1994), Current Protocols in Molecular Biology, Current Protocols. Standard materials and methods for plant molecular work are described in Plant Molecular Biology Labfase (1993) by R. D. D. Croy, published by BIOS Scientific Publications Ltd (UK) and Blackwell Scientific Publications (UK).

### Example 1

### Gene Cloning

The Arabidopsis CycD3;3 was amplified by PCR using as template an Arabidopsis thaliana seedling cDNA library (Invitrogen, Paisley, UK). After reverse transcription of RNA extracted from seedlings, the cDNAs were cloned into pCMV Sport 6.0. Average insert size of the bank was 1.5 kb and original number of clones was of 1.59×10⁷ cfu. Original titer was determined to be 9.6×10⁵ cfu/ml after first amplification of 6×10¹¹ cfu/ml. After plasmid extraction, 200 ng of template was used in a 50 μl PCR mix. Primers prm0360 (sense, start codon in bold, AttB1 site in italic: 5′ GGGGACAAGTTTGTACAAAAAAGCAGGCTTCACAATGGCTTTAGAAGAGGAGGA 3′) and prm0361 (reverse, complementary, stop codon in bold, AttB2 site in italic: 5′ GGGGACCACTTTGTACAAGAAAGCTGGGTTTAGCGAGGACTACTACTAAGCA 3′), which include the AttB sites for Gateway recombination, were used for PCR amplification. PCR was performed using Hifi Taq DNA polymerase in standard conditions. A PCR fragment of 1086 bp was amplified and purified also using standard methods. The first step of the Gateway procedure, the BP reaction, was then performed, during which the PCR fragment recombines in vivo with the pDONR201 plasmid to produce, according to the Gateway terminology, an “entry clone”, p0443. Plasmid pDONR201 was purchased from Invitrogen, as part of the Gateway® technology.

For the modified sequence of SEQ ID NO: 48/49, the reverse primer is: 5′ GGGGACCACTTTGTACAAGAAAGCTGGGTTTAGCGAGGACTACTATAAGCA 3′).

### Example 2

### Vector Construction

The entry clone p0443 was subsequently used in an LR reaction with p0830, a destination vector used for Oryza sativa transformation. This vector contains as functional elements within the T-DNA borders: a plant selectable marker; a plant screenable marker; and a Gateway cassette intended for LR in vivo recombination with the sequence of interest already cloned in the entry clone. A prolamin promoter for endosperm-specific expression (PRO0090; SEQ ID NO: 3) is located upstream of this Gateway cassette.

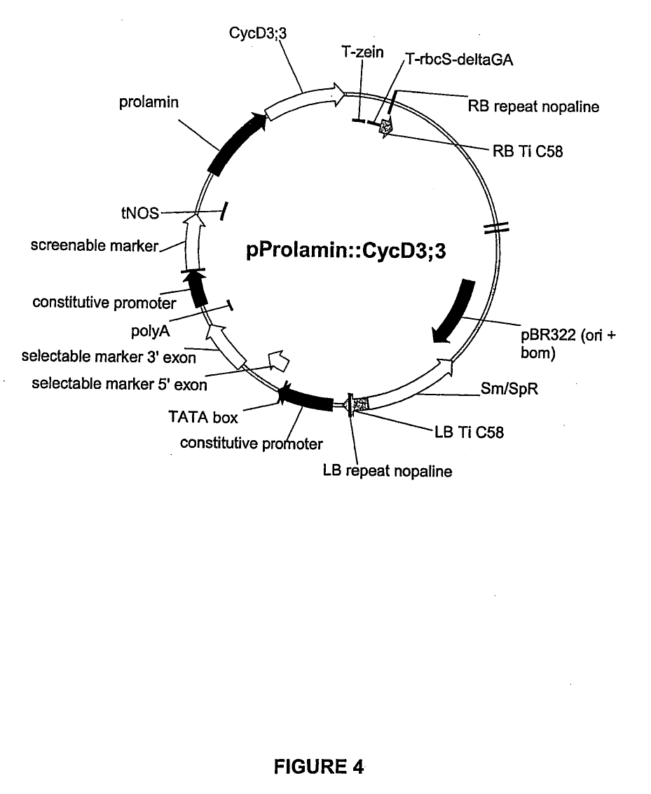

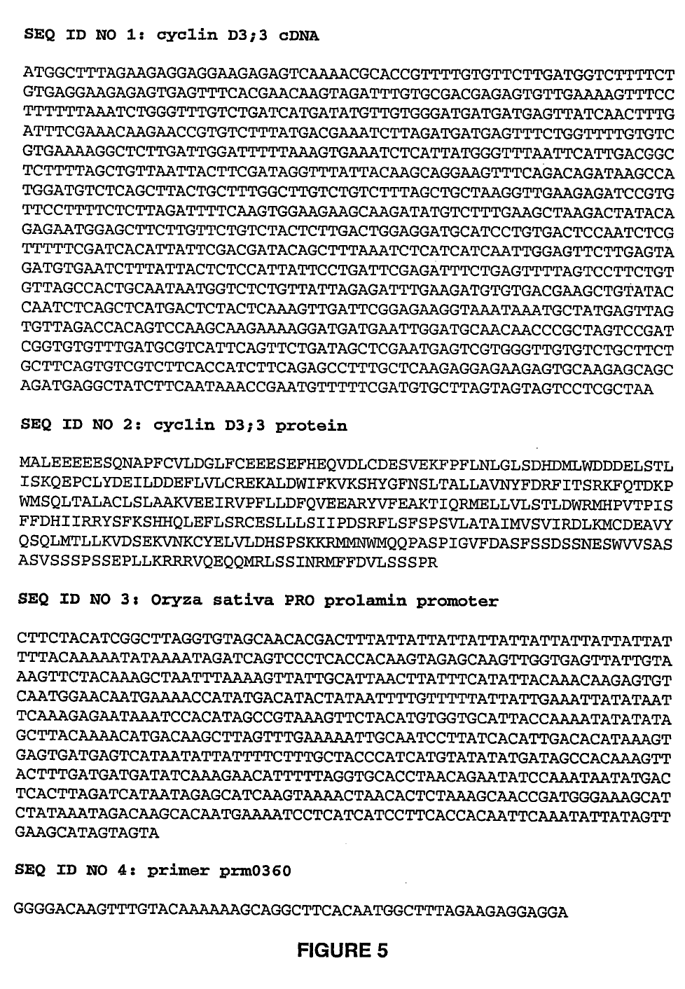

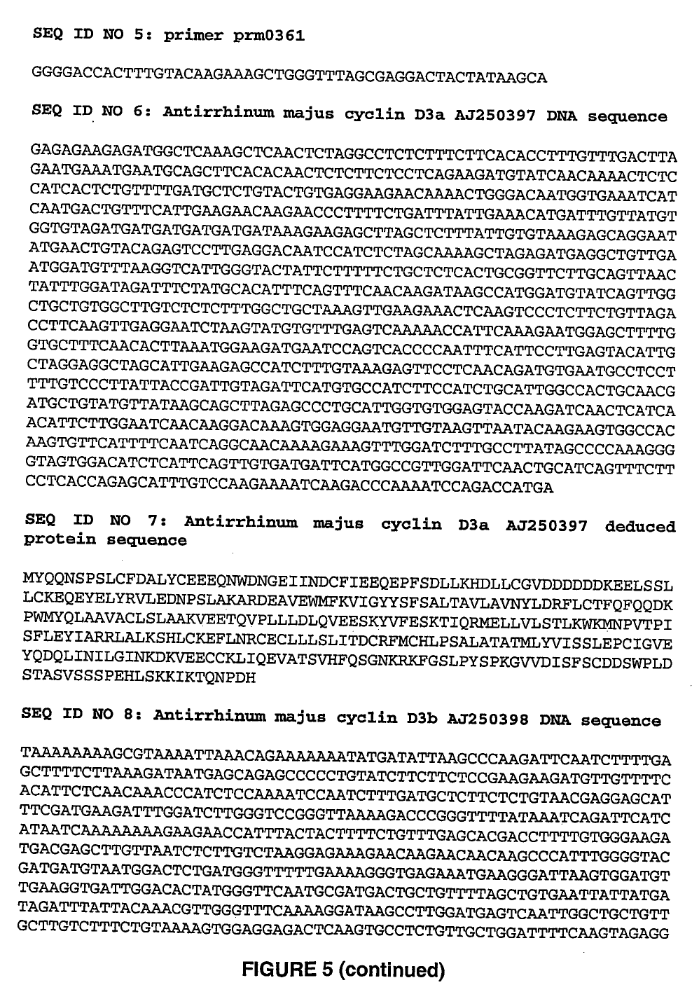

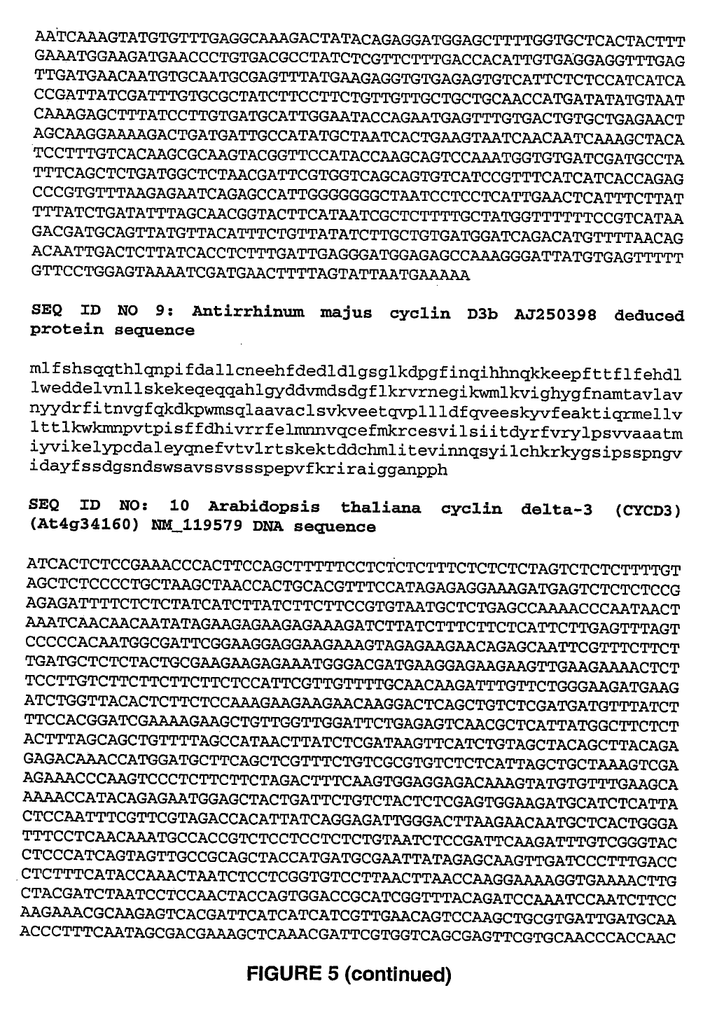

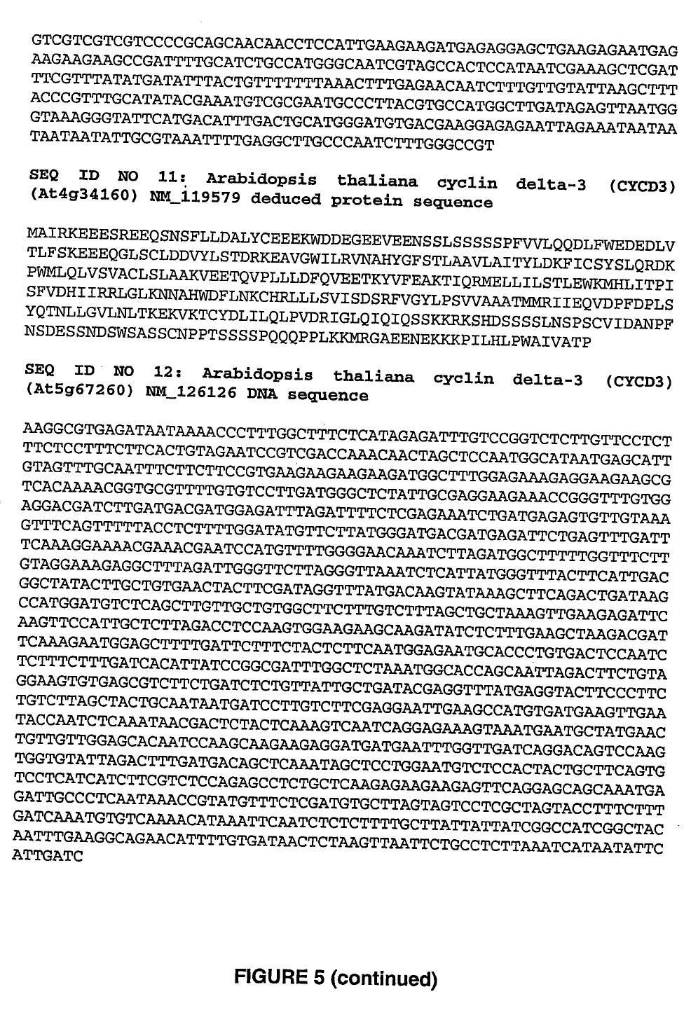

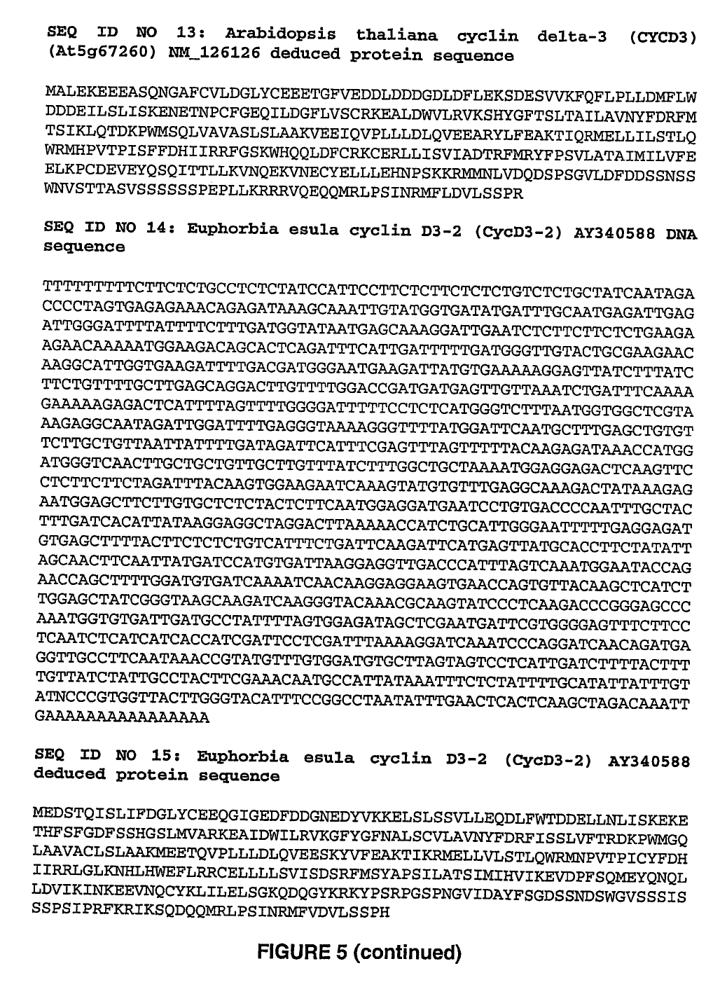

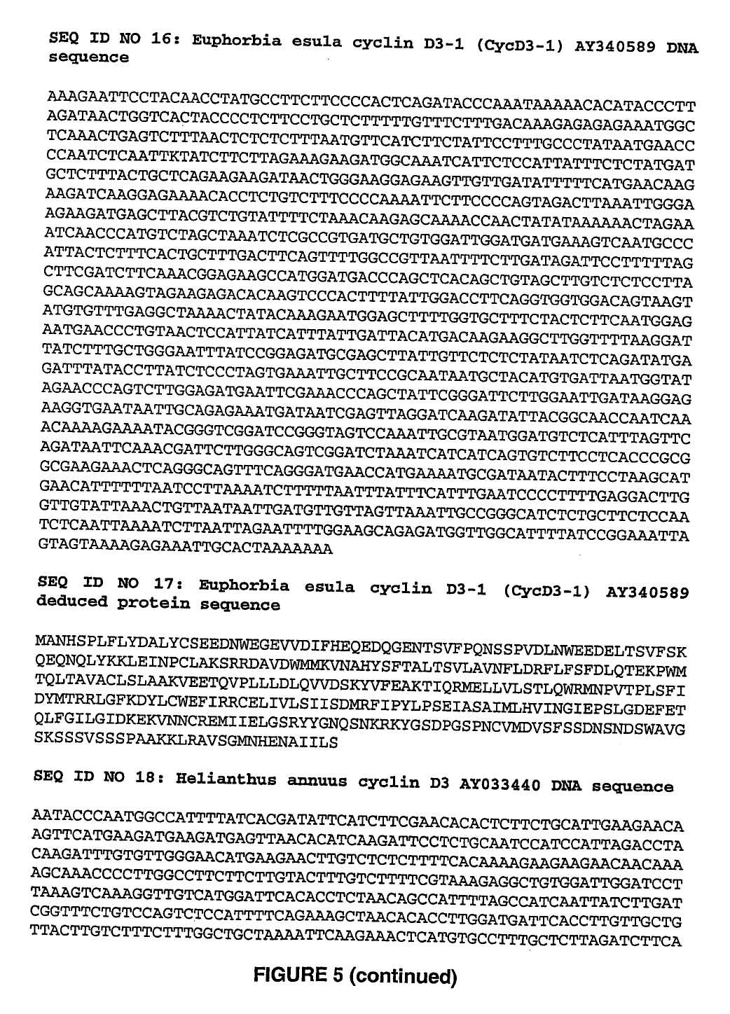

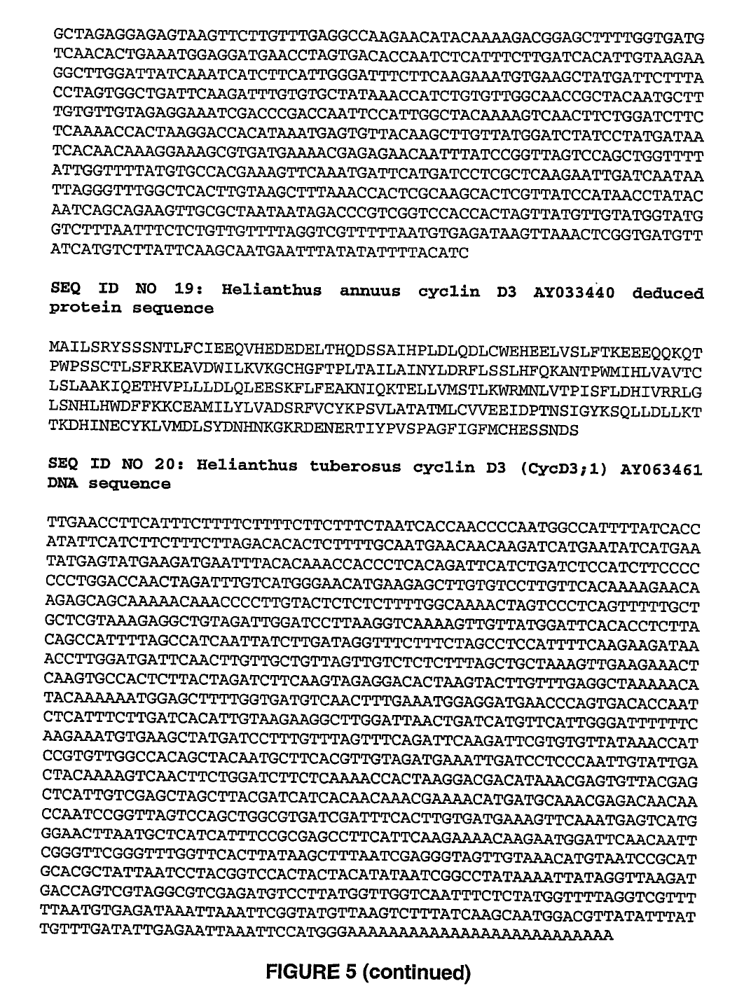

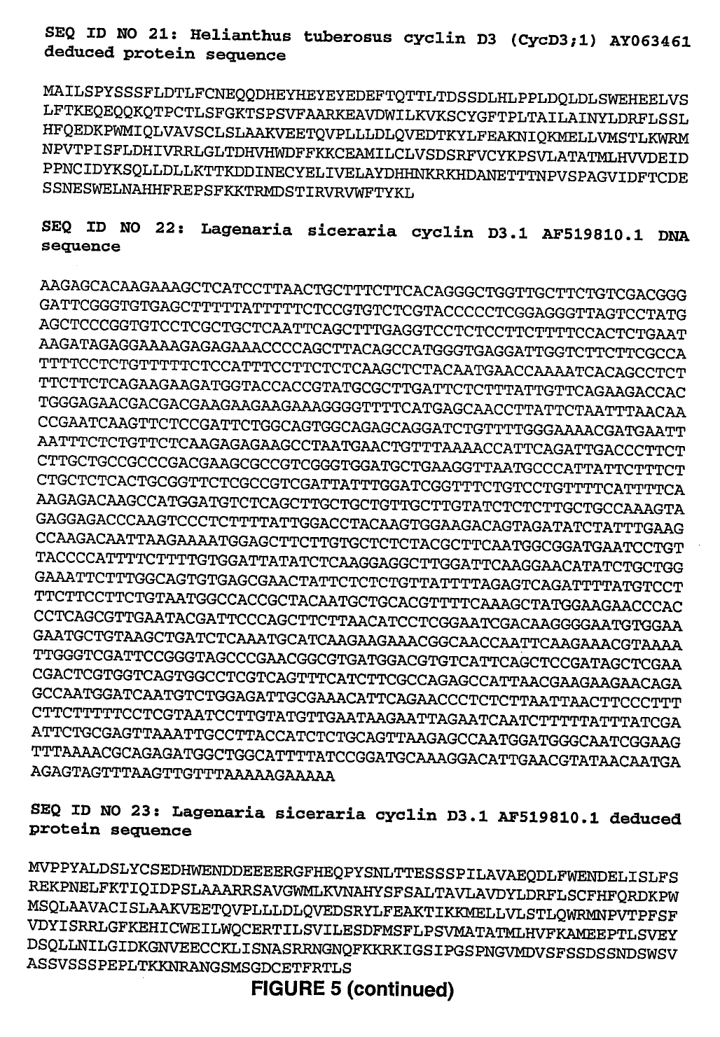

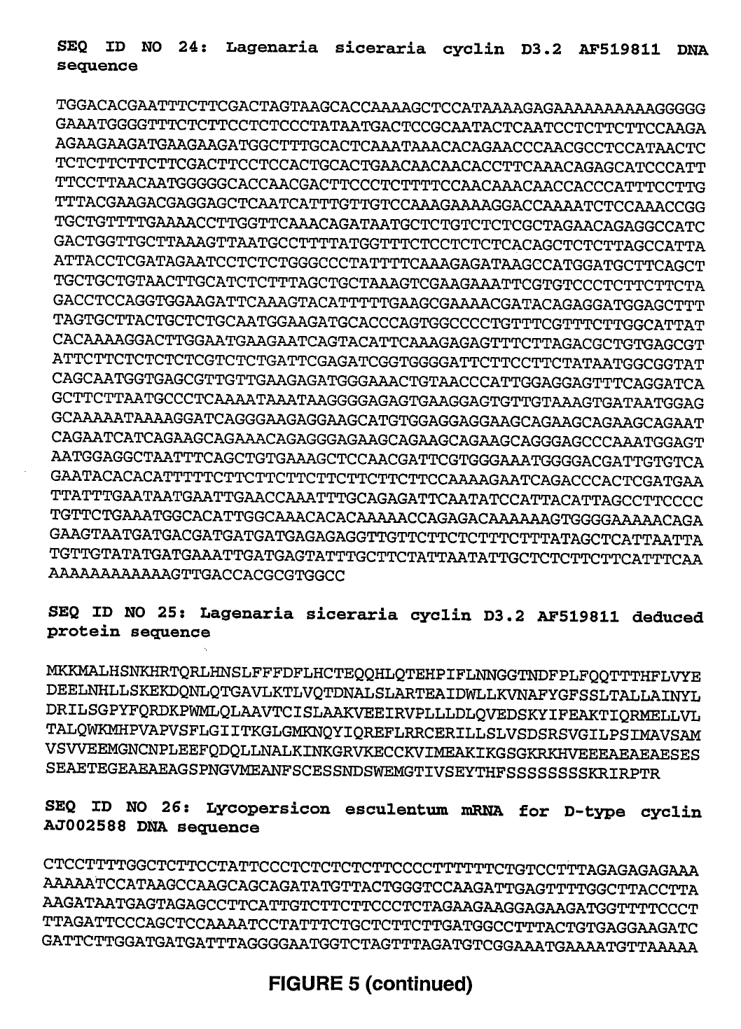

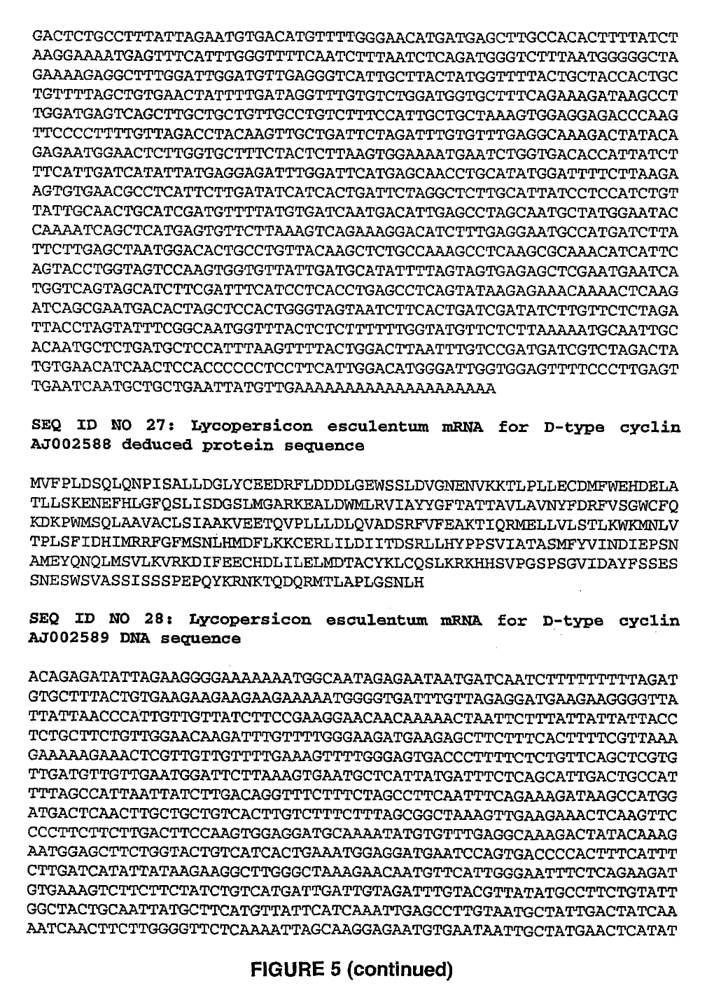

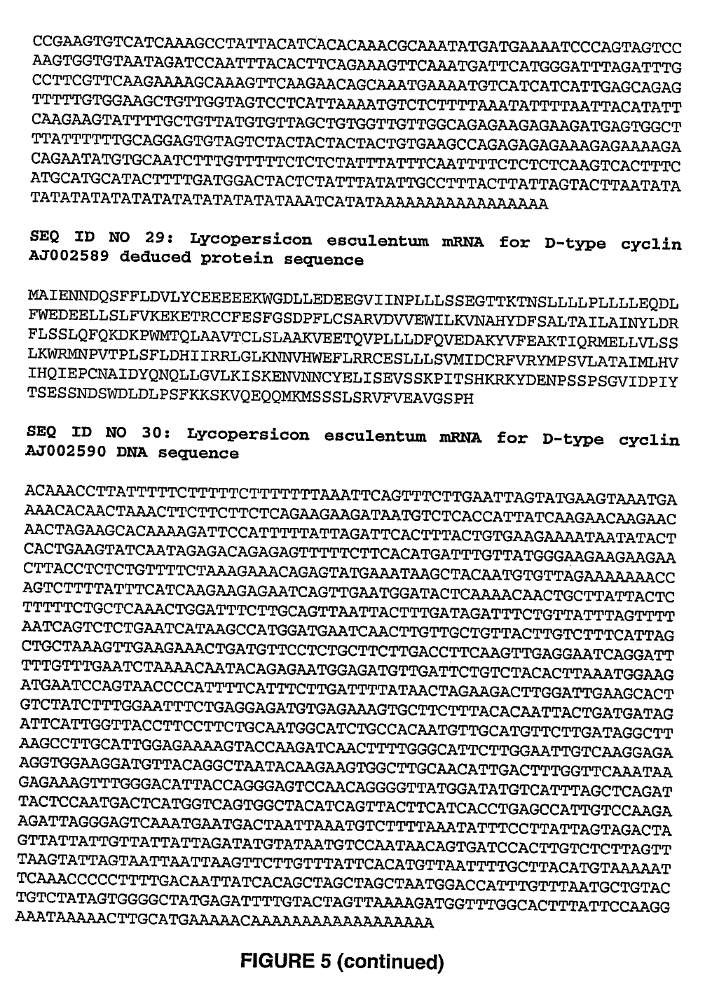

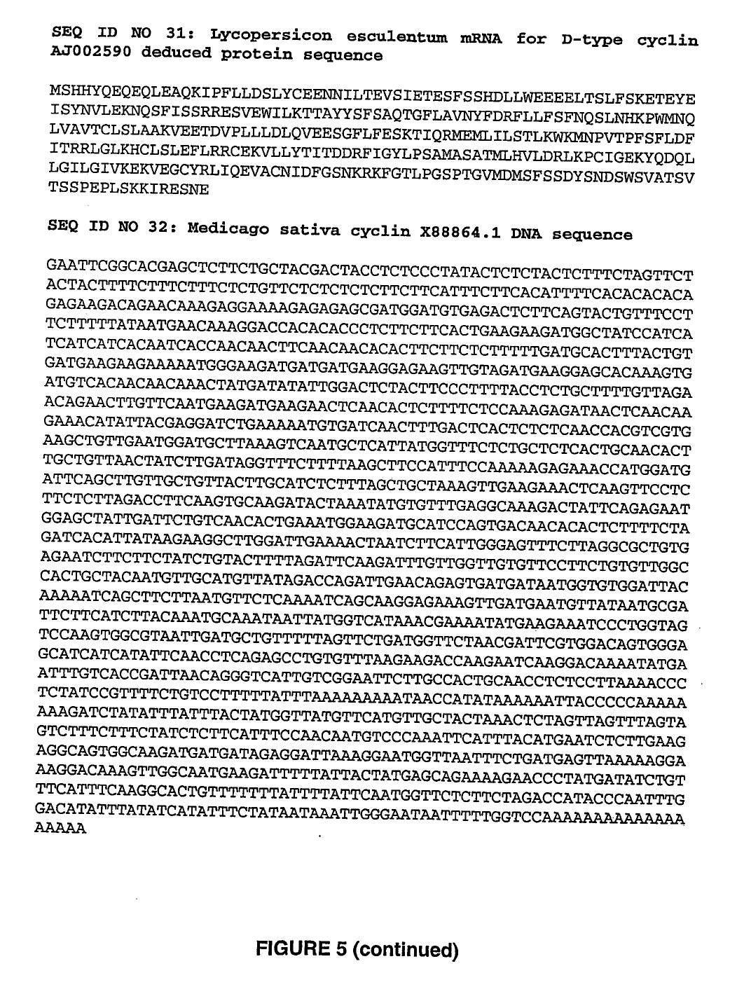

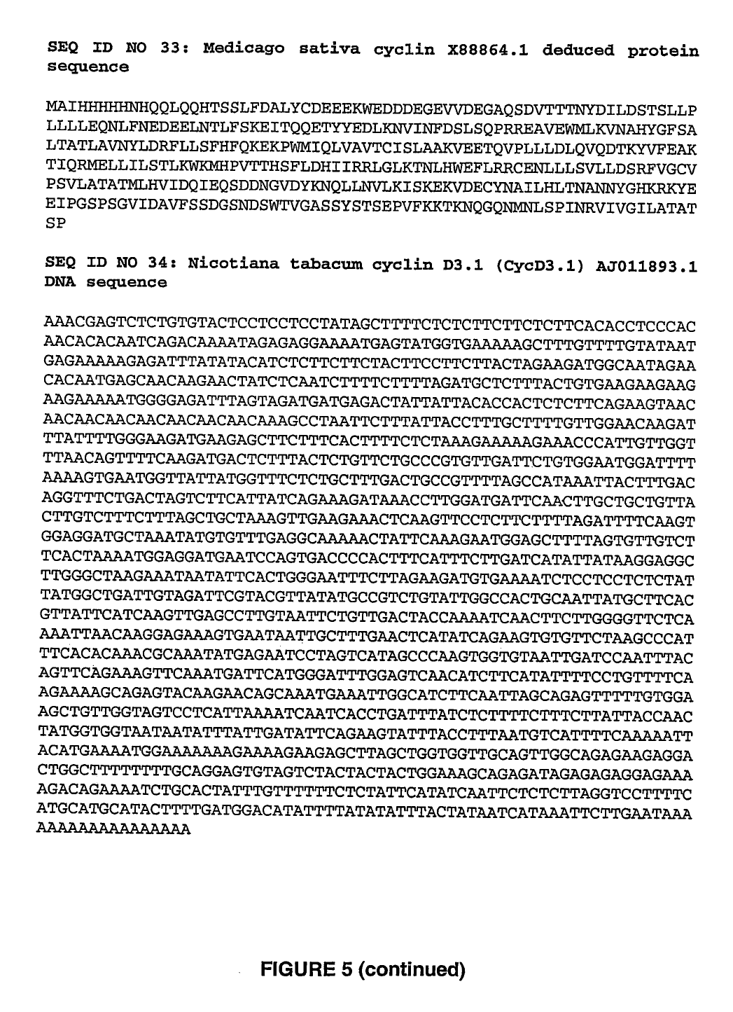

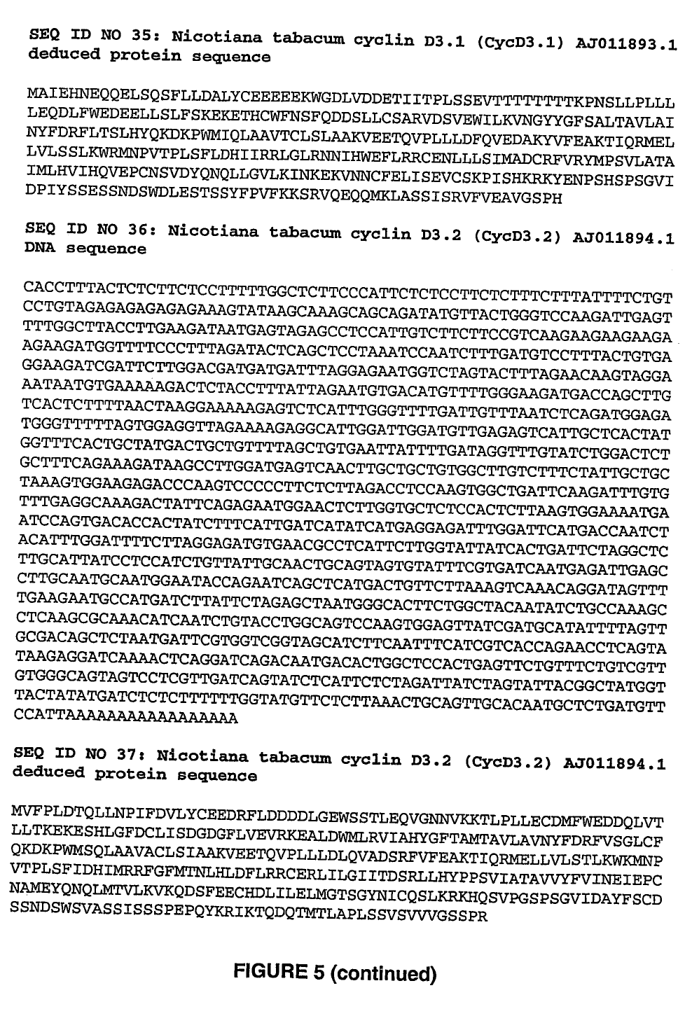

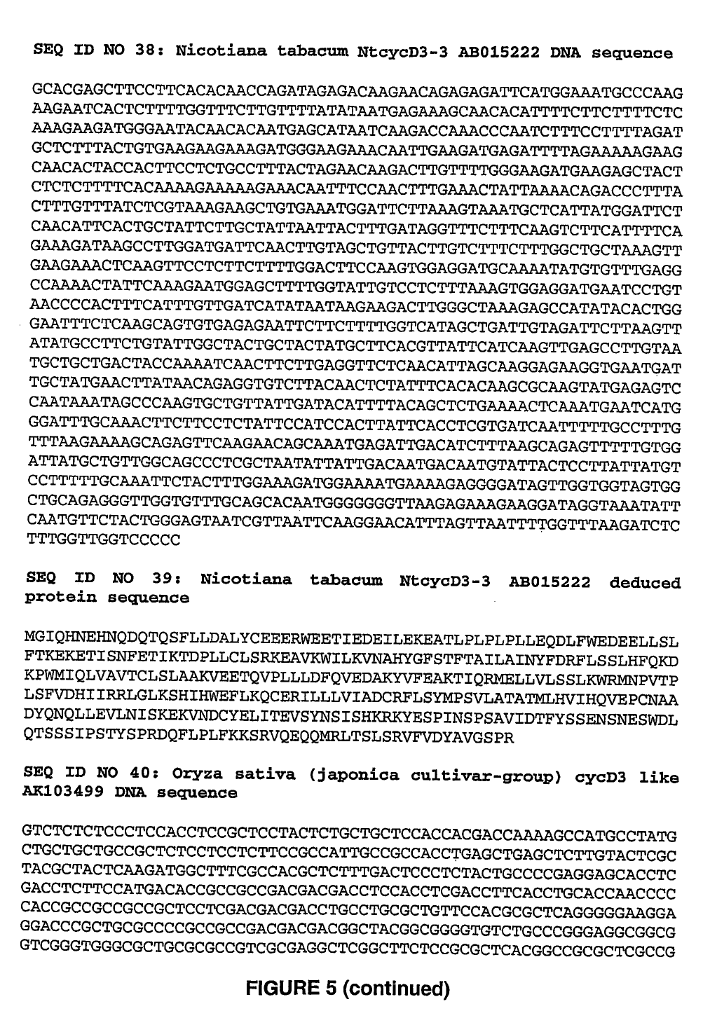

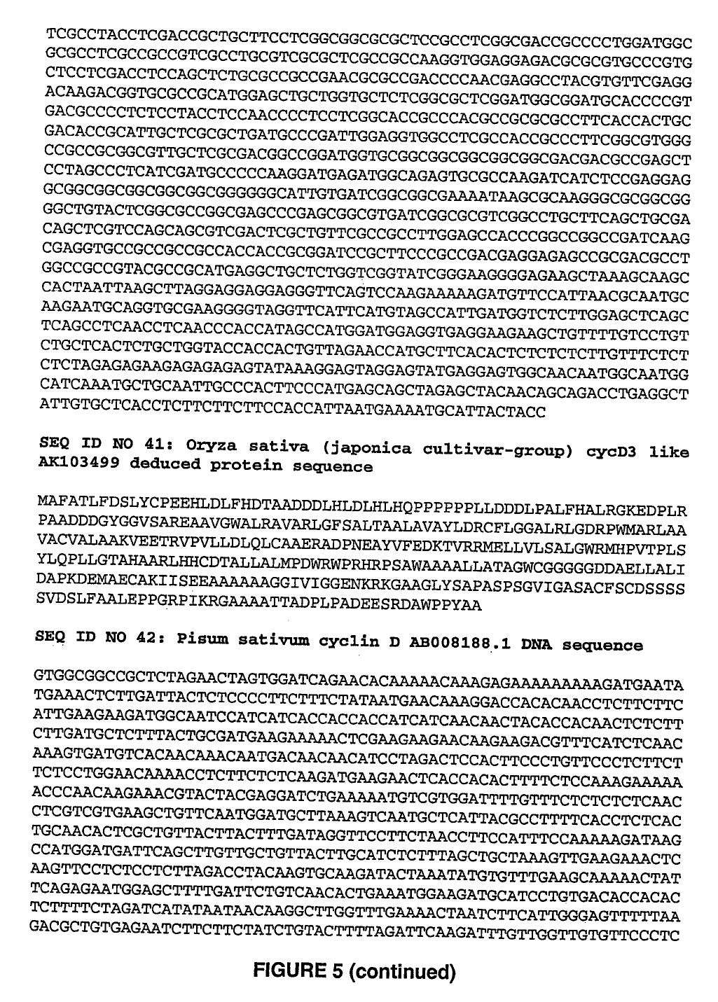

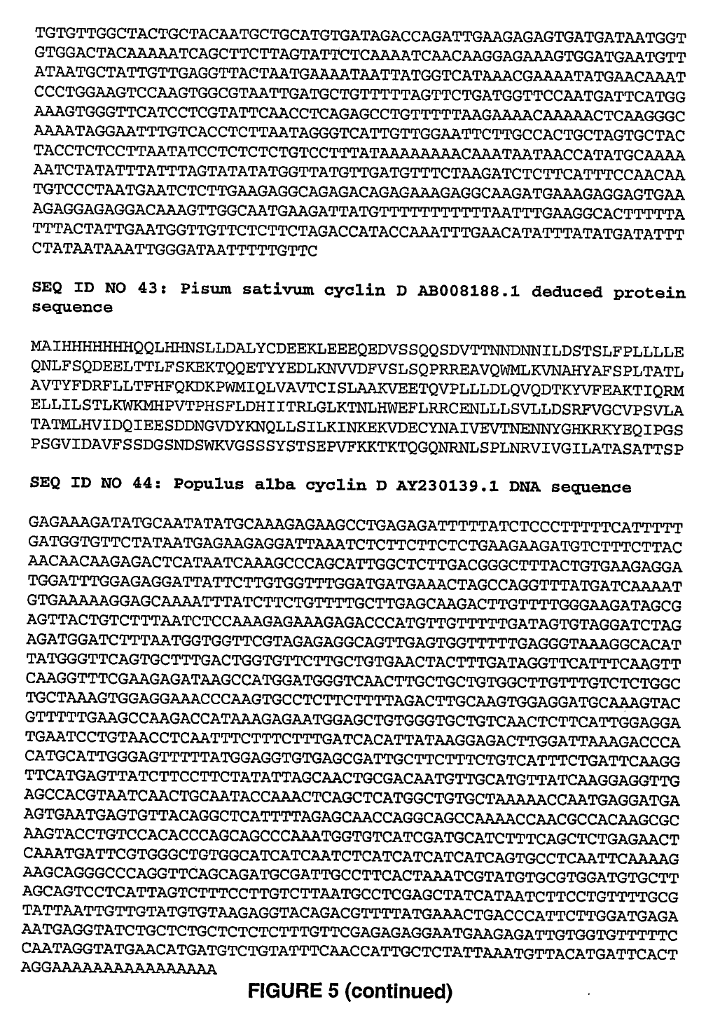

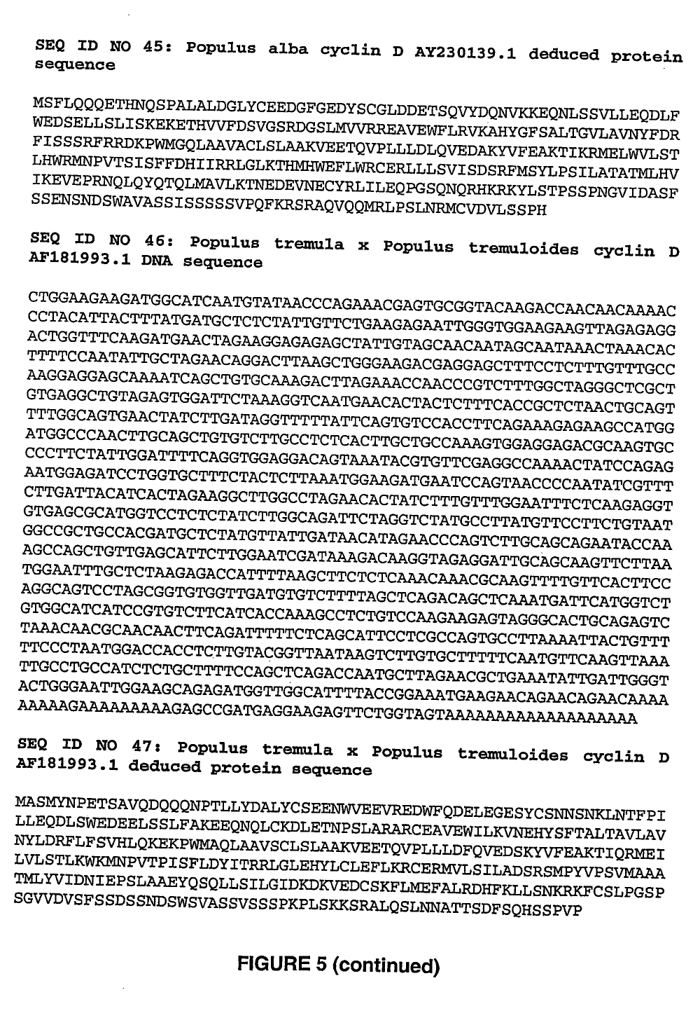

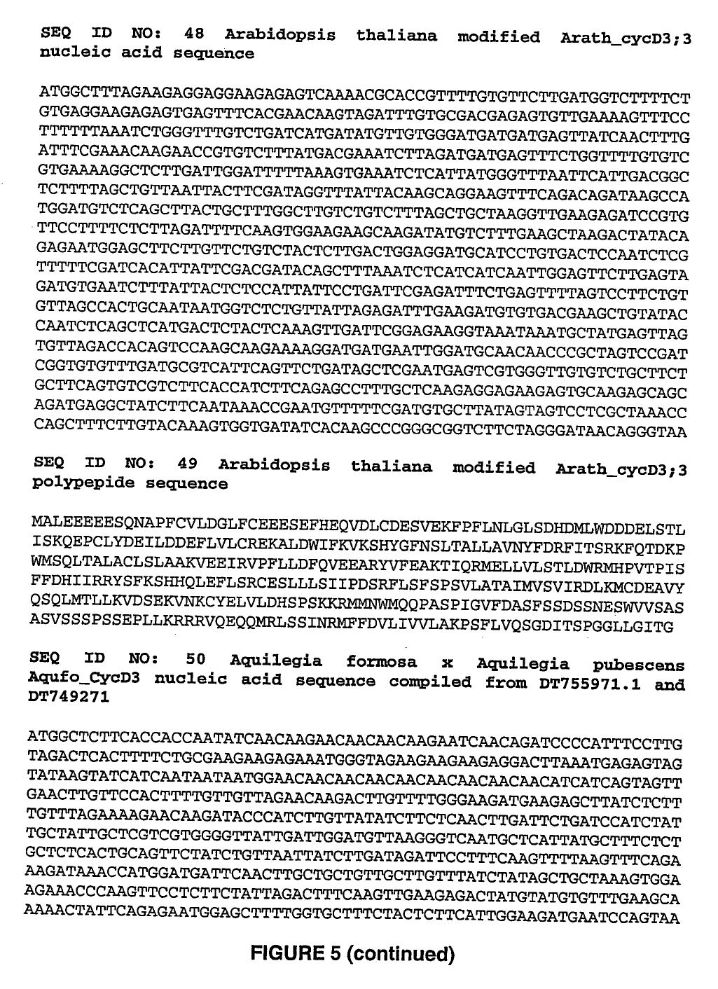

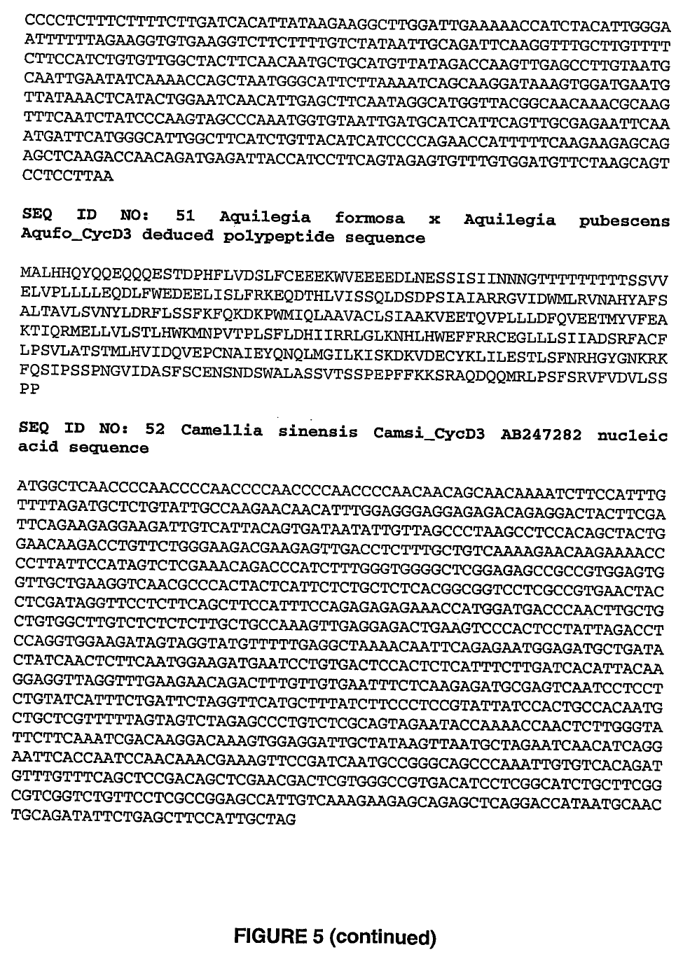

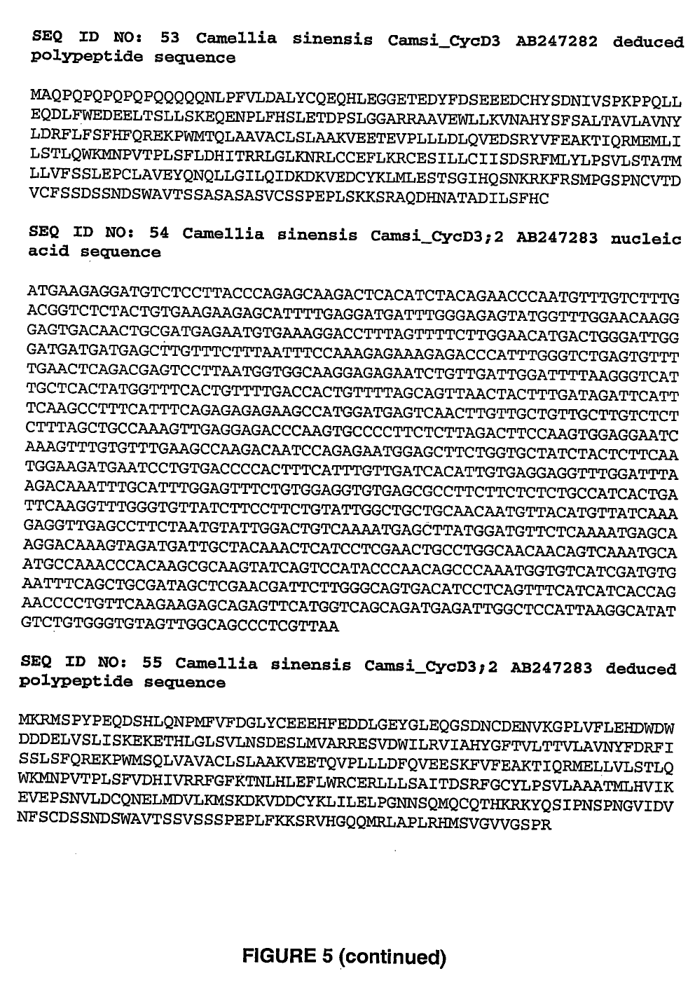

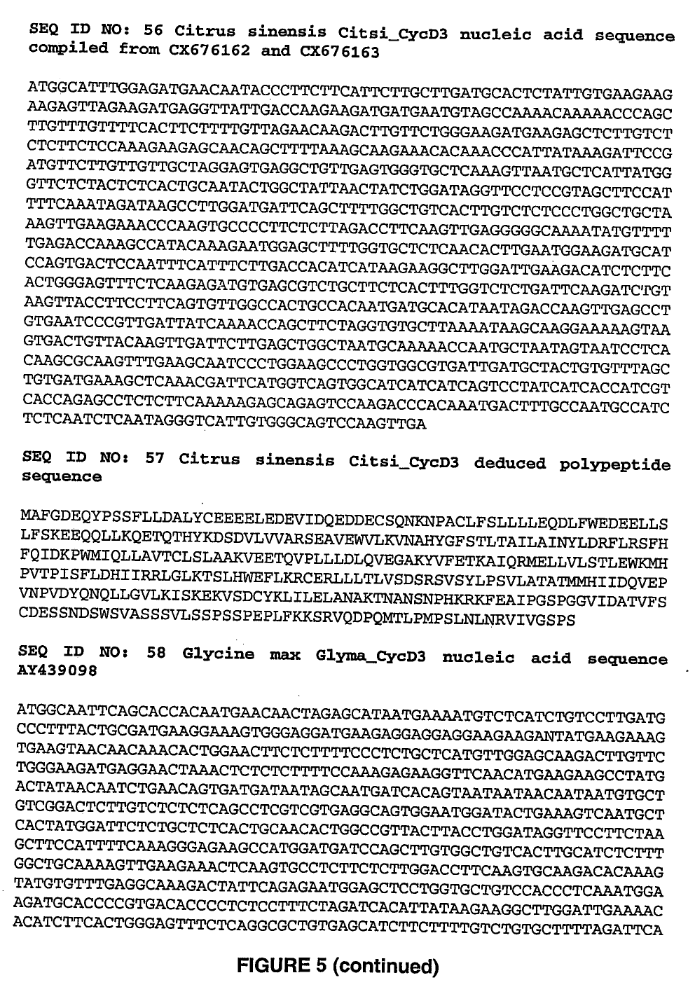

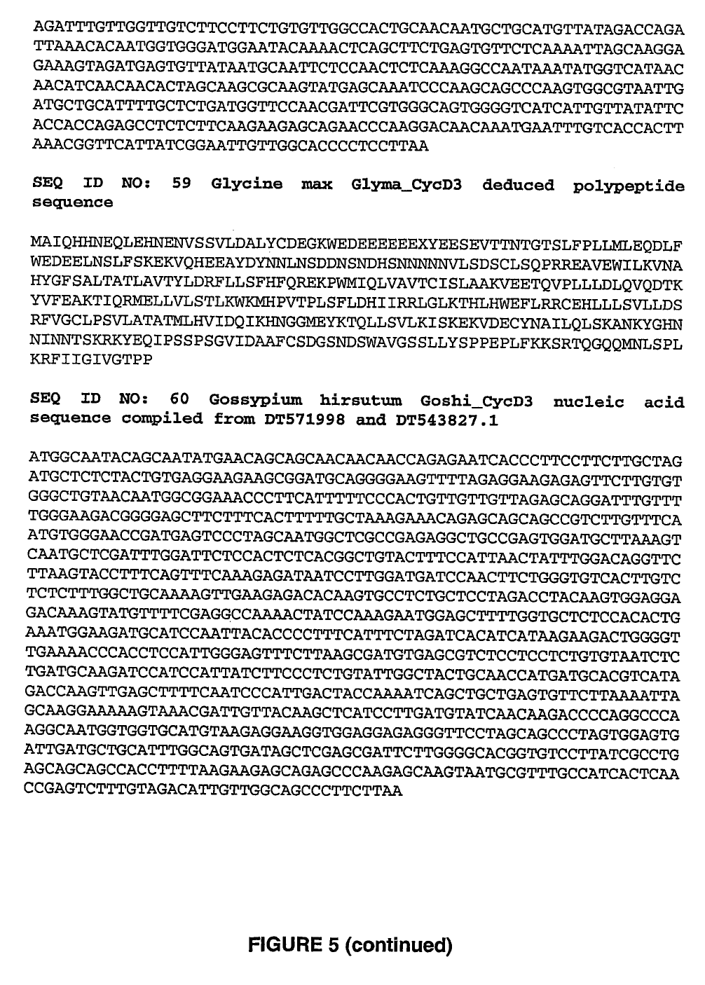

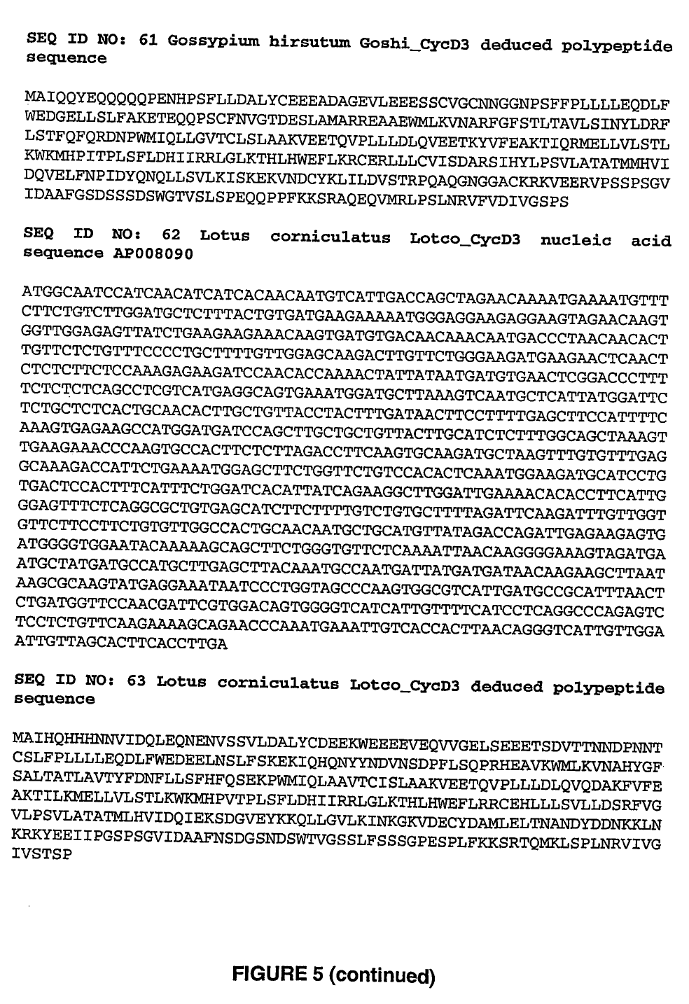

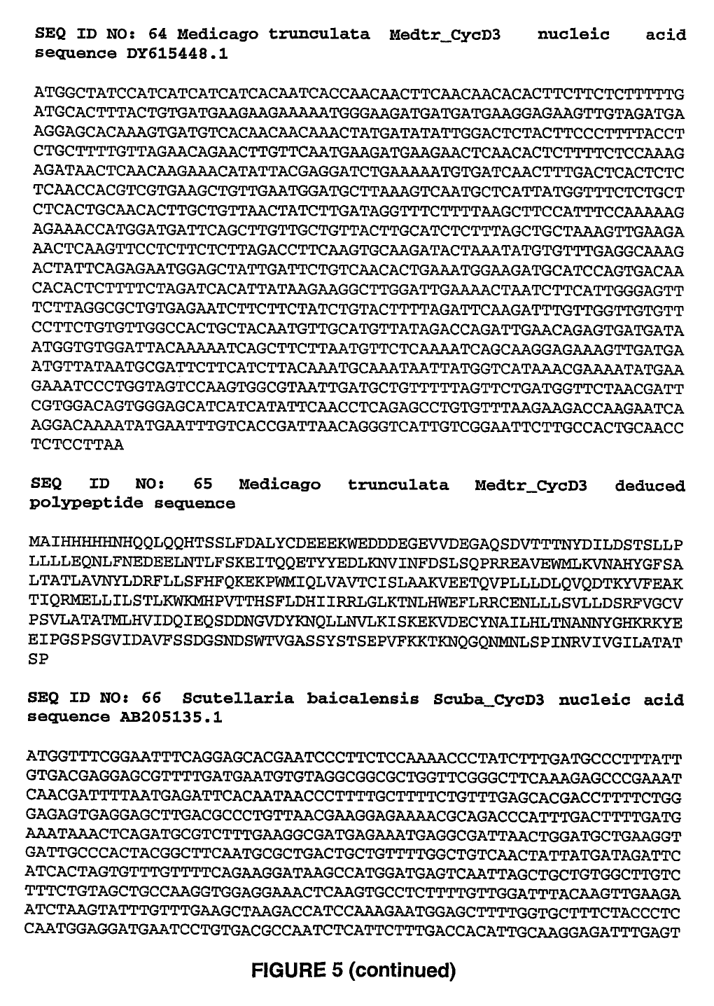

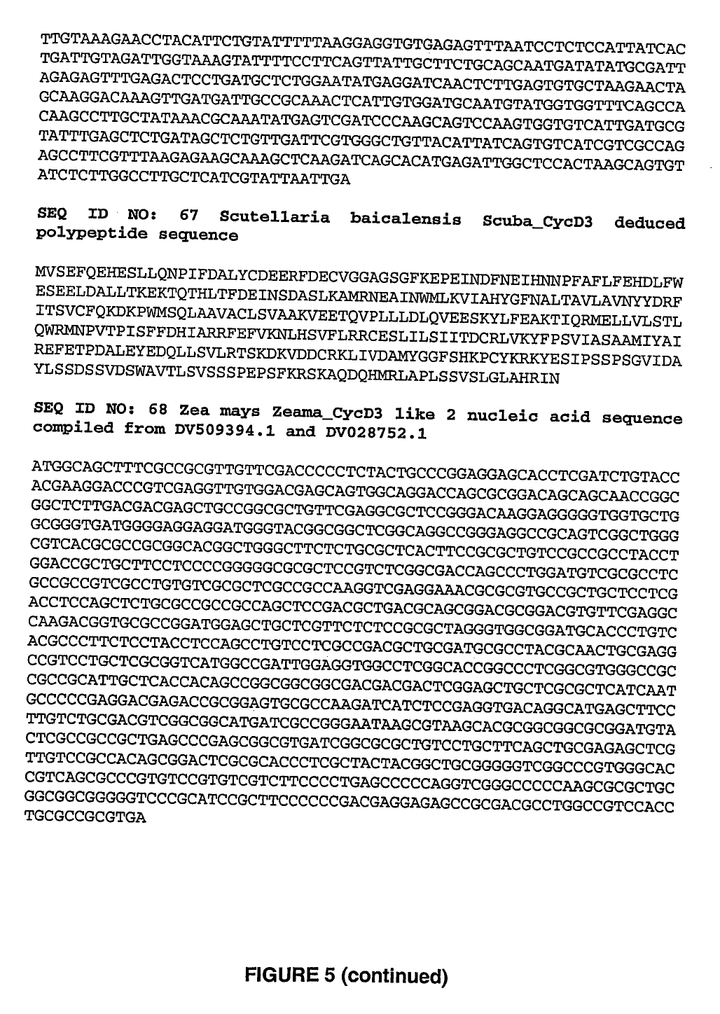

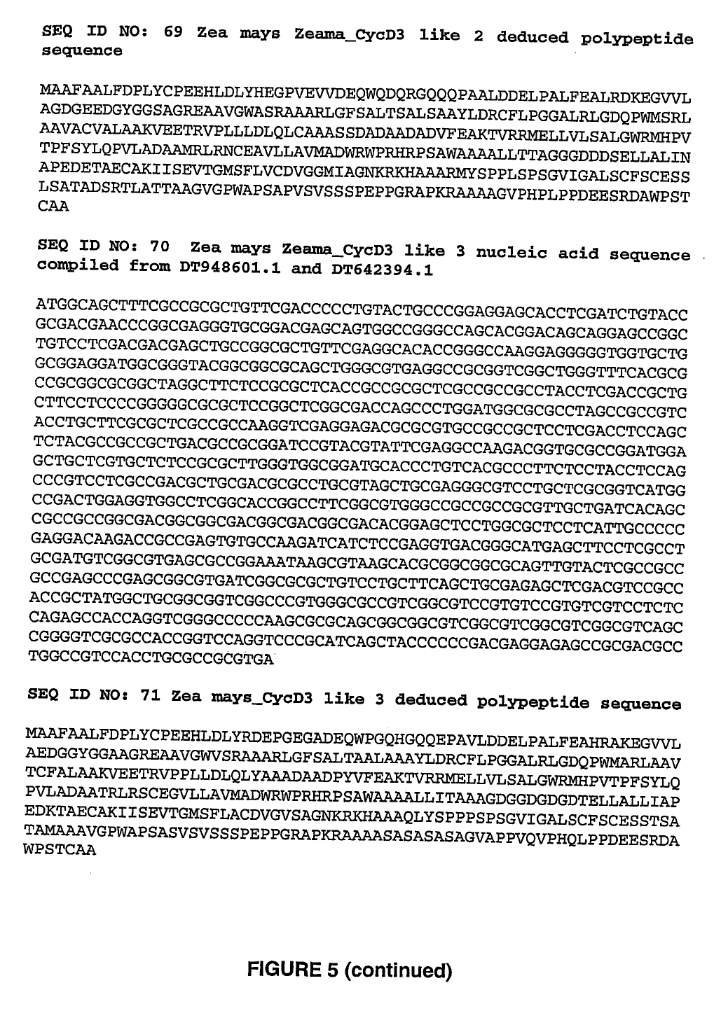

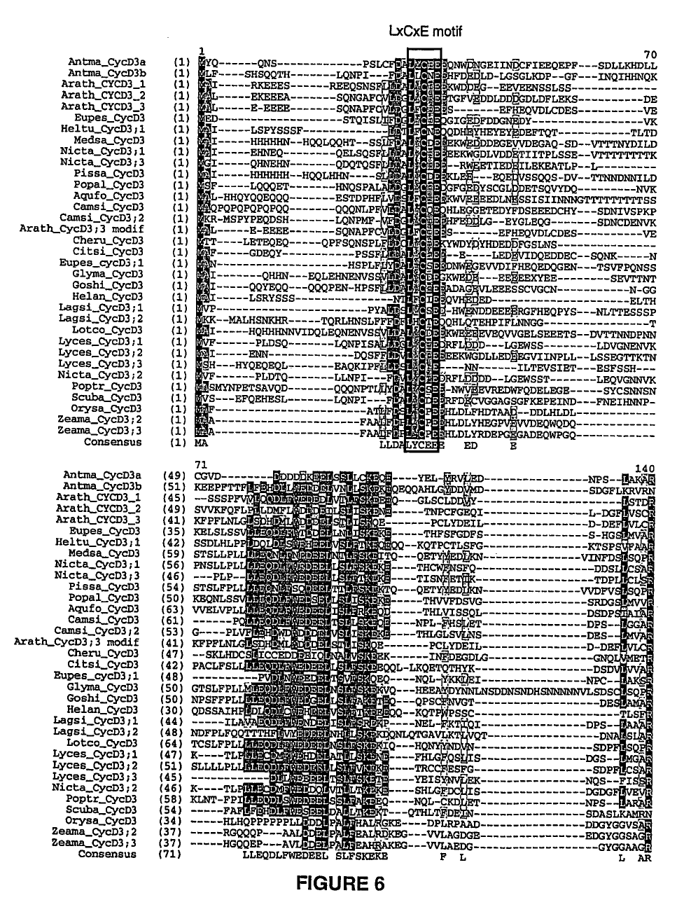

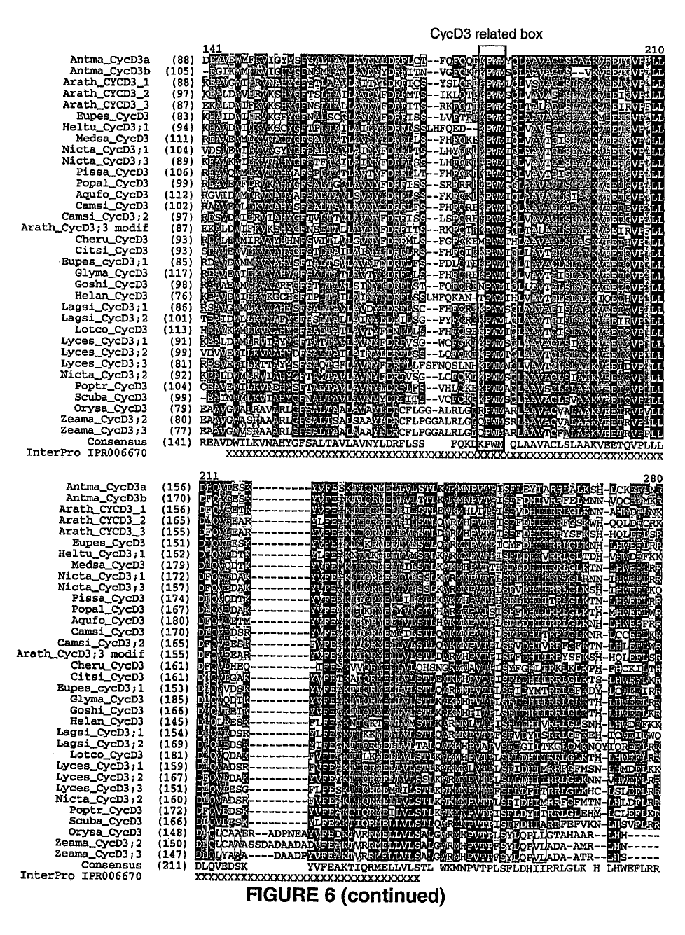

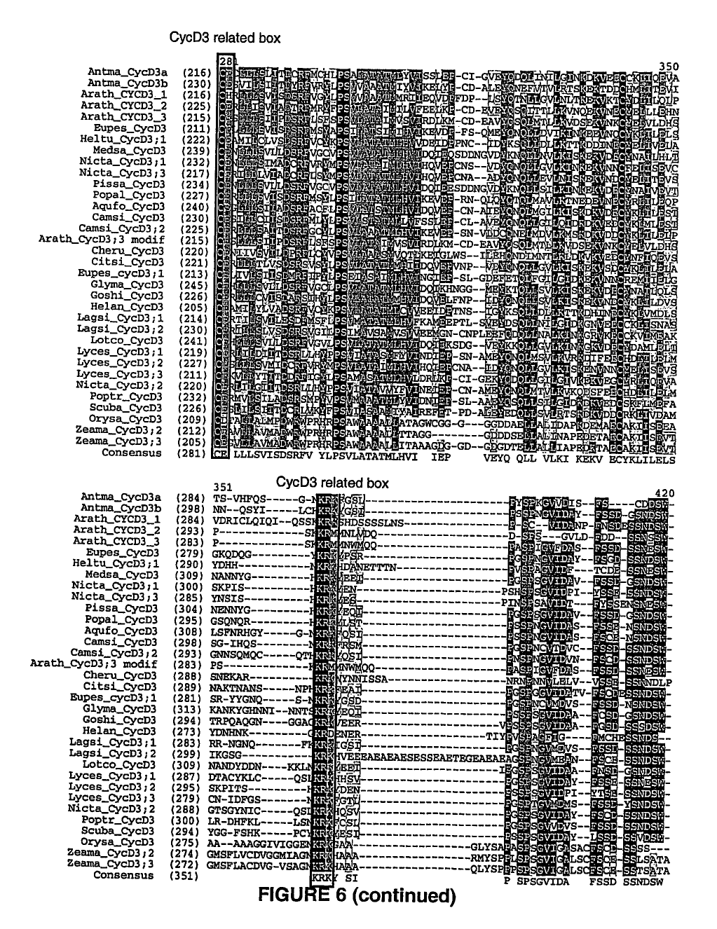

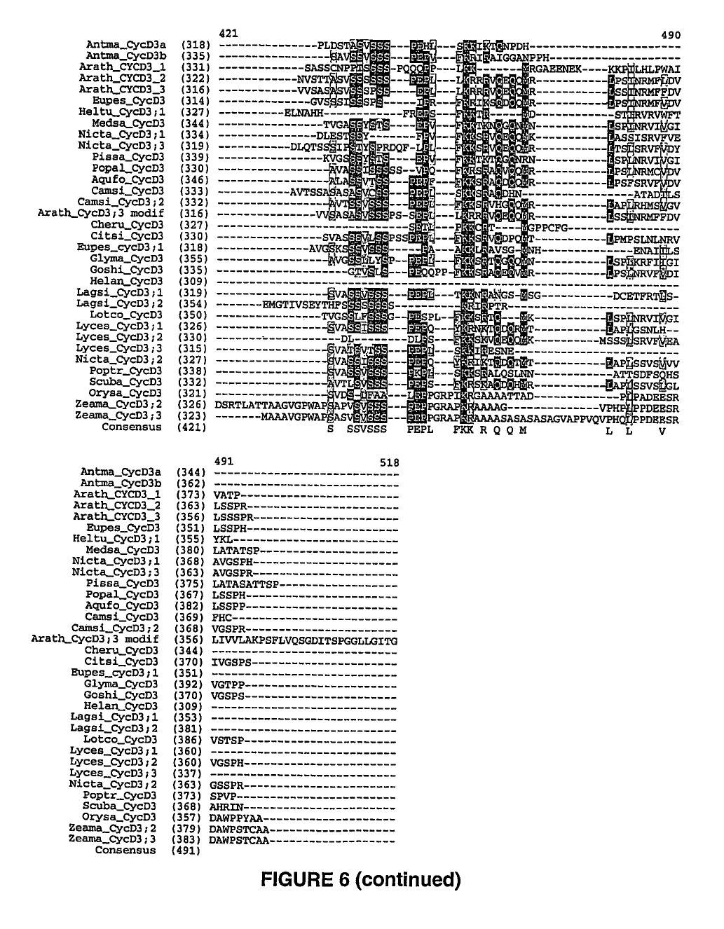

After the LR recombination step, the resulting expression vector (see FIG. 4) was transformed into Agrobacterium strain LBA4404 and subsequently to Oryza sativa plants. The resulting expression vector as shown in FIG. 4 was transformed into Agrobacterium and subsequently into Oryza sativa plants. Transformed rice plants were allowed to grow and were then examined for the parameters described in Example 3.

### Example 3

### Evaluation and Results

Approximately 15 to 20 independent T0 rice transformants were generated. The primary transformants were transferred from tissue culture chambers to a greenhouse for growing and harvest of T1 seed. Six events, of which the T1 progeny segregated 3:1 for presence/absence of the transgene, were retained. For each of these events, approximately 10 T1 seedlings containing the transgene (hetero- and homo-zygotes), and approximately 10 T1 seedlings lacking the transgene (nullizygotes), were selected by monitoring visual marker expression. The transgenic plants and the corresponding nullizygotes were grown side-by-side at random positions. From the stage of sowing until the stage of maturity the plants were passed several times through a digital imaging cabinet. At each time point digital images (2048×1536 pixels, 16 million colours) were taken of each plant from at least 6 different angles.

Five T1 events were further evaluated in the T2 generation following the same evaluation procedure as for the T1 generation but with more individuals per event.

### Statistical Analysis: T-Test and F-Test

A two factor ANOVA (analysis of variants) was used as a statistical model for the overall evaluation of plant phenotypic characteristics. An F-test was carried out on all the parameters measured of all the plants of all the events transformed with the gene of the present invention. The F-test was carried out to check for an effect of the gene over all the transformation events and to verify for an overall effect of the gene, also known as a global gene effect. The threshold for significance for a true global gene effect was set at a 5% probability level for the F-test. A significant F-test value points to a gene effect, meaning that it is not only the presence or position of the gene that is causing the differences in phenotype.

To check for an effect of the genes within an event, i.e., for a line-specific effect, a t-test was performed within each event using data sets from the transgenic plants and the corresponding null plants. “Null plants” or “null segregants” or “nullizygotes” are the plants treated in the same way as the transgenic plant, but from which the transgene has segregated. Null plants can also be described as the homozygous negative transformed plants. The threshold for significance for the t-test is set at 10% probability level. The results for some events can be above or below this threshold. This is based on the hypothesis that a gene might only have an effect in certain positions in the genome, and that the occurrence of this position-dependent effect is not uncommon. This kind of gene effect is also named herein a “line effect of the gene”. The p-value is obtained by comparing the t-value to the t-distribution or alternatively, by comparing the F-value to the F-distribution. The p-value then gives the probability that the null hypothesis (i.e., that there is no effect of the transgene) is correct.

### 3.1 Seed-Related Parameter Measurements

The mature primary panicles were harvested, bagged, barcode-labelled and then dried for three days in the oven at 37° C. The panicles were then threshed and all the seeds were collected and counted. The filled husks were separated from the empty ones using an air-blowing device. The empty husks were discarded and the remaining fraction was counted again. The filled husks were weighed on an analytical balance. This procedure resulted in the set of seed-related parameters described below.

### 3.1.1 Total Number of Flowers Per Panicle

The total number of flowers per panicle as defined in the present invention is the ratio between the total number of seeds and the number of mature primary panicles. The percentage difference between two significant transgenic events and their corresponding nullizygotes in T2 is shown in Table 5. The P value of the significant events in the T2 evaluation is also shown. A significant P value indicates that the presence of the transgene relates to the increase in total number of flowers per panicle.

TABLE 5
Total number of flowers per panicle

| | % increase in T2 | P value per event |
|---|---|---|
| Significant event 1 | 13 | 0.0286 |
| Significant event 2 | 22 | 0.0007 |

### 3.1.2 Total Seed Yield

The total seed yield was measured by weighing all filled husks harvested from a plant. The percentage difference between three significant transgenic events and their corresponding nullizygotes in T2 is shown in Table 6. The P value of the significant events in the T2 evaluation is also shown. A significant P value indicates that the presence of the transgene relates to the increase in total seed yield.

TABLE 6
Total seed yield

| | % increase in T2 | P value per event |
|---|---|---|
| Significant event 1 | 31 | 0.1306 |
| Significant event 2 | 36 | 0.0826 |
| Significant event 3 | 37 | 0.0005 |

### 3.1.3 TKW

TKW in the present invention is extrapolated from the number of filled seeds counted and their total weight. The percentage difference between three significant transgenic events and their corresponding nullizygotes in T2 is shown in Table 7. The P value of the significant events in the T2 evaluation is also shown. A significant P value indicates that the presence of the transgene relates to the increase in TKW.

TABLE 7
TKW

| | % increase in T2 | P value per event |
|---|---|---|
| Significant event 1 | 6 | 0.0006 |
| Significant event 2 | 5 | 0.0009 |
| Significant event 3 | 4 | 0.0165 |

### 3.1.4 Harvest Index of Plants

The harvest index in the present invention is defined as the ratio between the total seed yield and the above ground area (mm²), multiplied by a factor 10⁶. The percentage difference between the three significant transgenic events and their corresponding nullizygotes in T2 is shown in Table 8. The P value of the significant events in the T2 evaluation is also shown. A significant P value indicates that the presence of the transgene relates to the increase in harvest index.

TABLE 8
Harvest Index

| | % increase in T2 | P value per event |
|---|---|---|
| Significant event 1 | 30 | 0.0324 |
| Significant event 2 | 49 | 0.0014 |
| Significant event 3 | 15 | 0.0727 |

### Example 4

### Comparative Data pOleosin::Cyclin D3;3

Plants containing the above construct were produced and evaluated using the same procedures as described above for pProlamin::cyclinD3;3. The results of the T1 evaluation are shown in Tables 9 to 11 below. The percentage difference between transgenic plants and corresponding nullizygotes is shown in each of the tables. The p value of the F test is also shown.

TABLE 9
Aboveground Area
Aboveground area

| | % Difference | P value |
|---|---|---|
| T1 Overall | −12 | 0.0083 |

The p value of the F test was significant indicating that the expression of the transgene driven by this promoter significantly decreases aboveground area.

TABLE 10
Total Seed Weight
Total Seed Weight

| | % difference | P value |
|---|---|---|
| T1 Overall | −15 | 0.0858 |

The results show that the total weight of the seeds of transgenic plants was lower than the total seed weight of corresponding nullizygotes.

TABLE 11
Number of Filled Seeds
Number of Filled Seeds

| | % difference | P value |
|---|---|---|
| T1 Overall | −17 | 0.0572 |

The results show that the number of filled seeds of transgenic plants was lower than the number of filled seeds of corresponding nullizygotes.

1. A method for increasing plant yield relative to corresponding wild type plants, comprising increasing expression in a plant of a nucleic acid encoding a cyclin D3 (CYCD3) polypeptide under the control of a promoter capable of preferentially expressing the nucleic acid in the endosperm of seeds and optionally selecting for plants having increased yield.

2. The method according to claim 1, wherein said increased expression in the endosperm of seeds is effected by introducing a genetic modification in the locus of a gene encoding a CYCD3 polypeptide.

3. The method according to claim 2, wherein said genetic modification is effected by one of: T-DNA activation, TILLING, site-directed mutagenesis or directed evolution.

4. A method for the production of a transgenic plant having increased yield or for increasing plant yield relative to a corresponding wild type plant, comprising introducing and expressing in a plant or plant cell a nucleic acid encoding a CYCD3 polypeptide under the control of a promoter capable of preferentially expressing the nucleic acid in the endosperm of seeds or a construct comprising the nucleic acid; and cultivating the plant or plant cell under conditions promoting plant growth and development.

5. The method according to claim 4, wherein said nucleic acid encodes a portion of a CYCD3 polypeptide or is capable of hybridizing to a CYCD3-encoding nucleic acid.

6. The method according to claim 4, wherein said nucleic acid encodes an orthologue or paralogue of the CYCD3 protein of SEQ ID NO: 2.

7. The method according to claim 4, wherein said CYCD3-encoding nucleic acid is of plant origin.

8. The method according to claim 4, wherein said CYCD3-encoding nucleic acid is operably linked to an endosperm-specific promoter.

9. The method according to claim 8, wherein said endosperm-specific promoter is a prolamin promoter.

10. The method according to claim 1, wherein said increased yield is increased seed yield.

11. The method according to claim 1, wherein said increased yield is selected from: increased number of flowers per panicle, increased total seed yield, increased TKW and increased harvest index.

12. A plant obtained by the method according to claim 1.

13. A construct comprising:
(i) a nucleic acid encoding a CYCD3 polypeptide;
(ii) one or more control sequences capable of preferentially driving expression of the nucleic acid sequence of (i) in the endosperm of seeds; and optionally (iii) a transcription termination sequence.

14. The construct according to claim 13, wherein said control sequence is an endosperm-specific promoter.

15. The construct according to claim 14, wherein said endosperm-specific promoter is a prolamin promoter.

16. The construct according to claim 15, wherein said prolamin promoter is as represented by SEQ ID NO: 3.

17. A plant transformed with the construct according to claim 13.

18. (canceled)

19. A transgenic plant having increased yield resulting from a nucleic acid encoding a CYCD3 polypeptide introduced and expressed into said plant, under the control of a promoter capable of preferentially expressing the nucleic acid in the endosperm of seeds.

20. The transgenic plant according to claim 19, wherein said plant is a monocotyledonous plant.

21. Harvestable parts of the plant according to claim 19.

22. Harvestable parts of a plant according to claim 21 wherein said harvestable parts are seeds.

23. Products directly derived from a plant according to claim 19 and/or from harvestable parts of the plant.

24. (canceled)

25. The method according to claim 4, wherein said yield is increased seed yield selected from: increased number of flowers per panicle, increased total seed yield, increased TKW and increased harvest index.

26. The transgenic plant of claim 12, wherein said plant is a monocotyledonous plant.

27. The method of claim 7, wherein said CYCD3-encoding nucleic acid is from a dicotyledonous plant.
